(19) United States
(12) Patent Application Publication
(10) Pub. No.: US 2022/0106598 A1
(43) Pub. Date: Apr. 7, 2022

(54) HOMOLOGOUS RECOMBINATION DIRECTED GENOME EDITING IN EUKARYOTES

(71) Applicant: PILLARGO, INC., San Diego, CA (US)

(72) Inventors: Ying Wu, San Diego, CA (US); Jiagang Zhao, San Diego, CA (US)

(21) Appl. No.: 17/557,346

(22) Filed: Dec. 21, 2021

Related U.S. Application Data
(63) Continuation of application No. PCT/US2020/040596, filed on Jul. 2, 2020.
Application No. PCT/US2020/040596 is a continuation-in-part of application No. 16/556,054, filed on Aug. 29, 2019, now Pat. No. 10,851,370.
(60) Provisional application No. 62/871,495, filed on Jul. 8, 2019.
(60) Provisional application No. 62/965,661, filed on Jan. 24, 2020.
(60) Provisional application No. 62/871,495, filed on Jul. 8, 2019.

(51) Int. Cl. C12N 15/113; C12N 15/86; C12N 5/071

(52) U.S. Cl. CPC C12N 15/113; C12N 2310/14; C12N 5/0602; C12N 15/86

(57) ABSTRACT

Disclosed herein are synthetic nucleic acids comprising a nucleic acid sequence that encodes an ANAGO that is a species-specific to a eukaryote, such as a human, and compositions comprising ANAGO, such as a human ANAGO, and donor molecules for use in homologous recombination directed targeted gene editing in the eukaryote, such as in human cells.

### CROSS-REFERENCE TO RELATED APPLICATION

This application is a continuation of International Pat. App. No. PCT/US2020/040596, filed Jul. 2, 2020; which claims benefit of U.S. Provisional App. Nos. 62/871,495, filed Jul. 8, 2019, and 62/965,661, filed Jan. 24, 2020; International Pat. App. No. PCT/US2020/040596 is also a continuation-in-part of U.S. patent application Ser. No. 16/556,054, filed Aug. 29, 2019, now U.S. Pat. No. 10,851,370; which claims benefit of U.S. Provisional App. No. 62/871,495, filed Jul. 8, 2019; all of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Jul. 1, 2020, is named P3651_10001WO01_SL.txt and is 25,238 bytes in size.

U.S. patent application Ser. No. 16/556,054, filed on Aug. 29, 2019 is incorporated by reference herein for all sequence information contained therein.

### FIELD

The present disclosure relates to compositions comprising synthetic nucleic acids comprising human preferred codons that facilitate genome editing. The present invention also relates to methods of using the compositions comprising synthetic nucleic acids and donor nucleic acids for genetic engineering in human beings and other mammals, such as methods of precise genome editing in human cells including pluripotent stem cells.

### BACKGROUND

Genome editing has offered a powerful tool and unprecedented opportunity to study gene functions and to fight diseases by introducing a targeted genomic sequence change at a specific locus of a living cell or organism. Zinc finger nucleases (ZFNs), transcription activator-like effector nucleases (TALENs), and clustered regularly interspaced short palindromic repeats (CRISPR)-associated (Cas) nucleases are the ones that have been used successfully and efficiently by many laboratories. More recently, the RNA-guided endonucleases such as Cas9 and Cpf1 have gained more traction because of their relatively ease of manipulation. The user-friendly CRISPR-Cas9 is very efficient in making mutations via nonhomologous end joining (NHEJ) in human cancer cell lines such as 293T cells. It also can mediate homologous recombination (HR), but at a much lower efficiency in 293T cells and even lower in other biologically relevant primary cells such as human induced pluripotent stem cells (iPSCs).

The success of derivation of human embryonic stem cell (ESC) was reported in 1998, and subsequently human induced pluripotent stem cells (iPSCs), which can be directly converted from a somatic cell and is equivalent to ESC, were also created. These pluripotent stem cells (PSCs) can be utilized for disease modeling, ex vivo developmental, and mechanistic studies. More importantly, they can be used for regenerative medicine. Unfortunately, classical way of gene targeting based on homologous recombination has been proved remarkably inefficient in PSCs.

Due to the emergence of new genome editing technologies, such as ZFN, TALENs, and CRISPR, our capacity to genetically manipulate PSCs has been increased. Such manipulations include the creation of partial or full loss of function of gene mutations, the knock-in of reporter genes for tracing cell lineage during differentiation, and/or the creation of conditional knock-out to assess a gene function spatially (in a specific group of cells, an organ) or temporally (at a particular developmental stage or timepoint during adulthood). Nevertheless, precise gene editing remains challenging due to the low efficiency of homology directed repair (HDR) in PSCs.

Recently, it has been reported that it is feasible to achieve genome editing by using Natronobacterium gregoryi Argonaute (NgAgo) with a guide DNA oligo in human cells (Gao F, et al., (2016) Nat Biotechnol. 34(7):768-73). However, multiple labs have failed to reproduce this phenomenon claimed by Gao et al. thus far, which led to the withdrawal of this publication. Interestingly, an eye defect was observed by using an NgAgo approach in zebrafish. The phenotype was most likely caused by an NgAgo mediated gene knockdown effect, and no genetic modification was observed at the DNA level.

Argonautes use an orthogonal mechanism of immune surveillance and promise an entirely novel process for gene editing. Argonautes are a family of endonucleases that use 5′ phosphorylated short single-stranded nucleic acids as guides to cleave targets. Similar to Cas9 and Cpf1, Argonautes play key roles in gene expression repression and host defense against foreign nucleic acids. While Cas9 and Cpf1 are only naturally found in prokaryotes, members of Argonaute superfamily are reported to be present in many species (from bacteria to mammals). Although most Argonautes associate with single-stranded (ss) RNAs and play a central role in RNA silencing, some Argonautes bind ssDNAs and cleave target DNAs. It appears that DNA-guided Argonaute binding does not have a specific requirement for sequence or secondary structure. Argonautes are conserved throughout the bacterial and archaeal domains of living organisms. Their major functions are likely to be involved in DNA-guided DNA-interfering host defense systems.

An ideal gene editing tool should be able to introduce a desired base change, deletion or insertion into the genome in a precise, non-bias and error-free manner. Although CRISPR/Cas systems, originally derived from a bacterial host defense mechanism, are versatile and are currently widely used, they are error-prone and sequence-biased.

### SUMMARY

Clinical applications of genome editing demand a precise and/or desirably error-free DNA sequence alterations in human cells and tissues. This disclosure demonstrates that the desired base changes can be precisely incorporated into the stem cell genomic DNA by employing ANAGO Induced Sequence Editing (AISE) technology.

ANAGOs are engineered nucleases that are derived from the sequences of a protein superfamily Argonautes. In microbial organisms, Argonautes participate in host defense mechanisms against foreign genetic material invasion. This disclosure demonstrates that ANAGOs can mediate a precise gene editing event via DNA homologous recombination in human cells including induced pluripotent stem cells (iPSCs).

Disclosed herein are several novel codon-adapted Argonaute protein variants named ANAGO that are derived from microbial Argonaute proteins capable of editing a target nucleic acid sequence within a prokaryotic cell. The DNA coding sequences of these microbial Argonaute proteins, such as NgAgo, PfAgo, TtAgo and MjAgo, can be reengineered and adapted with the codons that have preferential usage in eukaryotes, such as humans to generate new synthetic DNA sequences that encodes the ANAGO that are species-specific to the eukaryotes, wherein the ANAGO conserves or retains the endonuclease activities, capable of editing a target nucleic acid sequence within a eukaryotic cell. Also disclosed herein are the uses of these ANAGO and one or more homologous donor nucleic acids to mediate homologous recombination directed genome editing in eukaryotic cells, such as human cells in a guide-independent fashion. Such an ANAGO induced homologous recombination directed genome/sequence editing (AISE) in human cells can be carried out precisely with no off-target events detected. Such a homologous donor nucleic acid can be either single-stranded or double-stranded. Such an AISE technology can be used in gene therapy, such as treating a disease, a disorder, or a condition treatable with genome editing in eukaryotic cells, for example, treating chronic myelogenous leukemia and lowering LDL levels in blood stream.

Some embodiments comprise a synthetic nucleic acid comprising: a first nucleic acid sequence comprising about 1000 or more contiguous nucleotides, or portion thereof, wherein the first nucleic acid sequence encodes an ANAGO that is a polypeptide, capable of editing a target nucleic acid sequence within a eukaryotic cell, wherein the ANAGO is a species-specific to the eukaryote; wherein the first nucleic acid sequence is modified from a second nucleic acid sequence of a microbial species; wherein the second nucleic acid sequence comprises a coding region that is capable of encoding a microbial Argonaute protein that has endonuclease activities in prokaryotic cells; wherein the first nucleic acid sequence is modified so that some of the microbial preferred codons of the second nucleic acid sequence are replaced with codons that have preferential usage by the target eukaryotic species.

Some embodiments comprise a synthetic nucleic acid comprising a first nucleic acid sequence comprising about 1000 or more contiguous nucleotides, or portion thereof having at least 70% identity to the nucleic acid sequence of SEQ ID NO:1; SEQ ID NO:2; SEQ ID NO:3, or SEQ ID NO:4, or portion thereof, wherein the first nucleic acid sequence encodes an ANAGO that is a polypeptide, capable of editing a target nucleic acid sequence within a human cell, wherein the ANAGO is a species-specific to the human being; wherein the first nucleic acid sequence is modified from a second nucleic acid sequence of a microbial species, and wherein the second nucleic acid sequence comprises a coding region that is capable of encoding a microbial Argonaute protein that has endonuclease activities in prokaryotic cells; and wherein the first nucleic acid sequence is modified so that the microbial preferred codons of the second nucleic acid sequence are replaced with codons that have preferential usage in the target human being.

Some embodiments comprise a synthetic nucleic acid comprising a first nucleic acid sequence comprising about 1000 or more contiguous nucleotides, wherein the first nucleic acid sequence encodes a codon-Adapted Nuclear Argonaute protein (ANAGO) that is a polypeptide, capable of editing a target nucleic acid sequence within a human cell in the presence of a donor nucleic acid without a guide nucleic acid, wherein the ANAGO is species-specific to the human, wherein the ANAGO is attached to a coding sequence of a nuclear localization signal (NLS) peptide; wherein the first nucleic acid sequence is produced by modifying a second nucleic acid sequence of a microbial species, and wherein the second nucleic acid sequence comprises a coding region that is capable of encoding a microbial Argonaute protein that has endonuclease activities in a microorganism; wherein the modifying comprises replacing microbial preferred codons of the second nucleic acid sequence with codons that have preferential usage in the human cell. In some embodiments, the first nucleic acid sequence comprises at least 85% identity to the nucleic acid sequence of SEQ ID NO:1; SEQ ID NO:2; SEQ ID NO:3, or SEQ ID NO:4. In some embodiments, the human cell is a human stem cell.

Some embodiments comprise a composition comprising a synthetic nucleic acid or an ANAGO described herein. In some embodiments, the composition is a pharmaceutical composition (e.g., a composition formulated for administration to a subject). In some embodiments, a pharmaceutical composition comprises one or more of a pharmaceutical acceptable excipient, diluent, additive or carrier.

Some embodiments comprise a composition comprising a synthetic nucleic acid described herein and a donor nucleic acid comprising: (i) a desired nucleic acid sequence to be introduced into a target sequence, wherein introducing the desired nucleic acid sequence is induced by the ANAGO; (ii) a 5′-flanking sequence; and (iii) a 3′-flanking sequence, wherein the 5′-flanking sequence and the 3′-flanking sequence independently comprise at least 10 consecutive nucleotides that are at least 90% identical to the target sequence located in the genome of a human cell. In some embodiments, the human cell is a human stem cell.

Some embodiments comprise a method of editing a genome of a eukaryotic cell comprising: introducing into the cell (i) a species-specific ANAGO encoded by the first synthetic nucleic acid sequence or an in vitro messenger RNA transcribed by the first synthetic nucleic acid sequence described herein; and (ii) a donor nucleic acid comprising: a desired nucleic acid sequence, a 5′-flanking sequence, and a 3′-flanking sequence, wherein each of the 5′-flanking sequence and the 3′-flanking sequence are located on opposite sides of the desired nucleic acid sequence and independently comprise at least 10 consecutive nucleotides that are at least 90% identical to a target sequence located in the genome of the eukaryotic cell.

Some embodiments comprise a method of editing a genome of a human cell comprising: introducing into the human cell (i) a human ANAGO encoded by the first synthetic nucleic acid sequence comprising about 1000 or more contiguous nucleotides, or portion thereof having at least 70% identity to the nucleic acid sequence of SEQ ID NO:1; SEQ ID NO:2; SEQ ID NO:3, or SEQ ID NO:4, or portion thereof; and (ii) a donor nucleic acid comprising: a desired nucleic acid sequence, a 5′-flanking sequence, and a 3′-flanking sequence, wherein each of the 5′-flanking sequence and the 3′-flanking sequence are located on opposite sides of the desired nucleic acid sequence and independently comprise at least 10 consecutive nucleotides that are at least 90% identical to a target sequence located in the genome of the human cell.

Some embodiments comprise a method of editing a genome of a human cell comprising: introducing into the human cell (i) a species-specific ANAGO encoded by the first nucleic acid sequence described herein, or an in vitro messenger RNA transcribed by the first nucleic acid sequence described herein; and (ii) a donor nucleic acid comprising: a desired nucleic acid sequence to be introduced into a target sequence, wherein introducing the desired nucleic acid sequence is induced by an ANAGO, a 5′-flanking sequence, and a 3′-flanking sequence, wherein the 5′-flanking sequence and the 3′-flanking sequence are located on opposite sides of the desired nucleic acid sequence and independently comprise at least 10 consecutive nucleotides that are at least 90% identical to the target sequence located in the genome of the human cell. In some embodiments, the first synthetic nucleic acid sequence comprises at least 85% identity to the nucleic acid sequence of SEQ ID NO:1, SEQ ID NO:2, SEQ ID NO:3, or SEQ ID NO:4. In some embodiments, the human cell is a human stem cell.

Some embodiments include an ANAGO induced gene editing method, wherein a single or multiple donor molecules targeting different sites can be introduced into a eukaryotic cell at same time for multiplex gene editing at same time.

Some embodiments comprise a kit comprising a synthetic nucleic acid, an ANAGO, a homologous donor nucleic acid, a composition described herein, or a combination thereof.

Some embodiments comprise a kit comprising an ANAGO, such as a human ANAGO, and a homologous donor nucleic acid described herein.

Some embodiments comprise an ANAGO that is further attached to a nuclear localization signal peptide sequence (NLS) to the N-terminus of the protein in ANAGO before introducing the ANAGO into the nucleus of mammalian cells. In some embodiments, a tag sequence such as human influenza hemagglutinin (HA) tag or his tag or myc tag is also included for protein detection purposes.

Some embodiments include delivery of an ANAGO into a eukaryotic cell, such as a human cell, wherein the ANAGO and the donor nucleic acid are introduced into the cell via viral vector, electroporation, lipofection, nucleofection, nanoparticle, or microinjection.

In some embodiments, an ANAGO, such as a human ANAGO, in a protein form, or an in vitro transcribed messenger RNA, is cloned into a mammalian expression vector before introduced into a mammalian cell. The mammalian expression vector can be a plasmid, a lentiviral vector, an adeno-associated viral vector, or any viral vector.

Some embodiments include the use of ANAGO Induced Sequence Editing (AISE) technology for efficient and precisely altering genes in human pluripotent stem cells (PSCs). The engineered ANAGO construct and a target specific donor molecule are delivered into human pluripotent stem cells (hPSCs) or cells of a specific tissue origin (e.g. human liver cancer cells and bone marrow Erythroid-myeloid precursors) via transfection.

Some embodiments include a method of editing a genome of a human stem cell comprising: introducing into the human stem cell (i) a species-specific ANAGO encoded by the first synthetic nucleic acid sequence described herein, or an in vitro messenger RNA transcribed by the first synthetic nucleic acid sequence of described herein; (ii) a donor nucleic acid comprising: a desired nucleic acid sequence, a 5′-flanking sequence, and a 3′-flanking sequence, wherein the 5′-flanking sequence and the 3′-flanking sequence are located on opposite sides of the desired nucleic acid sequence and independently comprise at least 10 consecutive nucleotides that are at least 90% identical to a target sequence located in the genome of the human stem cell; and (iii) a dominant negative form of human TP53 gene (P53DD) encoded the synthetic nucleic acid sequence (5′-GGATCCATGCCCCCAGGGAGCACTAAGCGAGCACTGCCCAACAACACCAGCTCCTCTCCCCAGCCAAAG AAGAAACCACTGGATGGAGAATATTTCACCCTTCAGATCCGTGGGCGTGAGCGCTTCGAGATGTTCCGA GAGCTGAATGAGGCCTTGGAACTCAAGGATGCCCAGGCTGGGAAGGAGCCAGGGGGGAGCAGGGCTC ACTCCAGCCACCTGAAGTCCAAAAAGGGTCAGTCTACCTCCCGCCATAAAAAACTCATGTTCAAGACAGA AGGGCCTGACTCAGACAAGCTT-3′ (SEQ ID NO: 45)) is expressed by a mammalian expression vector. In some embodiments, this method may increase the gene editing efficiency of the target sequence.

Some embodiments include a method of editing a genome of a human stem cell comprising: introducing into the human stem cell (i) a species-specific ANAGO encoded by the first synthetic nucleic acid sequence of described herein, or an in vitro messenger RNA transcribed by the first synthetic nucleic acid sequence of described herein; (ii) a donor nucleic acid comprising: a desired nucleic acid sequence, a 5′-flanking sequence, and a 3′-flanking sequence, wherein the 5′-flanking sequence and the 3′-flanking sequence are located on opposite sides of the desired nucleic acid sequence and independently comprise at least 10 consecutive nucleotides that are at least 90% identical to a target sequence located in the genome of the human stem cell; and (iii) a small interference RNA molecule target Human Rad51. In some embodiments, this method may increase the gene editing efficiency of the target sequence. Some embodiments include the use of the ANAGO induced gene editing technology in cancer immunotherapy, antiviral therapy, liver-targeted gene editing, and blindness treatment, etc. Some embodiments include the use of the ANAGO induced gene editing technology for treating diseases, disorder, or conditions that are potentially treatable using gene editing in eukaryotes, such as treating cancer, e.g. chronic myelogenous leukemia; treating cystic fibrosis and lowering LDL levels in blood stream; or use in hematopoietic stem cells (HSCs) therapy to replace defective bone marrow stem cells.

### BRIEF DESCRIPTION OF THE FIGURES

The drawings illustrate embodiments of the technology and are not limiting. For clarity and ease of illustration, the drawings are not made to scale, and, in some instances, various aspects may be shown exaggerated or enlarged to facilitate an understanding of particular embodiments.

FIG. 1a schematically depicts i) the knock-in of a 1.8 kb fragment of mCherry ORF flanked by homology arms into the exon 1 region of human PCSK9 gene; ii) the introduction of natural loss-of-function R104C-V114A mutation with a single stranded oligonucleotide 70mer donor molecule, for targeting PCSK9 gene by AISE approach; and iii) the introduction of loss-of-function Y142X-E144X mutation with a single-stranded oligonucleotide 89mer donor molecule, for targeting PCSK9 gene by AISE approach.

FIG. 1b shows genomic PCR screening to identify mCherry knock-in event.

FIG. 1c shows the homology-directed replacement (HDR) analysis of sequencing result of R104C-V114A 70mer donor replacement in HEK293 cells. FIG. 1c discloses SEQ ID NOS 28-38, respectively, in order of appearance.

FIG. 1d shows the homology-directed replacement (HDR) analysis of sequencing result of Y142X-E144X 89mer donor replacement in HEK293 cells. FIG. 1d discloses SEQ ID NOS 39-40, respectively, in order of appearance.

FIG. 2a schematically shows the introduction of a stop codon/HindIII compound mutation into the exon 6 of human ABL1 gene. The region of the engineered 6 specific base changes of BCR-ABL ex6 stop-H3 70mer are marked in light gray color.

FIG. 2b shows the homology directed replacement (HDR) analysis indicated that 43% genomic DNA PCR products have incorporated the stop codon/Hind III compound mutation in HEK293 cells transfected with the humanized NgAgo and the donor single-stranded oligo BCR-ABL ex6 stop-H3 70mer. FIG. 2b discloses SEQ ID NOS 41-43, respectively, in order of appearance.

FIG. 2c shows the sequencing result that shows the targeted base changes in the designated site (indicated by the dotted line). FIG. 2c discloses SEQ ID NO: 44.

FIG. 3a schematically shows the knock-in of mCherry reporter via micro homologous flanking arms to human Histone 2Bc gene by AISE technology as well as the relative positions of PCR primers used in the experiments.

FIG. 3b shows the 5′ junctional PCR product at expected size of 611 bp and 3′ junctional PCR product with expected size of 240 bp upon the precise insertion of mCherry in Histon 2Bc gene were visualized in electrophoresis.

FIG. 3c shows the fluorescence image of live HEK293 cells expressing Histone 2Bc-mCherry fusion protein 72 hours post transfection of human ANAGO derived from NgAgo and donor fragment.

FIG. 4a shows schematically the design of ANAGO directed gene editing with a donor oligo CD274-HDR1 72mer to replace the sequence near the 3′ end of exon 3, for precise gene inactivation.

FIG. 4b shows the graphic representation of ultra-deep sequencing result of PCR amplicon products for the target genomic region.

FIG. 4c shows the graphic representation of ultra-deep sequencing result of PCR amplicon products for the target genomic region when a guide molecule is also present.

FIG. 5a schematically shows the introduction of a donor molecule of BCR-ABL ex6 stop-H3 70mer with 5 specific base changes including introducing a stop codon (as a stop codon/Hind III compound mutation) into the exon 6 of human ABL1 gene in cultured human iPSCs using AISE technology. The region of the engineered 5 specific base changes of BCR-ABL ex6 stop-H3 70mer are marked in light gray color.

FIG. 5b shows the sequencing result of the targeted base changes in the designated sites (indicated by the dotted line in black color under the figure). FIG. 5b discloses SEQ ID NO: 46.

FIG. 6a schematically depicts i) the introduction of natural loss-of-function R104C-V114A mutation in human PSCs with a single stranded oligonucleotide 70mer donor molecule, for targeting PCSK9 gene using AISE technology; and ii) the introduction of loss-of-function Y142X-E144X mutation in human PSCs with a single-stranded oligonucleotide 89mer donor molecule, for targeting PCSK9 gene by AISE approach.

FIG. 6b shows schematically the introduction of a CD274ex3_stop mutation in human PSCs with a single-stranded oligonucleotide 72mer donor molecule, for targeting CD274 exon 3 by AISE approach.

FIG. 6c shows the graphic representation of ultra-deep sequencing result of PCR amplicon products for the target genomic region.

FIG. 6d shows the graphic representation of ultra-deep sequencing result of PCR amplicon products for the target genomic region.

### DETAILED DESCRIPTION

Presented herein, in some embodiments, is an ANAGO, compositions comprising an ANAGO, kits comprising an ANAGO, and uses thereof, for example, the use of the ANAGO in a homologous recombination directed genome editing in a subject, such as a eukaryote, e.g. a human being. The term “subject” refers to animals, typically mammalian animals, or plants. Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, and pigs) and experimental animal models (e.g., Drosophila, zebra fish, Xenopus, chick, mouse, rat, rabbit, guinea pig, and pig). In some embodiments a mammal is a human. A mammal can be any age or at any stage of development (e.g., an adult, teen, child, infant, or a mammal in utero). A mammal can be male or female. A mammal can be a pregnant female. In certain embodiments, a mammal can be an animal disease model. In some embodiments, the subject is human. In some embodiments, the subject is an animal. In some embodiments, the subject is a plant.

The term “ANAGO” refers to a codon-Adapted Nuclear Argonaute protein. ANAGO is an Argonaute protein variant derived from a microorganism having DNA endonuclease activities. The DNA coding sequences of a microbial Argonaute protein that has DNA endonuclease activities in microorganisms, such as but not limited to, Natronobacterium gregoryi [NgAgo], Pyrococcus furiosus [PfAgo], Thermus thermophilus [TtAgo], Methanocaldococcus jannaschii [MjAgo], Clostridium butyricum [CbAgo], or limnothrix rosea [LrAgo], etc., can be reengineered and adapted with the codons that are biasly used (with higher usage frequency) or have preferential usage in the cells of a eukaryotic species of interest to generate a new synthetic nucleic acid sequence that encodes an ANAGO, that is species-specific to the eukaryotic species. When expressed in a cell in the eukaryotic species, this ANAGO conserves and/or retains an ability to edit a target nucleic acid sequence within the eukaryotic cell. For example, when the preferentially used codons in human cells are used to replace the microbial preferred codons (referring herein to “reengineering” or “adapting”) of a DNA coding sequence of a microbial Argonaute protein to generate a new synthetic nucleic acid, which encodes an ANAGO, this type of ANAGO is called human ANAGO. Likewise, when the most frequently used codons or preferentially utilized codons in dog cells are used for reengineering a DNA coding sequence of microbial Argonaute protein to generate a new synthetic nucleic acid, which encodes an ANAGO, this type of ANAGO is called dog ANAGO. When the preferentially used codons in plants are used for reengineering a DNA coding sequence of a microbial Argonaute protein to generate a new synthetic nucleic acid, which encodes an ANAGO, this type of ANAGO is called plant ANAGO.

The term “ANAGO” can refer to a protein or to a nucleic acid encoding the protein. For example, when a messenger RNA comprising a synthetic nucleic acid sequence comprises a coding region that encodes an ANAGO in a protein form, the messenger RNA can be called “ANAGO RNA”. The DNA comprising the synthetic nucleic acid sequence that transcribes the ANAGO RNA can be called “ANAGO DNA”. For example, an ANAGO can be an in vitro transcribed messenger RNA. An ANAGO can be cloned into a mammalian expression vector before being introduced into a eukaryotic cell. For example, a human ANAGO in a form of a protein, or an in vitro transcribed messenger RNA, is cloned into a mammalian expression vector before being introduced into a human cell. The expression vector can be a plasmid, a lentiviral vector, an adeno-associated viral vector, or any viral vector.

The name ANAGO is used both in singular or plural with all letters always capitalized. For example, we say “an ANAGO”, or “two ANAGO”, “these ANAGO”, “a human ANAGO” or “two human ANAGO”, “an ANAGO is used”, or “two ANAGO are used”, and so on.

The Argonaute protein of a microorganism having DNA endonuclease activities (in short “endonuclease activities”) for generating an ANAGO can be from a thermo-bacterium that can tolerate a high temperature of 50-75° C. The ANAGO generated from such a thermo-bacterium can also tolerate a high temperature, such as 50° C., 55° C., >55° C., 50-55° C., 55-60° C., 60-65° C., 65-70° C., 70-75° C., 50-60° C., 60-70° C., 50-70° C., 50-75° C., or any temperature in a range bounded by any of the above values. The Argonaute protein of a microorganism having DNA endonuclease, from which an ANAGO can be derived, can be other microorganisms that are not listed herein.

The ANAGO described herein can be further attached to a Nuclear Localization Signal peptide sequence (NLS) to generate an ANAGO comprising a NLS. This ANAGO comprising a NLS can be further cloned into a eukaryotic expression vector, such as a mammalian expression vector to generate a recombinant ANAGO. This recombinant ANAGO, along with a gene specific donor, with or without a guide oligonucleotide molecule, can be introduced into eukaryotic cells, such as human cells, e.g. HEK293 cells to edit target genomic DNA in a homologous sequence-dependent manner in eukaryotes, such as human. The editing of genome in eukaryotes using species-specific ANAGO can be efficient. The efficiency of the editing of genome in eukaryotic cells, such as human cells, can be at least 1%, at least 5%, 1-60%, 5-60%, 1-70%, 5-70%, 1-80%, 5-80%, 1-90%, 5-90%, 1-100%, 5-100%, more than 1%, more than 5%, 5-10%, 10-15%, 15-20%, 20-25%, 25-30%, 30-35%, 35-40%, 40-45%, 45-50%, 10-20%, 20-30%, 30-40%, 40-50%, 50-60%, 60-70%, 70-80%, 80-90%, 90-100%, 7.1%, 6-8%, 40%, 43%, or any efficiency in a range bounded by any of the above values. The editing of genome in eukaryotic cells, such as human cells, using species-specific ANAGO, such as a human ANAGO can be precise with no significant random insertion and deletion detected. In some embodiments, the homologous recombination directed genome editing in eukaryotic cells, such as human cells, has no off-target events detected.

In some embodiments, the desired base changes can be precisely incorporated into the stem cell genomic DNA by employing ANAGO Induced Sequence Editing (AISE) technology. The gene editing in human stem cells using species-specific ANAGO can also be efficient. The efficiency of the desired editing of genome in eukaryotic cells, such as human stem cells, can be at least 0.1%, at least 0.5%, about 0.1-0.5%, about 0.1-0.2%, about 0.2-0.3%, about 0.3-0.5%, about 0.5-0.6%, about 0.6-0.7%, about 0.7-0.8%, about 0.5-1%, about 1-5%, at least 2%, at least 5%, about 2-5%, about 2-3%, about 3-4%, about 4-5%, about 5-6%, about 6-7%, about 5-7%, about 5-10%, about 1-60%, about 5-60%, about 1-70%, about 5-70%, about 1-80%, about 5-80%, about 1-90%, about 5-90%, about 1-100%, more than 1%, more than 2%, more than 5%, about 5-10%, about 10-15%, about 15-20%, about 20-25%, about 25-30%, about 30-35%, about 35-40%, about 40-45%, about 45-50%, about 10-20%, about 20-30%, about 30-40%, about 40-50%, about 50-60%, about 60-70%, about 70-80%, about 80-90%, about 90-100%, about 0.5-1%, about 5-7%, about 15-17%, about 20-21%, about 0.23%, about 2.9%, about 6%, about 16.5%, about 20.5%, or any efficiency in a range bounded by any of the above values. In some embodiments, the unintended indel events commonly associated with a DNA break can be very low, such as less than 1%, less than 2%, less than 3%, less than 4%, about 0-0.1%, about 0.1-1%, about 1-2%, about 2-3%, about 3-4%, or about 4%.

In some embodiments, a donor molecule derived from a target gene may contain nucleotides that are changed involving both deletion and replacement or conversion. In this case, the gene editing employed AISE technology may result in precisely deleting target nucleotides and insertion of replacement nucleotides at the target region in cultured human iPSCs. The efficiency of deletion and insertion may be same, similar, or with small difference. The efficiency of having both deletion and insertion together may be both about 2-3%.

In some embodiments, the AISE technology described herein may be used to edit a single base change of human genome in human cells, such as stem cells. In some embodiments, the AISE technology described herein may be used to generate discrete point mutations that mimic natural mutation of a target gene in human PSCs, with single base change at each of the multiple positions of a donor molecule. For example, two altered nucleotides may be located at the 20th or 40th of a 70mer ssDNA donor molecule. The gene editing at multiple locations may be efficient, and their editing efficiencies may be same, similar, or different, with each efficiency independently about 10-20%, 20-30%, 10-15%, 15-20%, 20-25%, 25-30%, 16-17%, 20-21%, 30-40%, 40-50%, 50-60%, 60-70%, 70-80%, 80-90%, or 90-100%, such as 16.5%, or 20.5%. The length of short arm of homologous sequence in the donor molecule may be 18-40 nts, 18-19 nts, 19 nts, 19-20 nts, 20-25 nts, 25-30 nts, 30 nts, 30-35 nts, 35-40 nts, 40-50 nts, 50 nts or longer. In some embodiments, the longer arm of homologous sequence in a donor molecule may lead to higher gene editing efficiency. In some embodiments, the longer arm of homologous sequence close to a single base change in a donor molecule may lead to higher gene editing efficiency of the discrete point mutation than the shorter arm of homologous sequence close to the other single base change in the same donor molecule. For example, an arm of 30 nts homologous sequence in a donor molecule may result in 20-21% gene editing efficiency, while an arm of 19 nts homologous sequence in a donor molecule may only lead to 16-17% gene editing efficiency. In some embodiments, a short homologous region of 19 bases on each side of modified bases may be sufficient to lead to an intended homology directed recombination (HDR).

The fact that the AISE technology described herein can be used to edit a single base change of human genome in human cells, such as stem cells, is of great significance. The functional features of a protein are affected and determined not only by its primary sequence made of the amino acids, but also by its secondary structure such as its protein folding. Thus, with a single codon change of a gene that does not lead to the change of the amino acid identity, but the resulting protein may fold differently from the protein of the parent gene coded for. A synonymous substitution or synonymous mutation is the evolutionary substitution of one base for another in an exon of a gene coding for a protein, such that the produced amino acid sequence is not altered. It is well known that a synonymous mutation can affect transcription, splicing, mRNA transport, and translation, any of which could alter the resulting phenotype, rendering the synonymous mutation non-silent. The substrate specificity of the tRNA to the rare codon can affect the timing of translation, and in turn the co-translational folding of the protein. This is reflected in the codon usage bias that is observed in many species. There are many examples by which synonymous mutations (with only a single base change) contribute to gene expression and disease phenotype. Some examples of Synonymous single nucleotide polymorphisms (sSNPs) (with change only codons, not amino acids) associated with changes in mRNA and protein binding are listed herein. Amyotropic lateral sclerosis (ALS) is a progressive, lethal, neurodegenerative disorder associated with mutations in the SOD1 gene. Numerous missense mutations and a number of synonymous mutations such as Gly10-GGC>GGT, Ser59-AGT>AGC, Ala140-GCT>GCA, Thr116-ACA>ACG, Asn139-AAC>AAT, and Gln153-CAA>CAG have also been reported in SOD1. Some examples of sSNP-associated splicing defects are listed herein. Charcot-Marie-Tooth disease type 1B (CMT1B) is caused by mutations in the gene encoding myelin protein zero. In a familial case of late onset CMT1B a synonymous mutation in exon three, Val102-GTG>GTA, renders the binding site for a small nuclear RNA (snRNA U1) enhanced, causing truncation of exon three. Another report concerns the X-linked metabolic disorder caused by pyruvate dehydrogenase (PDH)-Ela deficiency. In a severe case of familial Ela deficiency, a synonymous mutation Gly185-GGA>GGG caused aberrant splicing in a subset of cases, resulting in the skipping of exon six. Exon six contains a thiamine pyrophosphate binding site necessary for proper enzyme function. Therefore, skipping this exon results in a dysfunctional protein. Therefore, the use of the AISE technology that enables to do the precise gene editing of a single nucleotide at a target position in human stem cells offers large flexibility in gene therapy and opens up a door for a new approach to treat gene related diseases and disorders with precision.

In some embodiments, AISE technology described herein may be used to simultaneously edit two or more unrelated genes in the genome of human PSC. For example, ANAGO expression construct may be transiently co-transfected with two or more different donor molecules or templates targeting two or more different genes. The gene editing efficiencies of the different unrelated genes may be same, similar, or different, such as independently in a range of 0.1-10%, 0.2-10%, 0.1-0.5%, 0.2-0.5%, 0.5-1%, 1-5%, 5-10%, 0.5-10%, 0.5-20%, 0.5-30%, 05-40%, 0.5-50%, 0.5-60%, 0.5-70%, 0.5-80%, 0.5-90%, or 0.5-100%. In some embodiments, the editing efficiency for one gene is about 5-7%, 6%, 5-10%, 10-15%, 15-20%, 20-30%, 30-40%, 40-50%, 50-60%, 60-80%, or 80-100%; and the editing efficiency for the other gene is about 0.1-1%, 0.1-0.5%, 0.1-0.2%, 0.2-0.3%, 0.3-0.5%, 0.5-0.7%, 0.7%, 0.5-1%, 1-5%, 5-10%, 10-20%, 20-30%, 30-40%, or 40-50%.

Either double-stranded or single strand DNA molecules can be used as donor molecules or templates. The homologous donor molecule can have a sequence either short or long. The length of a homology flanking region or arm (5′ and/or 3′) can be as short as 10 nucleotides. The length of a homology arm or flanking region can be as long as 500 or more nucleotides without upper limit. For example, the length of a homology arm or flanking can be 10-500 nucleotides, 20 nucleotides, 10-20 nucleotides, 20-50 nucleotides, 30-100 nucleotides, 50-100 nucleotides, 100-150 nucleotides, 150-200 nucleotides, 200-300 nucleotides, 300-400 nucleotides, 400-500 nucleotides, 400-600 nucleotides, 500-600 nucleotides, 600-700 nucleotides, 100-300 nucleotides, 300-500 nucleotides, 500-700 nucleotides, 700-1000 nucleotides, 150-250 nucleotides, 250-350 nucleotides, 350-450 nucleotides, 450-550 nucleotides, 550-650 nucleotides, 700-800 nucleotides, 800-900 nucleotides, 900-1000 nucleotides, 800-1000 nucleotides, or any number of nucleotides in a range bounded by any of the above values.

In some embodiments, an altered DNA sequence can be embedded between 5′- and 3′-flanking homologous arms. The length of an altered DNA sequence can be as short as a single nucleotide or as long as 1200 or more nucleotides.

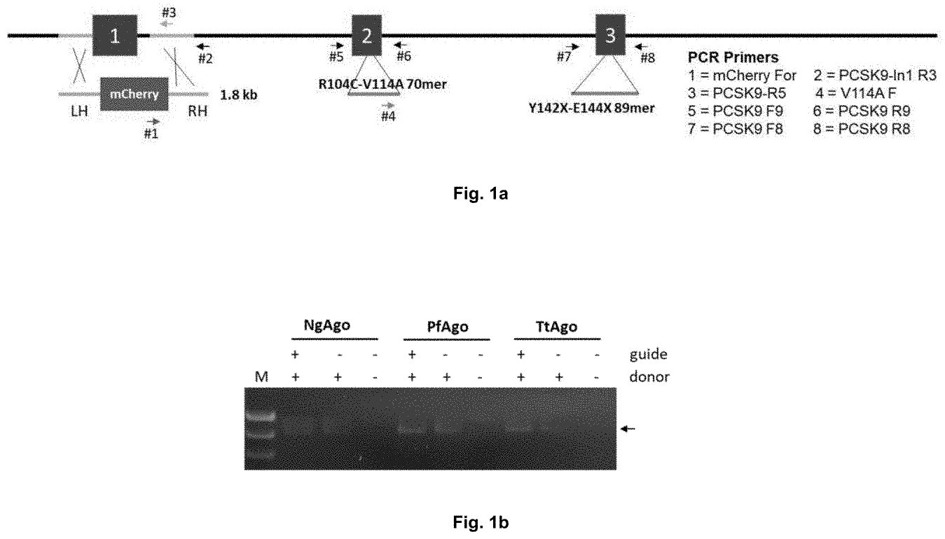

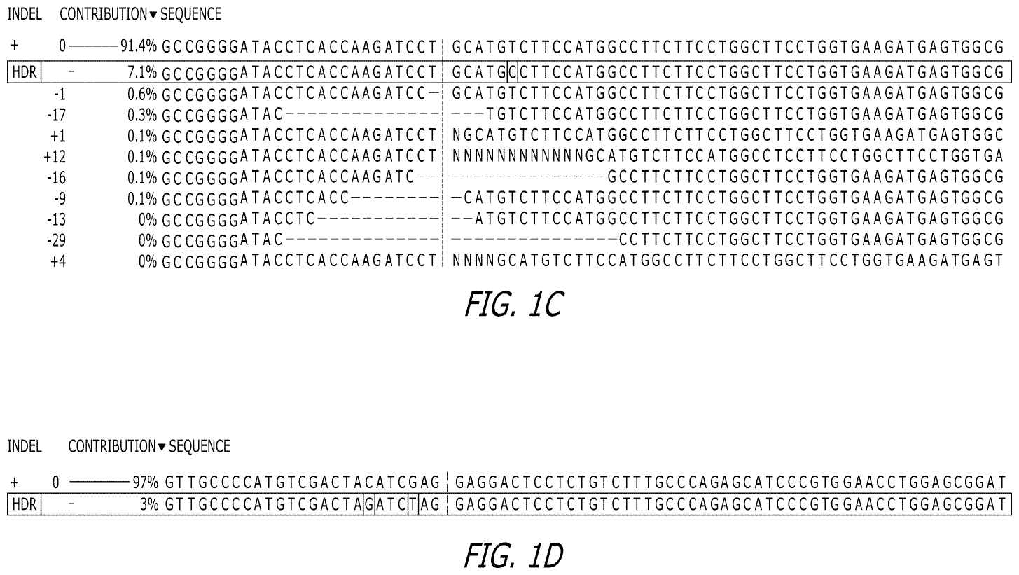

In some embodiments, a single-stranded oligonucleotide (e.g. PCSK9 R104C-V114A 70mer 5′-CCTGCAGGCCCAGGCTGCC [symbol] GCCGGGGATACCTCACCAAGATCCTGCATG [symbol] CTTCCATGGCCTTCTTCCT-3′ (SEQ ID NO: 5), FIG. 1a) can be chemically synthesized and used as a donor molecule. The donor molecule harbors 1 or multiple base mismatches (boxed bases) to the targeted genomic sequence at a position at least 15 bases away from both the 5′ and 3′ ends of oligo.

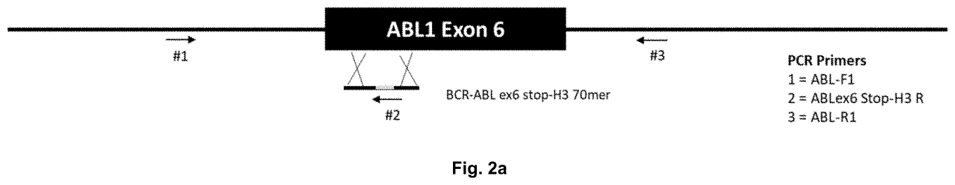

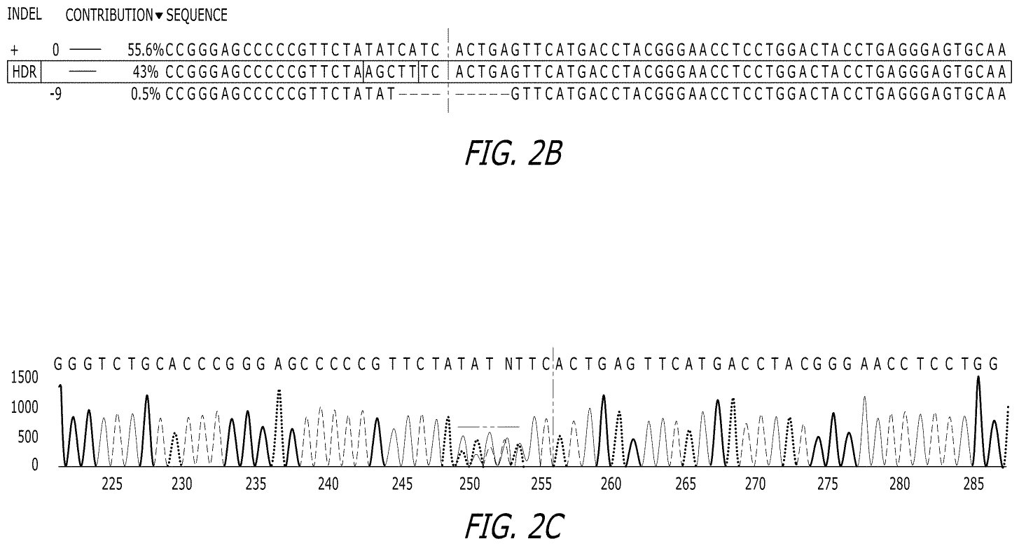

In some embodiments, a single-stranded oligonucleotide (e.g. BCR-ABLex6 stop-H3 70mer 5′-GGCAGGGGTCTGCACCCGGGAGCCCCCGTTCT [symbol] TCACTGAGTTCATGACCTACGGGAACCTCCTG-3′ (SEQ ID NO: 6), FIG. 2a) can be chemically synthesized and used as a donor molecule. The donor molecule can harbor a restriction enzyme recognition site (boxed sequence) that is inserted into the targeted site and also introduces an in-frame premature stop codon (TAA, bold faced).

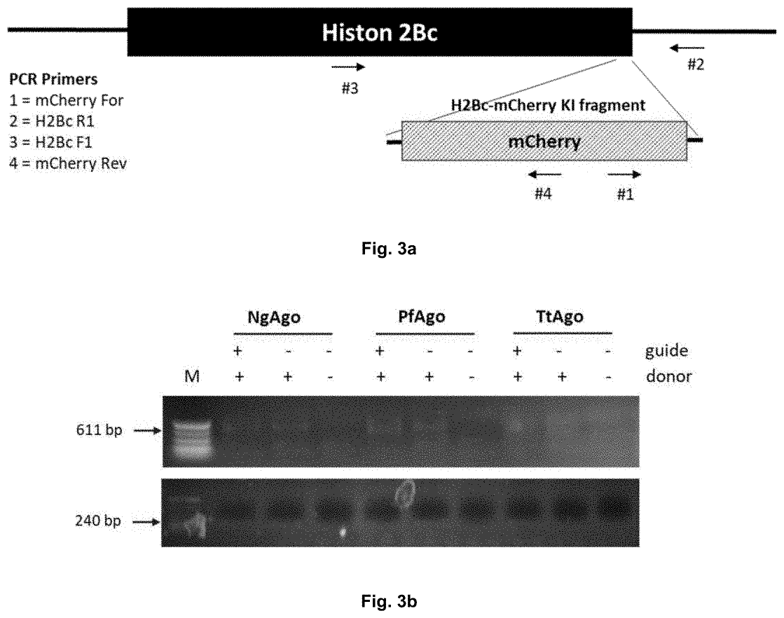

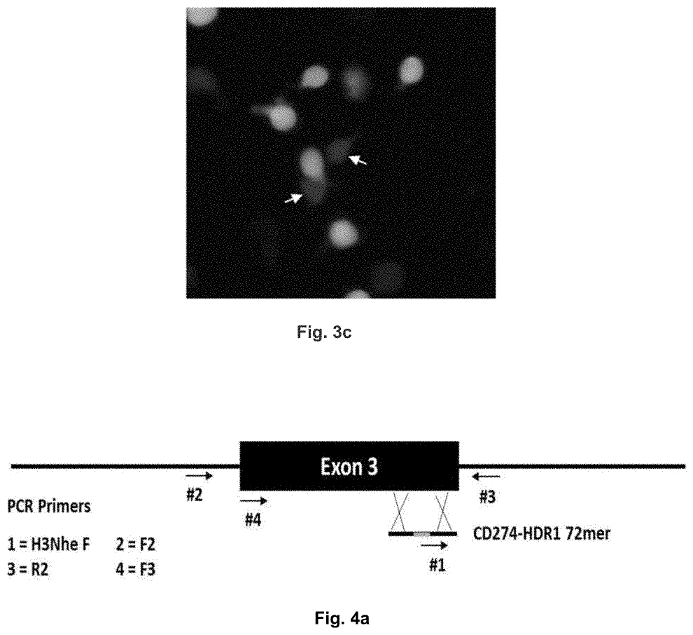

In some embodiments, an over 800 bps long donor DNA fragment (e.g. H2Bc-mCherry KI fragment, FIG. 3a) can be synthesized by using a pair of PCR primers that flanks both ends of a long exogenous gene fragment with a short stretch of sequence (15 to 50 bases or longer) that is homology to the target site in the genome (e.g. the C terminal coding region of human Histon 2Bc gene, FIG. 3a).

In some embodiments, a double-stranded large donor DNA fragment can be generated via fusion PCR (e.g. the 1.8 kb of PCSK9-mCherry knock-in fragment to replace the exon 1 sequence of human PCSK9 gene, see FIG. 1a). The left and right homology arms (over 500 bp long for each one) are generated and fused to an exogenous sequence such as mCherry fragment. The left and right homology regions can be separated by a sequence of 0 to at least 648 bps.

The term “nucleic acid” refers to one or more nucleic acids (e.g., a set or subset of nucleic acids) of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. In some embodiments a nucleic acid refers to DNA. In some embodiments a nucleic acid refers to RNA. Unless specifically limited, the term encompasses nucleic acids comprising deoxyribonucleotides, ribonucleotides and known analogs of natural nucleotides. A nucleic acid may include, as equivalents, derivatives, or variants thereof, suitable analogs of RNA or DNA synthesized from nucleotide analogs, single-stranded (“sense” or “antisense”, “plus” strand or “minus” strand, “forward” reading frame or “reverse” reading frame) and double-stranded polynucleotides. Nucleic acids may be single or double stranded. A nucleic acid can be of any length of 2 or more, 3 or more, 4 or more, or 5 or more contiguous nucleotides. A nucleic acid can comprise a specific 5′ to 3′ order of nucleotides known in the art as a sequence (e.g., a nucleic acid sequence, e.g., a sequence).

A nucleic acid may be naturally occurring and/or may be synthesized, copied or altered (e.g., by a technician, scientist or one of skill in the art). For, example, a nucleic acid may be an amplicon. A nucleic acid may be from a nucleic acid library, such as a gDNA, cDNA or RNA library, for example. A nucleic acid can be synthesized (e.g., chemically synthesized) or generated (e.g., by polymerase extension in vitro, e.g., by amplification, e.g., by PCR). A nucleic acid may be, or may be from, a plasmid, phage, virus, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. Nucleic acid provided for processes or methods described herein may comprise nucleic acids comprising 1 to 1000 or more, 1 to 1000, 1 to 500, 1 to 200, 1 to 100, 1 to 50, 1 to 20, or 1 to 10 nucleotides in length. Oligonucleotides are relatively short nucleic acids. Oligonucleotides can be from about 2 to 200, 2 to 150, 2 to 100, 2 to 50, or 2 to about 35 nucleotides in length. In certain embodiments, oligonucleotides are 18 to 30, 20 to 28 or 21-26 nucleotides in length. In some embodiments, oligonucleotides are single stranded. In certain embodiments, oligonucleotides are primers. Primers are often configured to hybridize to a selected complementary nucleic acid and are configured to be extended by a polymerase after hybridizing.

A genome refers to the genetic material of a cell, a virus, or an organism. The genetic material of a cell or organism often comprises one or more genes. In certain embodiments a gene comprises or consists of one or more nucleic acids. The term “gene” means the segment of DNA involved in producing a polypeptide chain and can include coding regions (e.g., exons), regions preceding and following the coding region (leader and trailer) involved in the transcription/translation of the gene product and the regulation of the transcription/translation, as well as intervening sequences (introns) between individual coding segments (exons). A gene may not necessarily produce a peptide or may produce a truncated or non-functional protein due to genetic variation in a gene sequence (e.g., mutations in coding and non-coding portions of a gene). For example, a non-functional gene can be a pseudogene. A gene may also produce a non-coding RNA, such as long non-coding RNA (IncRNA); microRNA (miRNA); small interfering RNA (siRNA); Piwi-interacting RNA (piRNAs); or small nucleolar RNA (snoRNA), and other short RNA (Ma L, Bajic V B, and Zhang Z, “On the classification of long non-coding RNAs”, RNA Biology. 10 (6): 925-33, June 2013). A gene, whether functional or non-functional, can often be identified by homology to a gene in a reference genome. For example, any specific gene (e.g., a gene of interest, a counterpart gene, a pseudogene and the like) of a subject can be identified in another subject, genome or in a reference genome by one of skill in the art. In a diploid subject, a gene often comprises a pair of alleles (e.g., two alleles). Thus, a method, system or process herein can be applied to one or both alleles of a gene. In some embodiments a method, system or process herein is applied to each allele of a gene.

The term “percent identical” or “percent identity” refers to sequence identity between two amino acid sequences. Identity can be determined by comparing a position in each sequence which may be aligned for purposes of comparison. When an equivalent position in the compared sequences is occupied by the same amino acid, then the molecules are identical at that position. When the equivalent site is occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. In some embodiments an alignment program that permits gaps in the sequence is utilized to align the sequences. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches; and is especially tolerant of small gaps and nucleotide sequence errors. Nucleic acid-encoded amino acid sequences can be used to search both protein and DNA databases.

In some embodiments a nucleic acid described herein comprises a label. As used herein, the terms “label” or “labeled” refers to incorporation of a detectable marker, e.g., by incorporation of a radiolabeled amino acid or attachment to a polypeptide of biotin moieties that can be detected by marked avidin (e.g., streptavidin containing a fluorescent marker or enzymatic activity that can be detected by optical or colorimetric methods). In certain embodiments, the label or marker can also be therapeutic. Various methods of labeling polypeptides and glycoproteins can be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance.

In some embodiments, a carrier, radioisotope and/or a polypeptide can be indirectly or directly associated with, or bound to (e.g., covalently bound to, or conjugated to), a nucleic acid described herein. In certain embodiments agents or molecules are sometimes conjugated to or bound to nucleic acids to alter or extend the in vivo half-life of a nucleic acid or fragment thereof. In some embodiments, a nucleic acid described herein is fused or associated with one or more polypeptides (e.g., a toxin, ligand, receptor, cytokine, antibody, the like or combinations thereof). In certain embodiments, a nucleic acid described herein is linked to a half-life extending vehicle known in the art. Such vehicles include, but are not limited to, polyethylene glycol, glycogen (e.g., glycosylation of the antigen binding protein), and dextran. Such vehicles are described, e.g., in U.S. application Ser. No. 09/428,082, now U.S. Pat. No. 6,660,843 and published PCT Application No. WO 99/25044, hereby incorporated by reference.

In some embodiments carriers or anti-bacterial medications are bound to a nucleic acid described herein by a linker. A linker can provide a mechanism for covalently attaching a carrier and/or anti-bacterial medications to a nucleic acid described herein. Any suitable linker can be used in a composition or method described herein. Non-limiting examples of suitable linkers include silanes, thiols, phosphonic acid, and polyethylene glycol (PEG). Methods of attaching two or more molecules using a linker are well known in the art and are sometimes referred to as “crosslinking”. Non-limiting examples of crosslinking include an amine reacting with a N-hydroxysuccinimide (NHS) ester, an imidoester, a pentafluorophenyl (PFP) ester, a hydroxymethyl phosphine, an oxirane or any other carbonyl compound; a carboxyl reacting with a carbodiimide; a sulfhydryl reacting with a maleimide, a haloacetyl, a pyridyldisulfide, and/or a vinyl sulfone; an aldehyde reacting with a hydrazine; any non-selective group reacting with diazirine and/or aryl azide; a hydroxyl reacting with isocyanate; a hydroxylamine reacting with a carbonyl compound; the like and combinations thereof.

In certain embodiments, presented herein is a nucleic acid that encodes and/or expresses an Argonaute protein or Argonaute polypeptide or a functional fragment thereof. An Argonaute protein or Argonaute polypeptide is a DNA-guided endonuclease that can edit nucleic acids within a cell or subject (e.g., within a genome of a subject) in a target specific manner.

In some embodiments, an ANAGO comprises a polypeptide encoded by the sequence of SEQ ID NO:1, SEQ ID NO:2, SEQ ID NO:3, or SEQ ID NO:4. In certain embodiments, an ANAGO comprises an amino acid sequence having 70% to 100% identity, 80% to 100% identity, 90% to 100% identity or 95% to 100% identity, 70-80% identity, 80-90% identity, 90-100% identity, 70-75% identity, 75-80% identity, 80-85% identity, 85-90% identity, 90-95% identity, 95-100% identity, at least 70% identity, at least 75% identity, at least 80% identity, at least 85% identity, at least 90% identity, at least 95% identity, at least 98% identity, at least 99%, or 100% identity to a polypeptide encoded by the sequence of SEQ ID NO:1, SEQ ID NO:2, SEQ ID NO:3, or SEQ ID NO:4, or a portion thereof. In certain embodiments, an ANAGO conserves and/or retains an ability to edit a target nucleic acid sequence (e.g., RNA, DNA, a gene, promoter or the like) within a eukaryotic cell (e.g., a cell of a subject) in a target specific manner. The ability to edit a target sequence within a genome of a subject refers to an ability to insert, remove and/or replace one or more specific nucleotides within a target sequence of a cell (e.g., a human cell).

Argonaute (Ago) proteins are small RNA or DNA guided, site-specific endonucleases, which are present in all three kingdoms of life. The various functions of Argonaute proteins have been studied extensively. Recent studies have suggested that prokaryotic Argonautes are involved in identifying foreign genetic elements in a sequence specific manner and/or in the recruitment of nucleases. Many DNA coding sequences of prokaryotic Argonautes proteins, such as NgAgo, PfAgo, TtAgo, MjAgo, CbAgo, or LrAgo, etc. can be reengineered and adapted to generate an ANAGO that is species-specific in a eukaryote. The microbial preferred codons can be changed to human preferred codons, such as a codon with a frequency of at least 1 per thousand, at least 2 per thousand, at least 3 per thousand, at least 4 per thousand, at least 5 per thousand, at least 6 per thousand, at least 7 per thousand, at least 8 per thousand, at least 9 per thousand, at least 10 per thousand, at least 11 per thousand, at least 12 per thousand, at least 13 per thousand, at least 14 per thousand, at least 15 per thousand, at least 16 per thousand, at least 17 per thousand, at least 18 per thousand, at least 19 per thousand, at least 20 per thousand, at least 21 per thousand, at least 22 per thousand, at least 23 per thousand, at least 24 per thousand, at least 25 per thousand, at least 26 per thousand, at least 27 per thousand, at least 28 per thousand, at least 29 per thousand, at least or 30 per thousand, at least 31 per thousand, at least 32 per thousand, at least 33 per thousand, at least 34 per thousand, at least 35 per thousand, at least 36 per thousand, at least 37 per thousand, at least 38 per thousand, at least 39 per thousand, at least 40 per thousand, to generate a human ANAGO.

Examples of microbial preferred codons are: ACG, CCG, UCG, GUA, CUA, UUA, and GCG etc.; or their corresponding DNA codons.

Examples of human preferred codons include, but are not limited to, UUU, UCU, UAU, UGU, UUC, UCC, UAC, UGC, UUA, UCA, UUG, UCG, UGG, CUU, CCU, CAU, CGU, CUC, CCC, CAC, CGC, CUA, CCA, CAA, CGA, CUG, CCG, CAG, CGG, AUU, ACU, AAU, AGU, AUC, ACC, AAC, AGC, AUA, ACA, AAA, AGA, AUG, ACG, AAG, AGG, GUU, GCU, GAU, GGU, GUC, GCC, GAC, GGC, GUA, GCA, GAA, GGA, GUG, GCG, GAG, GGG, etc. or their corresponding DNA codons.

The frequency per thousand of the human preferred codons and their corresponding amino acids are listed in Table A.

TABLE A
Homo Sapiens Codon Usage*

| codon | AA | FPT | codon | AA | FPT | codon | AA | FPT | codon | AA | FPT |
|---|---|---|---|---|---|---|---|---|---|---|---|
| UUU | F | 17.6 | UCU | S | 15.2 | UAU | Y | 12.2 | UGU | C | 10.6 |
| UUC | F | 20.3 | UCC | S | 17.7 | UAC | Y | 15.3 | UGC | C | 12.6 |
| UUA | L | 7.7 | UCA | S | 12.2 | UAA | Stop | 1 | UGA | Stop | 1.6 |
| UUG | L | 12.9 | UCA | S | 4.4 | UAG | Stop | 0.8 | UGG | W | 13.2 |
| CUU | L | 13.2 | CCU | P | 17.5 | CAU | H | 10.9 | CGU | R | 4.5 |
| CUC | L | 19.6 | CCC | P | 19.8 | CAC | H | 15.1 | CGC | R | 10.4 |
| CUA | L | 7.2 | CCA | P | 16.9 | CAA | Q | 12.3 | CGA | R | 6.2 |
| CUG | L | 39.6 | CCG | P | 6.9 | CAG | Q | 34.2 | CGG | R | 11.4 |
| AUU | I | 16 | ACU | T | 13.1 | AAU | N | 17 | AGU | S | 12.1 |
| AUC | I | 20.8 | ACC | T | 18.9 | AAC | N | 19.1 | AGC | S | 19.5 |
| AUA | I | 7.5 | ACA | T | 15.1 | AAA | K | 24.4 | AGA | R | 12.2 |
| AUG | M | 22 | ACG | T | 6.1 | AAG | K | 31.9 | AGG | R | 12 |
| GUU | V | 11 | GCU | A | 18.4 | GAU | D | 21.8 | GGU | G | 10.8 |
| GUC | V | 14.5 | GCC | A | 27.7 | GAC | D | 25.1 | GGC | G | 22.2 |
| GUA | V | 7.1 | GCA | A | 15.8 | GAA | E | 29 | GGA | G | 16.5 |
| GUG | V | 28.1 | GCG | A | 7.4 | GAG | E | 39.6 | GGG | G | 16.5 |

*Note:
40662582 codons (RNA) of Homo sapiens were analyzed with the Kazusa DNA Research Institute Website (www.kazusa.or.jp/codon/);
AA: single letter amino acid code;
FPT: frequency per 1000 codons.

The frequency of preferred codon usage in human genes relating to the corresponding amino acid, based on a free software SnapGene Viewer, are listed in Table B. The ones highlighted in bold are the ones most frequently used by humans.

TABLE B
Homo sapiens Preferred Codon Usage Frequency*

| Codon | AA | F | Codon | AA | F | Codon | AA | F | Codon | AA | F |
|---|---|---|---|---|---|---|---|---|---|---|---|
| TTT | Phe | 0.46 | TCT | Ser | 0.19 | TAT | Tyr | 0.44 | TGT | Cys | 0.46 |
| TTC | Phe | 0.54 | TCC | Ser | 0.22 | TAC | Tyr | 0.56 | TGC | Cys | 0.54 |
| TTA | Leu | 0.08 | TCA | Ser | 0.15 | TAA | Stop | 0.30 | TGA | Stop | 0.47 |
| TTG | Leu | 0.13 | TCG | Ser | 0.05 | TAG | Stop | 0.24 | TGG | Trp | 1.00 |
| CTT | Leu | 0.13 | CCT | Pro | 0.29 | CAT | His | 0.42 | CGT | Arg | 0.08 |
| CTC | Leu | 0.20 | CCC | Pro | 0.32 | CAC | His | 0.58 | CGC | Arg | 0.18 |
| CTA | Leu | 0.07 | CCA | Pro | 0.28 | CAA | Gin | 0.27 | CGA | Arg | 0.11 |
| CTG | Leu | 0.40 | CCG | Pro | 0.11 | CAG | Gln | 0.73 | CGG | Arg | 0.2 |
| ATT | Ile | 0.36 | ACT | Thr | 0.25 | AAT | Asn | 0.47 | AGT | Ser | 0.15 |
| ATC | Ile | 0.47 | ACC | Thr | 0.36 | AAC | Asn | 0.53 | AGC | Ser | 0.24 |
| ATA | Ile | 0.17 | ACA | Thr | 0.28 | AAA | Lys | 0.43 | AGA | Arg | 0.21 |
| ATG | Met | 1.00 | ACG | Thr | 0.11 | AAG | Lys | 0.57 | AGG | Arg | 0.21 |
| GTT | Val | 0.18 | GCT | Ala | 0.27 | GAT | Asp | 0.46 | GGT | Gly | 0.16 |
| GTC | Val | 0.24 | GCC | Ala | 0.40 | GAC | Asp | 0.54 | GGC | Gly | 0.34 |
| GTA | Val | 0.12 | GCA | Ala | 0.23 | GAA | Glu | 0.42 | GGA | Gly | 0.25 |
| GTG | Val | 0.46 | GCG | Ala | 0.11 | GAG | Glu | 0.58 | GGG | Gly | 0.25 |

*Note:
Codon: DNA codon;
AA: three- etter amino acid code;
F: Frequency;
Website for SnapGene Viewer: https://www.snapgene.com/snapgene-viewer/;
the codons that highlighted in bold are the ones more preferentially used in human.

Any of the microbial preferred codons in the nucleic acid coding sequences of a microbial Argonaute protein can be replaced by any of the human preferred codons during the reengineering process described herein to generate a synthetic nucleic acid that encodes an ANAGO. In some embodiments, at least about 30%, at least about 40%, at least about 50%, at least about 60%, at least about 70%, at least about 80%, at least about 85%, at least about 90%, at least about 95%, about 100%, about 30-50%, about 50-70%; about 70-80%, about 80-90%, about 90-100%, abut 80-85%, about 85-90%, about 90-95%, about 95-100%, or any percentage in a range bonded by these values of the microbial preferred codons in the nucleic acid sequence of the microbial species are replaced by the human codons to generate a synthetic nucleic acid that encodes an ANAGO. In some embodiments, about at least 30, at least 50, at least 70, at least 100, at least 200, at least 300, at least 400, at least 500, at least 600, at least 700, about 30-1000, about 30-50, about 50-70, about 70-90, about 90-100, about 50-60, about 60-70, about 70-80, about 80-90, about 100-300, about 300-500, about 500-700, about 700-900, about 900-1000, about 100-200, about 200-300, about 300-400, about 400-500, about 500-600, about 600-700, about 700-800, about 800-900, about 900-1000, about 1000 or more of bases of the microbial preferred codons in the nucleic acid sequence of the microbial species are replaced by the human codons to generate a synthetic nucleic acid that encodes an ANAGO. In some embodiments, the ANAGO coding sequence shares about 60%, about 70%, about 80%, about 81%, about 85%, about 90%, about 60-90%, about 60-70%, about 70-80%, about 80-85%, about 80-90%, or any percentage in a range bounded by these values of identity with the microbial version of the Argonaute coding sequence from which the ANAGO is derived.

In some embodiments, at least 70%, at least 80%, at least 90%, at least 95%, at least 99%, or 100% of the ACG codons in the nucleic acid coding sequence of a microbial Argonaute protein are replaced with a human preferred codon. In some embodiments, at least 70%, at least 80%, at least 90%, at least 95%, at least 99%, or 100% of the ACG codons in the nucleic acid coding sequence of a microbial Argonaute protein codons in the nucleic acid coding sequence of a microbial Argonaute protein are replaced with a UUU codon. In some embodiments, at least 70%, at least 80%, at least 90%, at least 95%, at least 99%, or 100% of the ACG codons in the nucleic acid coding sequence of a microbial Argonaute protein codons in the nucleic acid coding sequence of a microbial Argonaute protein are replaced with a UCU codon. In some embodiments, at least 70%, at least 80%, at least 90%, at least 95%, at least 99%, or 100% of the ACG codons in the nucleic acid coding sequence of a microbial Argonaute protein are replaced with a UAU codon. In some embodiments, at least 70%, at least 80%, at least 90%, at least 95%, at least 99%, or 100% of the ACG codons in the nucleic acid coding sequence of a microbial Argonaute protein are replaced with a UGU codon. In some embodiments, at least 70%, at least 80%, at least 90%, at least 95%, at least 99%, or 100%, of the ACG codons in the nucleic acid coding sequence of a microbial Argonaute protein are replaced with a UUC codon. In some embodiments, at least 70%, at least 80%, at least 90%, at least 95%, at least 99%, or 100%, of the ACG codons in the nucleic acid coding sequence of a microbial Argonaute protein are replaced with a UCC codon. In some embodiments, at least 70%, at least 80%, at least 90%, at least 95%, at least 99%, or 100%, of the ACG codons in the nucleic acid coding sequence of a microbial Argonaute protein are replaced with a UAC codon. In some embodiments, at least 70%, at least 80%, at least 90%, at least 95%, at least 99%, or 100%, of the ACG codons in the nucleic acid coding sequence of a microbial Argonaute protein are replaced with a UGC codon. In some embodiments, at least 70%, at least 80%, at least 90%, at least 95%, at least 99%, or 100%, of the ACG codons in the nucleic acid coding sequence of a microbial Argonaute protein are replaced with a UUA codon. In some embodiments, at least 70%, at least 80%, at least 90%, at least 95%, at least 99%, or 100%, of the ACG codons in the nucleic acid coding sequence of a microbial Argonaute protein are replaced with a UCA codon. In some embodiments, at least 70%, at least 80%, at least 90%, at least 95%, at least 99%, or 100%, of the ACG codons in the nucleic acid coding sequence of a microbial Argonaute protein are replaced with a UUG codon. In some embodiments, at least 70%, at least 80%, at least 90%, at least 95%, at least 99%, or 100%, of the ACG codons in the nucleic acid coding sequence of a microbial Argonaute protein are replaced with a UCG codon. In some embodiments, at least 70%, at least 80%, at least 90%, at least 95%, at least 99%, or 100%, of the ACG codons in the nucleic acid coding sequence of a microbial Argonaute protein are replaced with a UGG codon. In some embodiments, at least 70%, at least 80%, at least 90%, at least 95%, at least 99%, or 100%, of the ACG codons in the nucleic acid coding sequence of a microbial Argonaute protein are replaced with a CUU codon. In some embodiments, at least 70%, at least 80%, at least 90%, at least 95%, at least 99%, or 100%, of the ACG codons in the nucleic acid coding sequence of a microbial Argonaute protein are replaced with a CCU codon. In some embodiments, at least 70%, at least 80%, at least 90%, at least 95%, at least 99%, or 100%, of the ACG codons in the nucleic acid coding sequence of a microbial Argonaute protein are replaced with a CAU codon. In some embodiments, at least 70%, at least 80%, at least 90%, at least 95%, at least 99%, or 100%, of the ACG codons in the nucleic acid coding sequence of a microbial Argonaute protein are replaced with a CGU codon. In some embodiments, at least 70%, at least 80%, at least 90%, at least 95%, at least 99%, or 100%, of the ACG codons in the nucleic acid coding sequence of a microbial Argonaute protein are replaced with a CUC codon. In some embodiments, at least 70%, at least 80%, at least 90%, at least 95%, at least 99%, or 100%, of the ACG codons in the nucleic acid coding sequence of a microbial Argonaute protein are replaced with a CCC codon. In some embodiments, at least 70%, at least 80%, at least 90%, at least 95%, at least 99%, or 100%, of the ACG codons in the nucleic acid coding sequence of a microbial Argonaute protein are replaced with a CAC codon. In some embodiments, at least 70%, at least 80%, at least 90%, at least 95%, at least 99%, or 100%, of the ACG codons in the nucleic acid coding sequence of a microbial Argonaute protein are replaced with a CGC codon. In some embodiments, at least 70%, at least 80%, at least 90%, at least 95%, at least 99%, or 100%, of the ACG codons in the nucleic acid coding sequence of a microbial Argonaute protein are replaced with a CUA codon. In some embodiments, at least 70%, at least 80%, at least 90%, at least 95%, at least 99%, or 100%, of the ACG codons in the nucleic acid coding sequence of a microbial Argonaute protein are replaced with a CCA codon. In some embodiments, at least 70%, at least 80%, at least 90%, at least 95%, at least 99%, or 100%, of the ACG codons in the nucleic acid coding sequence of a microbial Argonaute protein are replaced with a CAA codon. In some embodiments, at least 70%, at least 80%, at least 90%, at least 95%, at least 99%, or 100%, of the ACG codons in the nucleic acid coding sequence of a microbial Argonaute protein are replaced with a CGA codon. In some embodiments, at least 70%, at least 80%, at least 90%, at least 95%, at least 99%, or 100%, of the ACG codons in the nucleic acid coding sequence of a microbial Argonaute protein are replaced with a CUG codon. In some embodiments, at least 70%, at least 80%, at least 90%, at least 95%, at least 99%, or 100%, of the ACG codons in the nucleic acid coding sequence of a microbial Argonaute protein are replaced with a CCG codon. In some embodiments, at least 70%, at least 80%, at least 90%, at least 95%, at least 99%, or 100%, of the ACG codons in the nucleic acid coding sequence of a microbial Argonaute protein are replaced with a CAG codon. In some embodiments, at least 70%, at least 80%, at least 90%, at least 95%, at least 99%, or 100%, of the ACG codons in the nucleic acid coding sequence of a microbial Argonaute protein are replaced with a CGG codon. In some embodiments, at least 70%, at least 80%, at least 90%, at least 95%, at least 99%, or 100%, of the ACG codons in the nucleic acid coding sequence of a microbial Argonaute protein are replaced with an AUU codon. In some embodiments, at least 70%, at least 80%, at least 90%, at least 95%, at least 99%, or 100%, of the ACG codons in the nucleic acid coding sequence of a microbial Argonaute protein are replaced with a ACU codon. In some embodiments, at least 70%, at least 80%, at least 90%, at least 95%, at least 99%, or 100%, of the ACG codons in the nucleic acid coding sequence of a microbial Argonaute protein are replaced with an AAU codon. In some embodiments, at least 70%, at least 80%, at least 90%, at least 95%, at least 99%, or 100%, of the ACG codons in the nucleic acid coding sequence of a microbial Argonaute protein are replaced with a AGU codon. In some embodiments, at least 70%, at least 80%, at least 90%, at least 95%, at least 99%, or 100%, of the ACG codons in the nucleic acid coding sequence of a microbial Argonaute protein are replaced with a AUC codon. In some embodiments, at least 70%, at least 80%, at least 90%, at least 95%, at least 99%, or 100%, of the ACG codons in the nucleic acid coding sequence of a microbial Argonaute protein are replaced with an ACC codon. In some embodiments, at least 70%, at least 80%, at least 90%, at least 95%, at least 99%, or 100%, of the ACG codons in the nucleic acid coding sequence of a microbial Argonaute protein are replaced with a AAC codon. In some embodiments, at least 70%, at least 80%, at least 90%, at least 95%, at least 99%, or 100%, of the ACG codons in the nucleic acid coding sequence of a microbial Argonaute protein are replaced with a AGC codon. In some embodiments, at least 70%, at least 80%, at least 90%, at least 95%, at least 99%, or 100%, of the ACG codons in the nucleic acid coding sequence of a microbial Argonaute protein are replaced with an AUA codon. In some embodiments, at least 70%, at least 80%, at least 90%, at least 95%, at least 99%, or 100%, of the ACG codons in the nucleic acid coding sequence of a microbial Argonaute protein are replaced with an ACA codon. In some embodiments, at least 70%, at least 80%, at least 90%, at least 95%, at least 99%, or 100%, of the ACG codons in the nucleic acid coding sequence of a microbial Argonaute protein are replaced with a AAA codon. In some embodiments, at least 70%, at least 80%, at least 90%, at least 95%, at least 99%, or 100%, of the ACG codons in the nucleic acid coding sequence of a microbial Argonaute protein are replaced with an AGA codon. In some embodiments, at least 70%, at least 80%, at least 90%, at least 95%, at least 99%, or 100%, of the ACG codons in the nucleic acid coding sequence of a microbial Argonaute protein are replaced with an AUG codon. In some embodiments, at least 70%, at least 80%, at least 90%, at least 95%, at least 99%, or 100%, of the ACG codons in the nucleic acid coding sequence of a microbial Argonaute protein are replaced with an ACG codon. In some embodiments, at least 70%, at least 80%, at least 90%, at least 95%, at least 99%, or 100%, of the ACG codons in the nucleic acid coding sequence of a microbial Argonaute protein are replaced with an AAG codon. In some embodiments, at least 70%, at least 80%, at least 90%, at least 95%, at least 99%, or 100%, of the ACG codons in the nucleic acid coding sequence of a microbial Argonaute protein are replaced with an AGG codon. In some embodiments, at least 70%, at least 80%, at least 90%, at least 95%, at least 99%, or 100%, of the ACG codons in the nucleic acid coding sequence of a microbial Argonaute protein are replaced with a GUU codon. In some embodiments, at least 70%, at least 80%, at least 90%, at least 95%, at least 99%, or 100%, of the ACG codons in the nucleic acid coding sequence of a microbial Argonaute protein are replaced with a GCU codon. In some embodiments, at least 70%, at least 80%, at least 90%, at least 95%, at least 99%, or 100%, of the ACG codons in the nucleic acid coding sequence of a microbial Argonaute protein are replaced with a GAU codon. In some embodiments, at least 70%, at least 80%, at least 90%, at least 95%, at least 99%, or 100%, of the ACG codons in the nucleic acid coding sequence of a microbial Argonaute protein are replaced with a GGU codon. In some embodiments, at least 70%, at least 80%, at least 90%, at least 95%, at least 99%, or 100%, of the ACG codons in the nucleic acid coding sequence of a microbial Argonaute protein are replaced with a GUC codon. In some embodiments, at least 70%, at least 80%, at least 90%, at least 95%, at least 99%, or 100%, of the ACG codons in the nucleic acid coding sequence of a microbial Argonaute protein are replaced with a GCC codon. In some embodiments, at least 70%, at least 80%, at least 90%, at least 95%, at least 99%, or 100%, of the ACG codons in the nucleic acid coding sequence of a microbial Argonaute protein are replaced with a GAC codon. In some embodiments, at least 70%, at least 80%, at least 90%, at least 95%, at least 99%, or 100%, of the ACG codons in the nucleic acid coding sequence of a microbial Argonaute protein are replaced with a GGC codon. In some embodiments, at least 70%, at least 80%, at least 90%, at least 95%, at least 99%, or 100%, of the ACG codons in the nucleic acid coding sequence of a microbial Argonaute protein are replaced with a GUA codon. In some embodiments, at least 70%, at least 80%, at least 90%, at least 95%, at least 99%, or 100%, of the ACG codons in the nucleic acid coding sequence of a microbial Argonaute protein are replaced with a GCA codon. In some embodiments, at least 70%, at least 80%, at least 90%, at least 95%, at least 99%, or 100%, of the ACG codons in the nucleic acid coding sequence of a microbial Argonaute protein are replaced with a GAA codon. In some embodiments, at least 70%, at least 80%, at least 90%, at least 95%, at least 99%, or 100%, of the ACG codons in the nucleic acid coding sequence of a microbial Argonaute protein are replaced with a GGA codon. In some embodiments, at least 70%, at least 80%, at least 90%, at least 95%, at least 99%, or 100%, of the ACG codons in the nucleic acid coding sequence of a microbial Argonaute protein are replaced with a GUG codon. In some embodiments, at least 70%, at least 80%, at least 90%, at least 95%, at least 99%, or 100%, of the ACG codons in the nucleic acid coding sequence of a microbial Argonaute protein are replaced with a GCG codon. In some embodiments, at least 70%, at least 80%, at least 90%, at least 95%, at least 99%, or 100%, of the ACG codons in the nucleic acid coding sequence of a microbial Argonaute protein are replaced with a GAG codon. In some embodiments, at least 70%, at least 80%, at least 90%, at least 95%, at least 99%, or 100%, of the ACG codons in the nucleic acid coding sequence of a microbial Argonaute protein are replaced with a GGG codon.

In some embodiments, at least 70%, at least 80%, at least 90%, at least 95%, at least 99%, or 100%, of the CCG codons in the nucleic acid coding sequence of a microbial Argonaute protein are replaced with a human preferred codon. In some embodiments, at least 70%, at least 80%, at least 90%, at least 95%, at least 99%, or 100%, of the CCG codons in the nucleic acid coding sequence of a microbial Argonaute protein are replaced with a UUU codon. In some embodiments, at least 70%, at least 80%, at least 90%, at least 95%, at least 99%, or 100%, of the CCG codons in the nucleic acid coding sequence of a microbial Argonaute protein are replaced with a UCU codon. In some embodiments, at least 70%, at least 80%, at least 90%, at least 95%, at least 99%, or 100%, of the CCG codons in the nucleic acid coding sequence of a microbial Argonaute protein are replaced with a UAU codon. In some embodiments, at least 70%, at least 80%, at least 90%, at least 95%, at least 99%, or 100%, of the CCG codons in the nucleic acid coding sequence of a microbial Argonaute protein are replaced with a UGU codon. In some embodiments, at least 70%, at least 80%, at least 90%, at least 95%, at least 99%, or 100%, of the CCG codons in the nucleic acid coding sequence of a microbial Argonaute protein are replaced with a UUC codon. In some embodiments, at least 70%, at least 80%, at least 90%, at least 95%, at least 99%, or 100%, of the CCG codons in the nucleic acid coding sequence of a microbial Argonaute protein are replaced with a UCC codon. In some embodiments, at least 70%, at least 80%, at least 90%, at least 95%, at least 99%, or 100%, of the CCG codons in the nucleic acid coding sequence of a microbial Argonaute protein are replaced with a UAC codon. In some embodiments, at least 70%, at least 80%, at least 90%, at least 95%, at least 99%, or 100%, of the CCG codons in the nucleic acid coding sequence of a microbial Argonaute protein are replaced with a UGC codon. In some embodiments, at least 70%, at least 80%, at least 90%, at least 95%, at least 99%, or 100%, of the CCG codons in the nucleic acid coding sequence of a microbial Argonaute protein are replaced with a UUA codon. In some embodiments, at least 70%, at least 80%, at least 90%, at least 95%, at least 99%, or 100%, of the CCG codons in the nucleic acid coding sequence of a microbial Argonaute protein are replaced with a UCA codon. In some embodiments, at least 70%, at least 80%, at least 90%, at least 95%, at least 99%, or 100%, of the CCG codons in the nucleic acid coding sequence of a microbial Argonaute protein are replaced with a UUG codon. In some embodiments, at least 70%, at least 80%, at least 90%, at least 95%, at least 99%, or 100%, of the CCG codons in the nucleic acid coding sequence of a microbial Argonaute protein are replaced with a UCG codon. In some embodiments, at least 70%, at least 80%, at least 90%, at least 95%, at least 99%, or 100%, of the CCG codons in the nucleic acid coding sequence of a microbial Argonaute protein are replaced with a UGG codon. In some embodiments, at least 70%, at least 80%, at least 90%, at least 95%, at least 99%, or 100%, of the CCG codons in the nucleic acid coding sequence of a microbial Argonaute protein are replaced with a CUU codon. In some embodiments, at least 70%, at least 80%, at least 90%, at least 95%, at least 99%, or 100%, of the CCG codons in the nucleic acid coding sequence of a microbial Argonaute protein are replaced with a CCU codon. In some embodiments, at least 70%, at least 80%, at least 90%, at least 95%, at least 99%, or 100%, of the CCG codons in the nucleic acid coding sequence of a microbial Argonaute protein are replaced with a CAU codon. In some embodiments, at least 70%, at least 80%, at least 90%, at least 95%, at least 99%, or 100%, of the CCG codons in the nucleic acid coding sequence of a microbial Argonaute protein are replaced with a CGU codon. In some embodiments, at least 70%, at least 80%, at least 90%, at least 95%, at least 99%, or 100%, of the CCG codons in the nucleic acid coding sequence of a microbial Argonaute protein are replaced with a CUC codon. In some embodiments, at least 70%, at least 80%, at least 90%, at least 95%, at least 99%, or 100%, of the CCG codons in the nucleic acid coding sequence of a microbial Argonaute protein are replaced with a CCC codon. In some embodiments, at least 70%, at least 80%, at least 90%, at least 95%, at least 99%, or 100%, of the CCG codons in the nucleic acid coding sequence of a microbial Argonaute protein are replaced with a CAC codon. In some embodiments, at least 70%, at least 80%, at least 90%, at least 95%, at least 99%, or 100%, of the CCG codons in the nucleic acid coding sequence of a microbial Argonaute protein are replaced with a CGC codon. In some embodiments, at least 70%, at least 80%, at least 90%, at least 95%, at least 99%, or 100%, of the CCG codons in the nucleic acid coding sequence of a microbial Argonaute protein are replaced with a CUA codon. In some embodiments, at least 70%, at least 80%, at least 90%, at least 95%, at least 99%, or 100%, of the CCG codons in the nucleic acid coding sequence of a microbial Argonaute protein are replaced with a CCA codon. In some embodiments, at least 70%, at least 80%, at least 90%, at least 95%, at least 99%, or 100%, of the CCG codons in the nucleic acid coding sequence of a microbial Argonaute protein are replaced with a CAA codon. In some embodiments, at least 70%, at least 80%, at least 90%, at least 95%, at least 99%, or 100%, of the CCG codons in the nucleic acid coding sequence of a microbial Argonaute protein are replaced with a CGA codon. In some embodiments, at least 70%, at least 80%, at least 90%, at least 95%, at least 99%, or 100%, of the CCG codons in the nucleic acid coding sequence of a microbial Argonaute protein are replaced with a CUG codon. In some embodiments, at least 70%, at least 80%, at least 90%, at least 95%, at least 99%, or 100%, of the CCG codons in the nucleic acid coding sequence of a microbial Argonaute protein are replaced with a CCG codon. In some embodiments, at least 70%, at least 80%, at least 90%, at least 95%, at least 99%, or 100%, of the CCG codons in the nucleic acid coding sequence of a microbial Argonaute protein are replaced with a CAG codon. In some embodiments, at least 70%, at least 80%, at least 90%, at least 95%, at least 99%, or 100%, of the CCG codons in the nucleic acid coding sequence of a microbial Argonaute protein are replaced with a CGG codon. In some embodiments, at least 70%, at least 80%, at least 90%, at least 95%, at least 99%, or 100%, of the CCG codons in the nucleic acid coding sequence of a microbial Argonaute protein are replaced with an AUU codon. In some embodiments, at least 70%, at least 80%, at least 90%, at least 95%, at least 99%, or 100%, of the CCG codons in the nucleic acid coding sequence of a microbial Argonaute protein are replaced with an ACU codon. In some embodiments, at least 70%, at least 80%, at least 90%, at least 95%, at least 99%, or 100%, of the CCG codons in the nucleic acid coding sequence of a microbial Argonaute protein are replaced with an AAU codon. In some embodiments, at least 70%, at least 80%, at least 90%, at least 95%, at least 99%, or 100%, of the CCG codons in the nucleic acid coding sequence of a microbial Argonaute protein are replaced with an AGU codon. In some embodiments, at least 70%, at least 80%, at least 90%, at least 95%, at least 99%, or 100%, of the CCG codons in the nucleic acid coding sequence of a microbial Argonaute protein are replaced with an AUC codon. In some embodiments, at least 70%, at least 80%, at least 90%, at least 95%, at least 99%, or 100%, of the CCG codons in the nucleic acid coding sequence of a microbial Argonaute protein are replaced with an ACC codon. In some embodiments, at least 70%, at least 80%, at least 90%, at least 95%, at least 99%, or 100%, of the CCG codons in the nucleic acid coding sequence of a microbial Argonaute protein are replaced with an AAC codon. In some embodiments, at least 70%, at least 80%, at least 90%, at least 95%, at least 99%, or 100%, of the CCG codons in the nucleic acid coding sequence of a microbial Argonaute protein are replaced with an AGC codon. In some embodiments, at least 70%, at least 80%, at least 90%, at least 95%, at least 99%, or 100%, of the CCG codons in the nucleic acid coding sequence of a microbial Argonaute protein are replaced with an AUA codon. In some embodiments, at least 70%, at least 80%, at least 90%, at least 95%, at least 99%, or 100%, of the CCG codons in the nucleic acid coding sequence of a microbial Argonaute protein are replaced with an ACA codon. In some embodiments, at least 70%, at least 80%, at least 90%, at least 95%, at least 99%, or 100%, of the CCG codons in the nucleic acid coding sequence of a microbial Argonaute protein are replaced with a AAA codon. In some embodiments, at least 70%, at least 80%, at least 90%, at least 95%, at least 99%, or 100%, of the CCG codons in the nucleic acid coding sequence of a microbial Argonaute protein are replaced with an AGA codon. In some embodiments, at least 70%, at least 80%, at least 90%, at least 95%, at least 99%, or 100%, of the CCG codons in the nucleic acid coding sequence of a microbial Argonaute protein are replaced with an AUG codon. In some embodiments, at least 70%, at least 80%, at least 90%, at least 95%, at least 99%, or 100%, of the CCG codons in the nucleic acid coding sequence of a microbial Argonaute protein are replaced with an ACG codon. In some embodiments, at least 70%, at least 80%, at least 90%, at least 95%, at least 99%, or 100%, of the CCG codons in the nucleic acid coding sequence of a microbial Argonaute protein are replaced with an AAG codon. In some embodiments, at least 70%, at least 80%, at least 90%, at least 95%, at least 99%, or 100%, of the CCG codons in the nucleic acid coding sequence of a microbial Argonaute protein are replaced with an AGG codon. In some embodiments, at least 70%, at least 80%, at least 90%, at least 95%, at least 99%, or 100%, of the CCG codons in the nucleic acid coding sequence of a microbial Argonaute protein are replaced with a GUU codon. In some embodiments, at least 70%, at least 80%, at least 90%, at least 95%, at least 99%, or 100%, of the CCG codons in the nucleic acid coding sequence of a microbial Argonaute protein are replaced with a GCU codon. In some embodiments, at least 70%, at least 80%, at least 90%, at least 95%, at least 99%, or 100%, of the CCG codons in the nucleic acid coding sequence of a microbial Argonaute protein are replaced with a GAU codon. In some embodiments, at least 70%, at least 80%, at least 90%, at least 95%, at least 99%, or 100%, of the CCG codons in the nucleic acid coding sequence of a microbial Argonaute protein are replaced with a GGU codon. In some embodiments, at least 70%, at least 80%, at least 90%, at least 95%, at least 99%, or 100%, of the CCG codons in the nucleic acid coding sequence of a microbial Argonaute protein are replaced with a GUC codon. In some embodiments, at least 70%, at least 80%, at least 90%, at least 95%, at least 99%, or 100%, of the CCG codons in the nucleic acid coding sequence of a microbial Argonaute protein are replaced with a GCC codon. In some embodiments, at least 70%, at least 80%, at least 90%, at least 95%, at least 99%, or 100%, of the CCG codons in the nucleic acid coding sequence of a microbial Argonaute protein are replaced with a GAC codon. In some embodiments, at least 70%, at least 80%, at least 90%, at least 95%, at least 99%, or 100%, of the CCG codons in the nucleic acid coding sequence of a microbial Argonaute protein are replaced with a GGC codon. In some embodiments, at least 70%, at least 80%, at least 90%, at least 95%, at least 99%, or 100%, of the CCG codons in the nucleic acid coding sequence of a microbial Argonaute protein are replaced with a GUA codon. In some embodiments, at least 70%, at least 80%, at least 90%, at least 95%, at least 99%, or 100%, of the CCG codons in the nucleic acid coding sequence of a microbial Argonaute protein are replaced with a GCA codon. In some embodiments, at least 70%, at least 80%, at least 90%, at least 95%, at least 99%, or 100%, of the CCG codons in the nucleic acid coding sequence of a microbial Argonaute protein are replaced with a GAA codon. In some embodiments, at least 70%, at least 80%, at least 90%, at least 95%, at least 99%, or 100%, of the CCG codons in the nucleic acid coding sequence of a microbial Argonaute protein are replaced with a GGA codon. In some embodiments, at least 70%, at least 80%, at least 90%, at least 95%, at least 99%, or 100%, of the CCG codons in the nucleic acid coding sequence of a microbial Argonaute protein are replaced with a GUG codon. In some embodiments, at least 70%, at least 80%, at least 90%, at least 95%, at least 99%, or 100%, of the CCG codons in the nucleic acid coding sequence of a microbial Argonaute protein are replaced with a GCG codon. In some embodiments, at least 70%, at least 80%, at least 90%, at least 95%, at least 99%, or 100%, of the CCG codons in the nucleic acid coding sequence of a microbial Argonaute protein are replaced with a GAG codon. In some embodiments, at least 70%, at least 80%, at least 90%, at least 95%, at least 99%, or 100%, of the CCG codons in the nucleic acid coding sequence of a microbial Argonaute protein are replaced with a GGG codon.

In some embodiments, at least 70%, at least 80%, at least 90%, at least 95%, at least 99%, or 100%, of the TCG codons in the nucleic acid coding sequence of a microbial Argonaute protein are replaced with a human preferred codon. In some embodiments, at least 70%, at least 80%, at least 90%, at least 95%, at least 99%, or 100%, of the TCG codons in the nucleic acid coding sequence of a microbial Argonaute protein are replaced with a UUU codon. In some embodiments, at least 70%, at least 80%, at least 90%, at least 95%, at least 99%, or 100%, of the TCG codons in the nucleic acid coding sequence of a microbial Argonaute protein are replaced with a UCU codon. In some embodiments, at least 70%, at least 80%, at least 90%, at least 95%, at least 99%, or 100%, of the TCG codons in the nucleic acid coding sequence of a microbial Argonaute protein are replaced with a UAU codon. In some embodiments, at least 70%, at least 80%, at least 90%, at least 95%, at least 99%, or 100%, of the TCG codons in the nucleic acid coding sequence of a microbial Argonaute protein are replaced with a UGU codon. In some embodiments, at least 70%, at least 80%, at least 90%, at least 95%, at least 99%, or 100%, of the TCG codons in the nucleic acid coding sequence of a microbial Argonaute protein are replaced with a UUC codon. In some embodiments, at least 70%, at least 80%, at least 90%, at least 95%, at least 99%, or 100%, of the TCG codons in the nucleic acid coding sequence of a microbial Argonaute protein are replaced with a UCC codon. In some embodiments, at least 70%, at least 80%, at least 90%, at least 95%, at least 99%, or 100%, of the TCG codons in the nucleic acid coding sequence of a microbial Argonaute protein are replaced with a UAC codon. In some embodiments, at least 70%, at least 80%, at least 90%, at least 95%, at least 99%, or 100%, of the TCG codons in the nucleic acid coding sequence of a microbial Argonaute protein are replaced with a UGC codon. In some embodiments, at least 70%, at least 80%, at least 90%, at least 95%, at least 99%, or 100%, of the TCG codons in the nucleic acid coding sequence of a microbial Argonaute protein are replaced with a UUA codon. In some embodiments, at least 70%, at least 80%, at least 90%, at least 95%, at least 99%, or 100%, of the TCG codons in the nucleic acid coding sequence of a microbial Argonaute protein are replaced with a UCA codon. In some embodiments, at least 70%, at least 80%, at least 90%, at least 95%, at least 99%, or 100%, of the TCG codons in the nucleic acid coding sequence of a microbial Argonaute protein are replaced with a UUG codon. In some embodiments, at least 70%, at least 80%, at least 90%, at least 95%, at least 99%, or 100%, of the TCG codons in the nucleic acid coding sequence of a microbial Argonaute protein are replaced with a UCG codon. In some embodiments, at least 70%, at least 80%, at least 90%, at least 95%, at least 99%, or 100%, of the TCG codons in the nucleic acid coding sequence of a microbial Argonaute protein are replaced with a UGG codon. In some embodiments, at least 70%, at least 80%, at least 90%, at least 95%, at least 99%, or 100%, of the TCG codons in the nucleic acid coding sequence of a microbial Argonaute protein are replaced with a CUU codon. In some embodiments, at least 70%, at least 80%, at least 90%, at least 95%, at least 99%, or 100%, of the TCG codons in the nucleic acid coding sequence of a microbial Argonaute protein are replaced with a CCU codon. In some embodiments, at least 70%, at least 80%, at least 90%, at least 95%, at least 99%, or 100%, of the TCG codons in the nucleic acid coding sequence of a microbial Argonaute protein are replaced with a CAU codon. In some embodiments, at least 70%, at least 80%, at least 90%, at least 95%, at least 99%, or 100%, of the TCG codons in the nucleic acid coding sequence of a microbial Argonaute protein are replaced with a CGU codon. In some embodiments, at least 70%, at least 80%, at least 90%, at least 95%, at least 99%, or 100%, of the TCG codons in the nucleic acid coding sequence of a microbial Argonaute protein are replaced with a CUC codon. In some embodiments, at least 70%, at least 80%, at least 90%, at least 95%, at least 99%, or 100%, of the TCG codons in the nucleic acid coding sequence of a microbial Argonaute protein are replaced with a CCC codon. In some embodiments, at least 70%, at least 80%, at least 90%, at least 95%, at least 99%, or 100%, of the TCG codons in the nucleic acid coding sequence of a microbial Argonaute protein are replaced with a CAC codon. In some embodiments, at least 70%, at least 80%, at least 90%, at least 95%, at least 99%, or 100%, of the TCG codons in the nucleic acid coding sequence of a microbial Argonaute protein are replaced with a CGC codon. In some embodiments, at least 70%, at least 80%, at least 90%, at least 95%, at least 99%, or 100%, of the TCG codons in the nucleic acid coding sequence of a microbial Argonaute protein are replaced with a CUA codon. In some embodiments, at least 70%, at least 80%, at least 90%, at least 95%, at least 99%, or 100%, of the TCG codons in the nucleic acid coding sequence of a microbial Argonaute protein are replaced with a CCA codon. In some embodiments, at least 70%, at least 80%, at least 90%, at least 95%, at least 99%, or 100%, of the TCG codons in the nucleic acid coding sequence of a microbial Argonaute protein are replaced with a CAA codon. In some embodiments, at least 70%, at least 80%, at least 90%, at least 95%, at least 99%, or 100%, of the TCG codons in the nucleic acid coding sequence of a microbial Argonaute protein are replaced with a CGA codon. In some embodiments, at least 70%, at least 80%, at least 90%, at least 95%, at least 99%, or 100%, of the TCG codons in the nucleic acid coding sequence of a microbial Argonaute protein are replaced with a CUG codon. In some embodiments, at least 70%, at least 80%, at least 90%, at least 95%, at least 99%, or 100%, of the TCG codons in the nucleic acid coding sequence of a microbial Argonaute protein are replaced with a CCG codon. In some embodiments, at least 70%, at least 80%, at least 90%, at least 95%, at least 99%, or 100%, of the TCG codons in the nucleic acid coding sequence of a microbial Argonaute protein are replaced with a CAG codon. In some embodiments, at least 70%, at least 80%, at least 90%, at least 95%, at least 99%, or 100%, of the TCG codons in the nucleic acid coding sequence of a microbial Argonaute protein are replaced with a CGG codon. In some embodiments, at least 70%, at least 80%, at least 90%, at least 95%, at least 99%, or 100%, of the TCG codons in the nucleic acid coding sequence of a microbial Argonaute protein are replaced with an AUU codon. In some embodiments, at least 70%, at least 80%, at least 90%, at least 95%, at least 99%, or 100%, of the TCG codons in the nucleic acid coding sequence of a microbial Argonaute protein are replaced with an ACU codon. In some embodiments, at least 70%, at least 80%, at least 90%, at least 95%, at least 99%, or 100%, of the TCG codons in the nucleic acid coding sequence of a microbial Argonaute protein are replaced with an AAU codon. In some embodiments, at least 70%, at least 80%, at least 90%, at least 95%, at least 99%, or 100%, of the TCG codons in the nucleic acid coding sequence of a microbial Argonaute protein are replaced with an AGU codon. In some embodiments, at least 70%, at least 80%, at least 90%, at least 95%, at least 99%, or 100%, of the TCG codons in the nucleic acid coding sequence of a microbial Argonaute protein are replaced with an AUC codon. In some embodiments, at least 70%, at least 80%, at least 90%, at least 95%, at least 99%, or 100%, of the TCG codons in the nucleic acid coding sequence of a microbial Argonaute protein are replaced with an ACC codon. In some embodiments, at least 70%, at least 80%, at least 90%, at least 95%, at least 99%, or 100%, of the TCG codons in the nucleic acid coding sequence of a microbial Argonaute protein are replaced with an AAC codon. In some embodiments, at least 70%, at least 80%, at least 90%, at least 95%, at least 99%, or 100%, of the TCG codons in the nucleic acid coding sequence of a microbial Argonaute protein are replaced with an AGC codon. In some embodiments, at least 70%, at least 80%, at least 90%, at least 95%, at least 99%, or 100%, of the TCG codons in the nucleic acid coding sequence of a microbial Argonaute protein are replaced with an AUA codon. In some embodiments, at least 70%, at least 80%, at least 90%, at least 95%, at least 99%, or 100%, of the TCG codons in the nucleic acid coding sequence of a microbial Argonaute protein are replaced with an ACA codon. In some embodiments, at least 70%, at least 80%, at least 90%, at least 95%, at least 99%, or 100%, of the TCG codons in the nucleic acid coding sequence of a microbial Argonaute protein are replaced with a AAA codon. In some embodiments, at least 70%, at least 80%, at least 90%, at least 95%, at least 99%, or 100%, of the TCG codons in the nucleic acid coding sequence of a microbial Argonaute protein are replaced with an AGA codon. In some embodiments, at least 70%, at least 80%, at least 90%, at least 95%, at least 99%, or 100%, of the TCG codons in the nucleic acid coding sequence of a microbial Argonaute protein are replaced with an AUG codon. In some embodiments, at least 70%, at least 80%, at least 90%, at least 95%, at least 99%, or 100%, of the TCG codons in the nucleic acid coding sequence of a microbial Argonaute protein are replaced with an ACG codon. In some embodiments, at least 70%, at least 80%, at least 90%, at least 95%, at least 99%, or 100%, of the TCG codons in the nucleic acid coding sequence of a microbial Argonaute protein are replaced with an AAG codon. In some embodiments, at least 70%, at least 80%, at least 90%, at least 95%, at least 99%, or 100%, of the TCG codons in the nucleic acid coding sequence of a microbial Argonaute protein are replaced with an AGG codon. In some embodiments, at least 70%, at least 80%, at least 90%, at least 95%, at least 99%, or 100%, of the TCG codons in the nucleic acid coding sequence of a microbial Argonaute protein are replaced with a GUU codon. In some embodiments, at least 70%, at least 80%, at least 90%, at least 95%, at least 99%, or 100%, of the TCG codons in the nucleic acid coding sequence of a microbial Argonaute protein are replaced with a GCU codon. In some embodiments, at least 70%, at least 80%, at least 90%, at least 95%, at least 99%, or 100%, of the TCG codons in the nucleic acid coding sequence of a microbial Argonaute protein are replaced with a GAU codon. In some embodiments, at least 70%, at least 80%, at least 90%, at least 95%, at least 99%, or 100%, of the TCG codons in the nucleic acid coding sequence of a microbial Argonaute protein are replaced with a GGU codon. In some embodiments, at least 70%, at least 80%, at least 90%, at least 95%, at least 99%, or 100%, of the TCG codons in the nucleic acid coding sequence of a microbial Argonaute protein are replaced with a GUC codon. In some embodiments, at least 70%, at least 80%, at least 90%, at least 95%, at least 99%, or 100%, of the TCG codons in the nucleic acid coding sequence of a microbial Argonaute protein are replaced with a GCC codon. In some embodiments, at least 70%, at least 80%, at least 90%, at least 95%, at least 99%, or 100%, of the TCG codons in the nucleic acid coding sequence of a microbial Argonaute protein are replaced with a GAC codon. In some embodiments, at least 70%, at least 80%, at least 90%, at least 95%, at least 99%, or 100%, of the TCG codons in the nucleic acid coding sequence of a microbial Argonaute protein are replaced with a GGC codon. In some embodiments, at least 70%, at least 80%, at least 90%, at least 95%, at least 99%, or 100%, of the TCG codons in the nucleic acid coding sequence of a microbial Argonaute protein are replaced with a GUA codon. In some embodiments, at least 70%, at least 80%, at least 90%, at least 95%, at least 99%, or 100%, of the TCG codons in the nucleic acid coding sequence of a microbial Argonaute protein are replaced with a GCA codon. In some embodiments, at least 70%, at least 80%, at least 90%, at least 95%, at least 99%, or 100%, of the TCG codons in the nucleic acid coding sequence of a microbial Argonaute protein are replaced with a GAA codon. In some embodiments, at least 70%, at least 80%, at least 90%, at least 95%, at least 99%, or 100%, of the TCG codons in the nucleic acid coding sequence of a microbial Argonaute protein are replaced with a GGA codon. In some embodiments, at least 70%, at least 80%, at least 90%, at least 95%, at least 99%, or 100%, of the TCG codons in the nucleic acid coding sequence of a microbial Argonaute protein are replaced with a GUG codon. In some embodiments, at least 70%, at least 80%, at least 90%, at least 95%, at least 99%, or 100%, of the TCG codons in the nucleic acid coding sequence of a microbial Argonaute protein are replaced with a GCG codon. In some embodiments, at least 70%, at least 80%, at least 90%, at least 95%, at least 99%, or 100%, of the TCG codons in the nucleic acid coding sequence of a microbial Argonaute protein are replaced with a GAG codon. In some embodiments, at least 70%, at least 80%, at least 90%, at least 95%, at least 99%, or 100%, of the TCG codons in the nucleic acid coding sequence of a microbial Argonaute protein are replaced with a GGG codon.

In some embodiments, at least 70%, at least 80%, at least 90%, at least 95%, at least 99%, or 100%, of the GTA codons in the nucleic acid coding sequence of a microbial Argonaute protein are replaced with a human preferred codon. In some embodiments, at least 70%, at least 80%, at least 90%, at least 95%, at least 99%, or 100%, of the GTA codons in the nucleic acid coding sequence of a microbial Argonaute protein are replaced with a UUU codon. In some embodiments, at least 70%, at least 80%, at least 90%, at least 95%, at least 99%, or 100%, of the GTA codons in the nucleic acid coding sequence of a microbial Argonaute protein are replaced with a UCU codon. In some embodiments, at least 70%, at least 80%, at least 90%, at least 95%, at least 99%, or 100%, of the GTA codons in the nucleic acid coding sequence of a microbial Argonaute protein are replaced with a UAU codon. In some embodiments, at least 70%, at least 80%, at least 90%, at least 95%, at least 99%, or 100%, of the GTA codons in the nucleic acid coding sequence of a microbial Argonaute protein are replaced with a UGU codon. In some embodiments, at least 70%, at least 80%, at least 90%, at least 95%, at least 99%, or 100%, of the GTA codons in the nucleic acid coding sequence of a microbial Argonaute protein are replaced with a UUC codon. In some embodiments, at least 70%, at least 80%, at least 90%, at least 95%, at least 99%, or 100%, of the GTA codons in the nucleic acid coding sequence of a microbial Argonaute protein are replaced with a UCC codon. In some embodiments, at least 70%, at least 80%, at least 90%, at least 95%, at least 99%, or 100%, of the GTA codons in the nucleic acid coding sequence of a microbial Argonaute protein are replaced with a UAC codon. In some embodiments, at least 70%, at least 80%, at least 90%, at least 95%, at least 99%, or 100%, of the GTA codons in the nucleic acid coding sequence of a microbial Argonaute protein are replaced with a UGC codon. In some embodiments, at least 70%, at least 80%, at least 90%, at least 95%, at least 99%, or 100%, of the GTA codons in the nucleic acid coding sequence of a microbial Argonaute protein are replaced with a UUA codon. In some embodiments, at least 70%, at least 80%, at least 90%, at least 95%, at least 99%, or 100%, of the GTA codons in the nucleic acid coding sequence of a microbial Argonaute protein are replaced with a UCA codon. In some embodiments, at least 70%, at least 80%, at least 90%, at least 95%, at least 99%, or 100%, of the GTA codons in the nucleic acid coding sequence of a microbial Argonaute protein are replaced with a UUG codon. In some embodiments, at least 70%, at least 80%, at least 90%, at least 95%, at least 99%, or 100%, of the GTA codons in the nucleic acid coding sequence of a microbial Argonaute protein are replaced with a UCG codon. In some embodiments, at least 70%, at least 80%, at least 90%, at least 95%, at least 99%, or 100%, of the GTA codons in the nucleic acid coding sequence of a microbial Argonaute protein are replaced with a UGG codon. In some embodiments, at least 70%, at least 80%, at least 90%, at least 95%, at least 99%, or 100%, of the GTA codons in the nucleic acid coding sequence of a microbial Argonaute protein are replaced with a CUU codon. In some embodiments, at least 70%, at least 80%, at least 90%, at least 95%, at least 99%, or 100%, of the GTA codons in the nucleic acid coding sequence of a microbial Argonaute protein are replaced with a CCU codon. In some embodiments, at least 70%, at least 80%, at least 90%, at least 95%, at least 99%, or 100%, of the GTA codons in the nucleic acid coding sequence of a microbial Argonaute protein are replaced with a CAU codon. In some embodiments, at least 70%, at least 80%, at least 90%, at least 95%, at least 99%, or 100%, of the GTA codons in the nucleic acid coding sequence of a microbial Argonaute protein are replaced with a CGU codon. In some embodiments, at least 70%, at least 80%, at least 90%, at least 95%, at least 99%, or 100%, of the GTA codons in the nucleic acid coding sequence of a microbial Argonaute protein are replaced with a CUC codon. In some embodiments, at least 70%, at least 80%, at least 90%, at least 95%, at least 99%, or 100%, of the GTA codons in the nucleic acid coding sequence of a microbial Argonaute protein are replaced with a CCC codon. In some embodiments, at least 70%, at least 80%, at least 90%, at least 95%, at least 99%, or 100%, of the GTA codons in the nucleic acid coding sequence of a microbial Argonaute protein are replaced with a CAC codon. In some embodiments, at least 70%, at least 80%, at least 90%, at least 95%, at least 99%, or 100%, of the GTA codons in the nucleic acid coding sequence of a microbial Argonaute protein are replaced with a CGC codon. In some embodiments, at least 70%, at least 80%, at least 90%, at least 95%, at least 99%, or 100%, of the GTA codons in the nucleic acid coding sequence of a microbial Argonaute protein are replaced with a CUA codon. In some embodiments, at least 70%, at least 80%, at least 90%, at least 95%, at least 99%, or 100%, of the GTA codons in the nucleic acid coding sequence of a microbial Argonaute protein are replaced with a CCA codon. In some embodiments, at least 70%, at least 80%, at least 90%, at least 95%, at least 99%, or 100%, of the GTA codons in the nucleic acid coding sequence of a microbial Argonaute protein are replaced with a CAA codon. In some embodiments, at least 70%, at least 80%, at least 90%, at least 95%, at least 99%, or 100%, of the GTA codons in the nucleic acid coding sequence of a microbial Argonaute protein are replaced with a CGA codon. In some embodiments, at least 70%, at least 80%, at least 90%, at least 95%, at least 99%, or 100%, of the GTA codons in the nucleic acid coding sequence of a microbial Argonaute protein are replaced with a CUG codon. In some embodiments, at least 70%, at least 80%, at least 90%, at least 95%, at least 99%, or 100%, of the GTA codons in the nucleic acid coding sequence of a microbial Argonaute protein are replaced with a CCG codon. In some embodiments, at least 70%, at least 80%, at least 90%, at least 95%, at least 99%, or 100%, of the GTA codons in the nucleic acid coding sequence of a microbial Argonaute protein are replaced with a CAG codon. In some embodiments, at least 70%, at least 80%, at least 90%, at least 95%, at least 99%, or 100%, of the GTA codons in the nucleic acid coding sequence of a microbial Argonaute protein are replaced with a CGG codon. In some embodiments, at least 70%, at least 80%, at least 90%, at least 95%, at least 99%, or 100%, of the GTA codons in the nucleic acid coding sequence of a microbial Argonaute protein are replaced with an AUU codon. In some embodiments, at least 70%, at least 80%, at least 90%, at least 95%, at least 99%, or 100%, of the GTA codons in the nucleic acid coding sequence of a microbial Argonaute protein are replaced with an ACU codon. In some embodiments, at least 70%, at least 80%, at least 90%, at least 95%, at least 99%, or 100%, of the GTA codons in the nucleic acid coding sequence of a microbial Argonaute protein are replaced with an AAU codon. In some embodiments, at least 70%, at least 80%, at least 90%, at least 95%, at least 99%, or 100%, of the GTA codons in the nucleic acid coding sequence of a microbial Argonaute protein are replaced with an AGU codon. In some embodiments, at least 70%, at least 80%, at least 90%, at least 95%, at least 99%, or 100%, of the GTA codons in the nucleic acid coding sequence of a microbial Argonaute protein are replaced with an AUC codon. In some embodiments, at least 70%, at least 80%, at least 90%, at least 95%, at least 99%, or 100%, of the GTA codons in the nucleic acid coding sequence of a microbial Argonaute protein are replaced with an ACC codon. In some embodiments, at least 70%, at least 80%, at least 90%, at least 95%, at least 99%, or 100%, of the GTA codons in the nucleic acid coding sequence of a microbial Argonaute protein are replaced with an AAC codon. In some embodiments, at least 70%, at least 80%, at least 90%, at least 95%, at least 99%, or 100%, of the GTA codons in the nucleic acid coding sequence of a microbial Argonaute protein are replaced with an AGC codon. In some embodiments, at least 70%, at least 80%, at least 90%, at least 95%, at least 99%, or 100%, of the GTA codons in the nucleic acid coding sequence of a microbial Argonaute protein are replaced with an AUA codon. In some embodiments, at least 70%, at least 80%, at least 90%, at least 95%, at least 99%, or 100%, of the GTA codons in the nucleic acid coding sequence of a microbial Argonaute protein are replaced with an ACA codon. In some embodiments, at least 70%, at least 80%, at least 90%, at least 95%, at least 99%, or 100%, of the GTA codons in the nucleic acid coding sequence of a microbial Argonaute protein are replaced with a AAA codon. In some embodiments, at least 70%, at least 80%, at least 90%, at least 95%, at least 99%, or 100%, of the GTA codons in the nucleic acid coding sequence of a microbial Argonaute protein are replaced with an AGA codon. In some embodiments, at least 70%, at least 80%, at least 90%, at least 95%, at least 99%, or 100%, of the GTA codons in the nucleic acid coding sequence of a microbial Argonaute protein are replaced with an AUG codon. In some embodiments, at least 70%, at least 80%, at least 90%, at least 95%, at least 99%, or 100%, of the GTA codons in the nucleic acid coding sequence of a microbial Argonaute protein are replaced with an ACG codon. In some embodiments, at least 70%, at least 80%, at least 90%, at least 95%, at least 99%, or 100%, of the GTA codons in the nucleic acid coding sequence of a microbial Argonaute protein are replaced with an AAG codon. In some embodiments, at least 70%, at least 80%, at least 90%, at least 95%, at least 99%, or 100%, of the GTA codons in the nucleic acid coding sequence of a microbial Argonaute protein are replaced with an AGG codon. In some embodiments, at least 70%, at least 80%, at least 90%, at least 95%, at least 99%, or 100%, of the GTA codons in the nucleic acid coding sequence of a microbial Argonaute protein are replaced with a GUU codon. In some embodiments, at least 70%, at least 80%, at least 90%, at least 95%, at least 99%, or 100%, of the GTA codons in the nucleic acid coding sequence of a microbial Argonaute protein are replaced with a GCU codon. In some embodiments, at least 70%, at least 80%, at least 90%, at least 95%, at least 99%, or 100%, of the GTA codons in the nucleic acid coding sequence of a microbial Argonaute protein are replaced with a GAU codon. In some embodiments, at least 70%, at least 80%, at least 90%, at least 95%, at least 99%, or 100%, of the GTA codons in the nucleic acid coding sequence of a microbial Argonaute protein are replaced with a GGU codon. In some embodiments, at least 70%, at least 80%, at least 90%, at least 95%, at least 99%, or 100%, of the GTA codons in the nucleic acid coding sequence of a microbial Argonaute protein are replaced with a GUC codon. In some embodiments, at least 70%, at least 80%, at least 90%, at least 95%, at least 99%, or 100%, of the GTA codons in the nucleic acid coding sequence of a microbial Argonaute protein are replaced with a GCC codon. In some embodiments, at least 70%, at least 80%, at least 90%, at least 95%, at least 99%, or 100%, of the GTA codons in the nucleic acid coding sequence of a microbial Argonaute protein are replaced with a GAC codon. In some embodiments, at least 70%, at least 80%, at least 90%, at least 95%, at least 99%, or 100%, of the GTA codons in the nucleic acid coding sequence of a microbial Argonaute protein are replaced with a GGC codon. In some embodiments, at least 70%, at least 80%, at least 90%, at least 95%, at least 99%, or 100%, of the GTA codons in the nucleic acid coding sequence of a microbial Argonaute protein are replaced with a GUA codon. In some embodiments, at least 70%, at least 80%, at least 90%, at least 95%, at least 99%, or 100%, of the GTA codons in the nucleic acid coding sequence of a microbial Argonaute protein are replaced with a GCA codon. In some embodiments, at least 70%, at least 80%, at least 90%, at least 95%, at least 99%, or 100%, of the GTA codons in the nucleic acid coding sequence of a microbial Argonaute protein are replaced with a GAA codon. In some embodiments, at least 70%, at least 80%, at least 90%, at least 95%, at least 99%, or 100%, of the GTA codons in the nucleic acid coding sequence of a microbial Argonaute protein are replaced with a GGA codon. In some embodiments, at least 70%, at least 80%, at least 90%, at least 95%, at least 99%, or 100%, of the GTA codons in the nucleic acid coding sequence of a microbial Argonaute protein are replaced with a GUG codon. In some embodiments, at least 70%, at least 80%, at least 90%, at least 95%, at least 99%, or 100%, of the GTA codons in the nucleic acid coding sequence of a microbial Argonaute protein are replaced with a GCG codon. In some embodiments, at least 70%, at least 80%, at least 90%, at least 95%, at least 99%, or 100%, of the GTA codons in the nucleic acid coding sequence of a microbial Argonaute protein are replaced with a GAG codon. In some embodiments, at least 70%, at least 80%, at least 90%, at least 95%, at least 99%, or 100%, of the GTA codons in the nucleic acid coding sequence of a microbial Argonaute protein are replaced with a GGG codon.

In some embodiments, at least 70%, at least 80%, at least 90%, at least 95%, at least 99%, or 100%, of the CTA codons in the nucleic acid coding sequence of a microbial Argonaute protein are replaced with a human preferred codon. In some embodiments, at least 70%, at least 80%, at least 90%, at least 95%, at least 99%, or 100%, of the CTA codons in the nucleic acid coding sequence of a microbial Argonaute protein are replaced with a UUU codon. In some embodiments, at least 70%, at least 80%, at least 90%, at least 95%, at least 99%, or 100%, of the CTA codons in the nucleic acid coding sequence of a microbial Argonaute protein are replaced with a UCU codon. In some embodiments, at least 70%, at least 80%, at least 90%, at least 95%, at least 99%, or 100%, of the CTA codons in the nucleic acid coding sequence of a microbial Argonaute protein are replaced with a UAU codon. In some embodiments, at least 70%, at least 80%, at least 90%, at least 95%, at least 99%, or 100%, of the CTA codons in the nucleic acid coding sequence of a microbial Argonaute protein are replaced with a UGU codon. In some embodiments, at least 70%, at least 80%, at least 90%, at least 95%, at least 99%, or 100%, of the CTA codons in the nucleic acid coding sequence of a microbial Argonaute protein are replaced with a UUC codon. In some embodiments, at least 70%, at least 80%, at least 90%, at least 95%, at least 99%, or 100%, of the CTA codons in the nucleic acid coding sequence of a microbial Argonaute protein are replaced with a UCC codon. In some embodiments, at least 70%, at least 80%, at least 90%, at least 95%, at least 99%, or 100%, of the CTA codons in the nucleic acid coding sequence of a microbial Argonaute protein are replaced with a UAC codon. In some embodiments, at least 70%, at least 80%, at least 90%, at least 95%, at least 99%, or 100%, of the CTA codons in the nucleic acid coding sequence of a microbial Argonaute protein are replaced with a UGC codon. In some embodiments, at least 70%, at least 80%, at least 90%, at least 95%, at least 99%, or 100%, of the CTA codons in the nucleic acid coding sequence of a microbial Argonaute protein are replaced with a UUA codon. In some embodiments, at least 70%, at least 80%, at least 90%, at least 95%, at least 99%, or 100%, of the CTA codons in the nucleic acid coding sequence of a microbial Argonaute protein are replaced with a UCA codon. In some embodiments, at least 70%, at least 80%, at least 90%, at least 95%, at least 99%, or 100%, of the CTA codons in the nucleic acid coding sequence of a microbial Argonaute protein are replaced with a UUG codon. In some embodiments, at least 70%, at least 80%, at least 90%, at least 95%, at least 99%, or 100%, of the CTA codons in the nucleic acid coding sequence of a microbial Argonaute protein are replaced with a UCG codon. In some embodiments, at least 70%, at least 80%, at least 90%, at least 95%, at least 99%, or 100%, of the CTA codons in the nucleic acid coding sequence of a microbial Argonaute protein are replaced with a UGG codon. In some embodiments, at least 70%, at least 80%, at least 90%, at least 95%, at least 99%, or 100%, of the CTA codons in the nucleic acid coding sequence of a microbial Argonaute protein are replaced with a CUU codon. In some embodiments, at least 70%, at least 80%, at least 90%, at least 95%, at least 99%, or 100%, of the CTA codons in the nucleic acid coding sequence of a microbial Argonaute protein are replaced with a CCU codon. In some embodiments, at least 70%, at least 80%, at least 90%, at least 95%, at least 99%, or 100%, of the CTA codons in the nucleic acid coding sequence of a microbial Argonaute protein are replaced with a CAU codon. In some embodiments, at least 70%, at least 80%, at least 90%, at least 95%, at least 99%, or 100%, of the CTA codons in the nucleic acid coding sequence of a microbial Argonaute protein are replaced with a CGU codon. In some embodiments, at least 70%, at least 80%, at least 90%, at least 95%, at least 99%, or 100%, of the CTA codons in the nucleic acid coding sequence of a microbial Argonaute protein are replaced with a CUC codon. In some embodiments, at least 70%, at least 80%, at least 90%, at least 95%, at least 99%, or 100%, of the CTA codons in the nucleic acid coding sequence of a microbial Argonaute protein are replaced with a CCC codon. In some embodiments, at least 70%, at least 80%, at least 90%, at least 95%, at least 99%, or 100%, of the CTA codons in the nucleic acid coding sequence of a microbial Argonaute protein are replaced with a CAC codon. In some embodiments, at least 70%, at least 80%, at least 90%, at least 95%, at least 99%, or 100%, of the CTA codons in the nucleic acid coding sequence of a microbial Argonaute protein are replaced with a CGC codon. In some embodiments, at least 70%, at least 80%, at least 90%, at least 95%, at least 99%, or 100%, of the CTA codons in the nucleic acid coding sequence of a microbial Argonaute protein are replaced with a CUA codon. In some embodiments, at least 70%, at least 80%, at least 90%, at least 95%, at least 99%, or 100%, of the CTA codons in the nucleic acid coding sequence of a microbial Argonaute protein are replaced with a CCA codon. In some embodiments, at least 70%, at least 80%, at least 90%, at least 95%, at least 99%, or 100%, of the CTA codons in the nucleic acid coding sequence of a microbial Argonaute protein are replaced with a CAA codon. In some embodiments, at least 70%, at least 80%, at least 90%, at least 95%, at least 99%, or 100%, of the CTA codons in the nucleic acid coding sequence of a microbial Argonaute protein are replaced with a CGA codon. In some embodiments, at least 70%, at least 80%, at least 90%, at least 95%, at least 99%, or 100%, of the CTA codons in the nucleic acid coding sequence of a microbial Argonaute protein are replaced with a CUG codon. In some embodiments, at least 70%, at least 80%, at least 90%, at least 95%, at least 99%, or 100%, of the CTA codons in the nucleic acid coding sequence of a microbial Argonaute protein are replaced with a CCG codon. In some embodiments, at least 70%, at least 80%, at least 90%, at least 95%, at least 99%, or 100%, of the CTA codons in the nucleic acid coding sequence of a microbial Argonaute protein are replaced with a CAG codon. In some embodiments, at least 70%, at least 80%, at least 90%, at least 95%, at least 99%, or 100%, of the CTA codons in the nucleic acid coding sequence of a microbial Argonaute protein are replaced with a CGG codon. In some embodiments, at least 70%, at least 80%, at least 90%, at least 95%, at least 99%, or 100%, of the CTA codons in the nucleic acid coding sequence of a microbial Argonaute protein are replaced with an AUU codon. In some embodiments, at least 70%, at least 80%, at least 90%, at least 95%, at least 99%, or 100%, of the CTA codons in the nucleic acid coding sequence of a microbial Argonaute protein are replaced with an ACU codon. In some embodiments, at least 70%, at least 80%, at least 90%, at least 95%, at least 99%, or 100%, of the CTA codons in the nucleic acid coding sequence of a microbial Argonaute protein are replaced with an AAU codon. In some embodiments, at least 70%, at least 80%, at least 90%, at least 95%, at least 99%, or 100%, of the CTA codons in the nucleic acid coding sequence of a microbial Argonaute protein are replaced with an AGU codon. In some embodiments, at least 70%, at least 80%, at least 90%, at least 95%, at least 99%, or 100%, of the CTA codons in the nucleic acid coding sequence of a microbial Argonaute protein are replaced with an AUC codon. In some embodiments, at least 70%, at least 80%, at least 90%, at least 95%, at least 99%, or 100%, of the CTA codons in the nucleic acid coding sequence of a microbial Argonaute protein are replaced with an ACC codon. In some embodiments, at least 70%, at least 80%, at least 90%, at least 95%, at least 99%, or 100%, of the CTA codons in the nucleic acid coding sequence of a microbial Argonaute protein are replaced with an AAC codon. In some embodiments, at least 70%, at least 80%, at least 90%, at least 95%, at least 99%, or 100%, of the CTA codons in the nucleic acid coding sequence of a microbial Argonaute protein are replaced with an AGC codon. In some embodiments, at least 70%, at least 80%, at least 90%, at least 95%, at least 99%, or 100%, of the CTA codons in the nucleic acid coding sequence of a microbial Argonaute protein are replaced with an AUA codon. In some embodiments, at least 70%, at least 80%, at least 90%, at least 95%, at least 99%, or 100%, of the CTA codons in the nucleic acid coding sequence of a microbial Argonaute protein are replaced with an ACA codon. In some embodiments, at least 70%, at least 80%, at least 90%, at least 95%, at least 99%, or 100%, of the CTA codons in the nucleic acid coding sequence of a microbial Argonaute protein are replaced with an AAA codon. In some embodiments, at least 70%, at least 80%, at least 90%, at least 95%, at least 99%, or 100%, of the CTA codons in the nucleic acid coding sequence of a microbial Argonaute protein are replaced with an AGA codon. In some embodiments, at least 70%, at least 80%, at least 90%, at least 95%, at least 99%, or 100%, of the CTA codons in the nucleic acid coding sequence of a microbial Argonaute protein are replaced with an AUG codon. In some embodiments, at least 70%, at least 80%, at least 90%, at least 95%, at least 99%, or 100%, of the CTA codons in the nucleic acid coding sequence of a microbial Argonaute protein are replaced with an ACG codon. In some embodiments, at least 70%, at least 80%, at least 90%, at least 95%, at least 99%, or 100%, of the CTA codons in the nucleic acid coding sequence of a microbial Argonaute protein are replaced with an AAG codon. In some embodiments, at least 70%, at least 80%, at least 90%, at least 95%, at least 99%, or 100%, of the CTA codons in the nucleic acid coding sequence of a microbial Argonaute protein are replaced with an AGG codon. In some embodiments, at least 70%, at least 80%, at least 90%, at least 95%, at least 99%, or 100%, of the CTA codons in the nucleic acid coding sequence of a microbial Argonaute protein are replaced with a GUU codon. In some embodiments, at least 70%, at least 80%, at least 90%, at least 95%, at least 99%, or 100%, of the CTA codons in the nucleic acid coding sequence of a microbial Argonaute protein are replaced with a GCU codon. In some embodiments, at least 70%, at least 80%, at least 90%, at least 95%, at least 99%, or 100%, of the CTA codons in the nucleic acid coding sequence of a microbial Argonaute protein are replaced with a GAU codon. In some embodiments, at least 70%, at least 80%, at least 90%, at least 95%, at least 99%, or 100%, of the CTA codons in the nucleic acid coding sequence of a microbial Argonaute protein are replaced with a GGU codon. In some embodiments, at least 70%, at least 80%, at least 90%, at least 95%, at least 99%, or 100%, of the CTA codons in the nucleic acid coding sequence of a microbial Argonaute protein are replaced with a GUC codon. In some embodiments, at least 70%, at least 80%, at least 90%, at least 95%, at least 99%, or 100%, of the CTA codons in the nucleic acid coding sequence of a microbial Argonaute protein are replaced with a GCC codon. In some embodiments, at least 70%, at least 80%, at least 90%, at least 95%, at least 99%, or 100%, of the CTA codons in the nucleic acid coding sequence of a microbial Argonaute protein are replaced with a GAC codon. In some embodiments, at least 70%, at least 80%, at least 90%, at least 95%, at least 99%, or 100%, of the CTA codons in the nucleic acid coding sequence of a microbial Argonaute protein are replaced with a GGC codon. In some embodiments, at least 70%, at least 80%, at least 90%, at least 95%, at least 99%, or 100%, of the CTA codons in the nucleic acid coding sequence of a microbial Argonaute protein are replaced with a GUA codon. In some embodiments, at least 70%, at least 80%, at least 90%, at least 95%, at least 99%, or 100%, of the CTA codons in the nucleic acid coding sequence of a microbial Argonaute protein are replaced with a GCA codon. In some embodiments, at least 70%, at least 80%, at least 90%, at least 95%, at least 99%, or 100%, of the CTA codons in the nucleic acid coding sequence of a microbial Argonaute protein are replaced with a GAA codon. In some embodiments, at least 70%, at least 80%, at least 90%, at least 95%, at least 99%, or 100%, of the CTA codons in the nucleic acid coding sequence of a microbial Argonaute protein are replaced with a GGA codon. In some embodiments, at least 70%, at least 80%, at least 90%, at least 95%, at least 99%, or 100%, of the CTA codons in the nucleic acid coding sequence of a microbial Argonaute protein are replaced with a GUG codon. In some embodiments, at least 70%, at least 80%, at least 90%, at least 95%, at least 99%, or 100%, of the CTA codons in the nucleic acid coding sequence of a microbial Argonaute protein are replaced with a GCG codon. In some embodiments, at least 70%, at least 80%, at least 90%, at least 95%, at least 99%, or 100%, of the CTA codons in the nucleic acid coding sequence of a microbial Argonaute protein are replaced with a GAG codon. In some embodiments, at least 70%, at least 80%, at least 90%, at least 95%, at least 99%, or 100%, of the CTA codons in the nucleic acid coding sequence of a microbial Argonaute protein are replaced with a GGG codon.

In some embodiments, at least 70%, at least 80%, at least 90%, at least 95%, at least 99%, or 100%, of the TTA codons in the nucleic acid coding sequence of a microbial Argonaute protein are replaced with a human preferred codon. In some embodiments, at least 70%, at least 80%, at least 90%, at least 95%, at least 99%, or 100%, of the TTA codons in the nucleic acid coding sequence of a microbial Argonaute protein are replaced with a UUU codon. In some embodiments, at least 70%, at least 80%, at least 90%, at least 95%, at least 99%, or 100%, of the TTA codons in the nucleic acid coding sequence of a microbial Argonaute protein are replaced with a UCU codon. In some embodiments, at least 70%, at least 80%, at least 90%, at least 95%, at least 99%, or 100%, of the TTA codons in the nucleic acid coding sequence of a microbial Argonaute protein are replaced with a UAU codon. In some embodiments, at least 70%, at least 80%, at least 90%, at least 95%, at least 99%, or 100%, of the TTA codons in the nucleic acid coding sequence of a microbial Argonaute protein are replaced with a UGU codon. In some embodiments, at least 70%, at least 80%, at least 90%, at least 95%, at least 99%, or 100%, of the TTA codons in the nucleic acid coding sequence of a microbial Argonaute protein are replaced with a UUC codon. In some embodiments, at least 70%, at least 80%, at least 90%, at least 95%, at least 99%, or 100%, of the TTA codons in the nucleic acid coding sequence of a microbial Argonaute protein are replaced with a UCC codon. In some embodiments, at least 70%, at least 80%, at least 90%, at least 95%, at least 99%, or 100%, of the TTA codons in the nucleic acid coding sequence of a microbial Argonaute protein are replaced with a UAC codon. In some embodiments, at least 70%, at least 80%, at least 90%, at least 95%, at least 99%, or 100%, of the TTA codons in the nucleic acid coding sequence of a microbial Argonaute protein are replaced with a UGC codon. In some embodiments, at least 70%, at least 80%, at least 90%, at least 95%, at least 99%, or 100%, of the TTA codons in the nucleic acid coding sequence of a microbial Argonaute protein are replaced with a UUA codon. In some embodiments, at least 70%, at least 80%, at least 90%, at least 95%, at least 99%, or 100%, of the TTA codons in the nucleic acid coding sequence of a microbial Argonaute protein are replaced with a UCA codon. In some embodiments, at least 70%, at least 80%, at least 90%, at least 95%, at least 99%, or 100%, of the TTA codons in the nucleic acid coding sequence of a microbial Argonaute protein are replaced with a UUG codon. In some embodiments, at least 70%, at least 80%, at least 90%, at least 95%, at least 99%, or 100%, of the TTA codons in the nucleic acid coding sequence of a microbial Argonaute protein are replaced with a UCG codon. In some embodiments, at least 70%, at least 80%, at least 90%, at least 95%, at least 99%, or 100%, of the TTA codons in the nucleic acid coding sequence of a microbial Argonaute protein are replaced with a UGG codon. In some embodiments, at least 70%, at least 80%, at least 90%, at least 95%, at least 99%, or 100%, of the TTA codons in the nucleic acid coding sequence of a microbial Argonaute protein are replaced with a CUU codon. In some embodiments, at least 70%, at least 80%, at least 90%, at least 95%, at least 99%, or 100%, of the TTA codons in the nucleic acid coding sequence of a microbial Argonaute protein are replaced with a CCU codon. In some embodiments, at least 70%, at least 80%, at least 90%, at least 95%, at least 99%, or 100%, of the TTA codons in the nucleic acid coding sequence of a microbial Argonaute protein are replaced with a CAU codon. In some embodiments, at least 70%, at least 80%, at least 90%, at least 95%, at least 99%, or 100%, of the TTA codons in the nucleic acid coding sequence of a microbial Argonaute protein are replaced with a CGU codon. In some embodiments, at least 70%, at least 80%, at least 90%, at least 95%, at least 99%, or 100%, of the TTA codons in the nucleic acid coding sequence of a microbial Argonaute protein are replaced with a CUC codon. In some embodiments, at least 70%, at least 80%, at least 90%, at least 95%, at least 99%, or 100%, of the TTA codons in the nucleic acid coding sequence of a microbial Argonaute protein are replaced with a CCC codon. In some embodiments, at least 70%, at least 80%, at least 90%, at least 95%, at least 99%, or 100%, of the TTA codons in the nucleic acid coding sequence of a microbial Argonaute protein are replaced with a CAC codon. In some embodiments, at least 70%, at least 80%, at least 90%, at least 95%, at least 99%, or 100%, of the TTA codons in the nucleic acid coding sequence of a microbial Argonaute protein are replaced with a CGC codon. In some embodiments, at least 70%, at least 80%, at least 90%, at least 95%, at least 99%, or 100%, of the TTA codons in the nucleic acid coding sequence of a microbial Argonaute protein are replaced with a CUA codon. In some embodiments, at least 70%, at least 80%, at least 90%, at least 95%, at least 99%, or 100%, of the TTA codons in the nucleic acid coding sequence of a microbial Argonaute protein are replaced with a CCA codon. In some embodiments, at least 70%, at least 80%, at least 90%, at least 95%, at least 99%, or 100%, of the TTA codons in the nucleic acid coding sequence of a microbial Argonaute protein are replaced with a CAA codon. In some embodiments, at least 70%, at least 80%, at least 90%, at least 95%, at least 99%, or 100%, of the TTA codons in the nucleic acid coding sequence of a microbial Argonaute protein are replaced with a CGA codon. In some embodiments, at least 70%, at least 80%, at least 90%, at least 95%, at least 99%, or 100%, of the TTA codons in the nucleic acid coding sequence of a microbial Argonaute protein are replaced with a CUG codon. In some embodiments, at least 70%, at least 80%, at least 90%, at least 95%, at least 99%, or 100%, of the TTA codons in the nucleic acid coding sequence of a microbial Argonaute protein are replaced with a CCG codon. In some embodiments, at least 70%, at least 80%, at least 90%, at least 95%, at least 99%, or 100%, of the TTA codons in the nucleic acid coding sequence of a microbial Argonaute protein are replaced with a CAG codon. In some embodiments, at least 70%, at least 80%, at least 90%, at least 95%, at least 99%, or 100%, of the TTA codons in the nucleic acid coding sequence of a microbial Argonaute protein are replaced with a CGG codon. In some embodiments, at least 70%, at least 80%, at least 90%, at least 95%, at least 99%, or 100%, of the TTA codons in the nucleic acid coding sequence of a microbial Argonaute protein are replaced with an AUU codon. In some embodiments, at least 70%, at least 80%, at least 90%, at least 95%, at least 99%, or 100%, of the TTA codons in the nucleic acid coding sequence of a microbial Argonaute protein are replaced with an ACU codon. In some embodiments, at least 70%, at least 80%, at least 90%, at least 95%, at least 99%, or 100%, of the TTA codons in the nucleic acid coding sequence of a microbial Argonaute protein are replaced with an AAU codon. In some embodiments, at least 70%, at least 80%, at least 90%, at least 95%, at least 99%, or 100%, of the TTA codons in the nucleic acid coding sequence of a microbial Argonaute protein are replaced with an AGU codon. In some embodiments, at least 70%, at least 80%, at least 90%, at least 95%, at least 99%, or 100%, of the TTA codons in the nucleic acid coding sequence of a microbial Argonaute protein are replaced with an AUC codon. In some embodiments, at least 70%, at least 80%, at least 90%, at least 95%, at least 99%, or 100%, of the TTA codons in the nucleic acid coding sequence of a microbial Argonaute protein are replaced with an ACC codon. In some embodiments, at least 70%, at least 80%, at least 90%, at least 95%, at least 99%, or 100%, of the TTA codons in the nucleic acid coding sequence of a microbial Argonaute protein are replaced with an AAC codon. In some embodiments, at least 70%, at least 80%, at least 90%, at least 95%, at least 99%, or 100%, of the TTA codons in the nucleic acid coding sequence of a microbial Argonaute protein are replaced with an AGC codon. In some embodiments, at least 70%, at least 80%, at least 90%, at least 95%, at least 99%, or 100%, of the TTA codons in the nucleic acid coding sequence of a microbial Argonaute protein are replaced with an AUA codon. In some embodiments, at least 70%, at least 80%, at least 90%, at least 95%, at least 99%, or 100%, of the TTA codons in the nucleic acid coding sequence of a microbial Argonaute protein are replaced with an ACA codon. In some embodiments, at least 70%, at least 80%, at least 90%, at least 95%, at least 99%, or 100%, of the TTA codons in the nucleic acid coding sequence of a microbial Argonaute protein are replaced with a AAA codon. In some embodiments, at least 70%, at least 80%, at least 90%, at least 95%, at least 99%, or 100%, of the TTA codons in the nucleic acid coding sequence of a microbial Argonaute protein are replaced with an AGA codon. In some embodiments, at least 70%, at least 80%, at least 90%, at least 95%, at least 99%, or 100%, of the TTA codons in the nucleic acid coding sequence of a microbial Argonaute protein are replaced with an AUG codon. In some embodiments, at least 70%, at least 80%, at least 90%, at least 95%, at least 99%, or 100%, of the TTA codons in the nucleic acid coding sequence of a microbial Argonaute protein are replaced with an ACG codon. In some embodiments, at least 70%, at least 80%, at least 90%, at least 95%, at least 99%, or 100%, of the TTA codons in the nucleic acid coding sequence of a microbial Argonaute protein are replaced with an AAG codon. In some embodiments, at least 70%, at least 80%, at least 90%, at least 95%, at least 99%, or 100%, of the TTA codons in the nucleic acid coding sequence of a microbial Argonaute protein are replaced with an AGG codon. In some embodiments, at least 70%, at least 80%, at least 90%, at least 95%, at least 99%, or 100%, of the TTA codons in the nucleic acid coding sequence of a microbial Argonaute protein are replaced with a GUU codon. In some embodiments, at least 70%, at least 80%, at least 90%, at least 95%, at least 99%, or 100%, of the TTA codons in the nucleic acid coding sequence of a microbial Argonaute protein are replaced with a GCU codon. In some embodiments, at least 70%, at least 80%, at least 90%, at least 95%, at least 99%, or 100%, of the TTA codons in the nucleic acid coding sequence of a microbial Argonaute protein are replaced with a GAU codon. In some embodiments, at least 70%, at least 80%, at least 90%, at least 95%, at least 99%, or 100%, of the TTA codons in the nucleic acid coding sequence of a microbial Argonaute protein are replaced with a GGU codon. In some embodiments, at least 70%, at least 80%, at least 90%, at least 95%, at least 99%, or 100%, of the TTA codons in the nucleic acid coding sequence of a microbial Argonaute protein are replaced with a GUC codon. In some embodiments, at least 70%, at least 80%, at least 90%, at least 95%, at least 99%, or 100%, of the TTA codons in the nucleic acid coding sequence of a microbial Argonaute protein are replaced with a GCC codon. In some embodiments, at least 70%, at least 80%, at least 90%, at least 95%, at least 99%, or 100%, of the TTA codons in the nucleic acid coding sequence of a microbial Argonaute protein are replaced with a GAC codon. In some embodiments, at least 70%, at least 80%, at least 90%, at least 95%, at least 99%, or 100%, of the TTA codons in the nucleic acid coding sequence of a microbial Argonaute protein are replaced with a GGC codon. In some embodiments, at least 70%, at least 80%, at least 90%, at least 95%, at least 99%, or 100%, of the TTA codons in the nucleic acid coding sequence of a microbial Argonaute protein are replaced with a GUA codon. In some embodiments, at least 70%, at least 80%, at least 90%, at least 95%, at least 99%, or 100%, of the TTA codons in the nucleic acid coding sequence of a microbial Argonaute protein are replaced with a GCA codon. In some embodiments, at least 70%, at least 80%, at least 90%, at least 95%, at least 99%, or 100%, of the TTA codons in the nucleic acid coding sequence of a microbial Argonaute protein are replaced with a GAA codon. In some embodiments, at least 70%, at least 80%, at least 90%, at least 95%, at least 99%, or 100%, of the TTA codons in the nucleic acid coding sequence of a microbial Argonaute protein are replaced with a GGA codon. In some embodiments, at least 70%, at least 80%, at least 90%, at least 95%, at least 99%, or 100%, of the TTA codons in the nucleic acid coding sequence of a microbial Argonaute protein are replaced with a GUG codon. In some embodiments, at least 70%, at least 80%, at least 90%, at least 95%, at least 99%, or 100%, of the TTA codons in the nucleic acid coding sequence of a microbial Argonaute protein are replaced with a GCG codon. In some embodiments, at least 70%, at least 80%, at least 90%, at least 95%, at least 99%, or 100%, of the TTA codons in the nucleic acid coding sequence of a microbial Argonaute protein are replaced with a GAG codon. In some embodiments, at least 70%, at least 80%, at least 90%, at least 95%, at least 99%, or 100%, of the TTA codons in the nucleic acid coding sequence of a microbial Argonaute protein are replaced with a GGG codon.

In some embodiments, at least 70%, at least 80%, at least 90%, at least 95%, at least 99%, or 100%, of the GCG codons in the nucleic acid coding sequence of a microbial Argonaute protein are replaced with a human preferred codon. In some embodiments, at least 70%, at least 80%, at least 90%, at least 95%, at least 99%, or 100%, of the GCG codons in the nucleic acid coding sequence of a microbial Argonaute protein are replaced with a UUU codon. In some embodiments, at least 70%, at least 80%, at least 90%, at least 95%, at least 99%, or 100%, of the GCG codons in the nucleic acid coding sequence of a microbial Argonaute protein are replaced with a UCU codon. In some embodiments, at least 70%, at least 80%, at least 90%, at least 95%, at least 99%, or 100%, of the GCG codons in the nucleic acid coding sequence of a microbial Argonaute protein are replaced with a UAU codon. In some embodiments, at least 70%, at least 80%, at least 90%, at least 95%, at least 99%, or 100%, of the GCG codons in the nucleic acid coding sequence of a microbial Argonaute protein are replaced with a UGU codon. In some embodiments, at least 70%, at least 80%, at least 90%, at least 95%, at least 99%, or 100%, of the GCG codons in the nucleic acid coding sequence of a microbial Argonaute protein are replaced with a UUC codon. In some embodiments, at least 70%, at least 80%, at least 90%, at least 95%, at least 99%, or 100%, of the GCG codons in the nucleic acid coding sequence of a microbial Argonaute protein are replaced with a UCC codon. In some embodiments, at least 70%, at least 80%, at least 90%, at least 95%, at least 99%, or 100%, of the GCG codons in the nucleic acid coding sequence of a microbial Argonaute protein are replaced with a UAC codon. In some embodiments, at least 70%, at least 80%, at least 90%, at least 95%, at least 99%, or 100%, of the GCG codons in the nucleic acid coding sequence of a microbial Argonaute protein are replaced with a UGC codon. In some embodiments, at least 70%, at least 80%, at least 90%, at least 95%, at least 99%, or 100%, of the GCG codons in the nucleic acid coding sequence of a microbial Argonaute protein are replaced with a UUA codon. In some embodiments, at least 70%, at least 80%, at least 90%, at least 95%, at least 99%, or 100%, of the GCG codons in the nucleic acid coding sequence of a microbial Argonaute protein are replaced with a UCA codon. In some embodiments, at least 70%, at least 80%, at least 90%, at least 95%, at least 99%, or 100%, of the GCG codons in the nucleic acid coding sequence of a microbial Argonaute protein are replaced with a UUG codon. In some embodiments, at least 70%, at least 80%, at least 90%, at least 95%, at least 99%, or 100%, of the GCG codons in the nucleic acid coding sequence of a microbial Argonaute protein are replaced with a UCG codon. In some embodiments, at least 70%, at least 80%, at least 90%, at least 95%, at least 99%, or 100%, of the GCG codons in the nucleic acid coding sequence of a microbial Argonaute protein are replaced with a UGG codon. In some embodiments, at least 70%, at least 80%, at least 90%, at least 95%, at least 99%, or 100%, of the GCG codons in the nucleic acid coding sequence of a microbial Argonaute protein are replaced with a CUU codon. In some embodiments, at least 70%, at least 80%, at least 90%, at least 95%, at least 99%, or 100%, of the GCG codons in the nucleic acid coding sequence of a microbial Argonaute protein are replaced with a CCU codon. In some embodiments, at least 70%, at least 80%, at least 90%, at least 95%, at least 99%, or 100%, of the GCG codons in the nucleic acid coding sequence of a microbial Argonaute protein are replaced with a CAU codon. In some embodiments, at least 70%, at least 80%, at least 90%, at least 95%, at least 99%, or 100%, of the GCG codons in the nucleic acid coding sequence of a microbial Argonaute protein are replaced with a CGU codon. In some embodiments, at least 70%, at least 80%, at least 90%, at least 95%, at least 99%, or 100%, of the GCG codons in the nucleic acid coding sequence of a microbial Argonaute protein are replaced with a CUC codon. In some embodiments, at least 70%, at least 80%, at least 90%, at least 95%, at least 99%, or 100%, of the GCG codons in the nucleic acid coding sequence of a microbial Argonaute protein are replaced with a CCC codon. In some embodiments, at least 70%, at least 80%, at least 90%, at least 95%, at least 99%, or 100%, of the GCG codons in the nucleic acid coding sequence of a microbial Argonaute protein are replaced with a CAC codon. In some embodiments, at least 70%, at least 80%, at least 90%, at least 95%, at least 99%, or 100%, of the GCG codons in the nucleic acid coding sequence of a microbial Argonaute protein are replaced with a CGC codon. In some embodiments, at least 70%, at least 80%, at least 90%, at least 95%, at least 99%, or 100%, of the GCG codons in the nucleic acid coding sequence of a microbial Argonaute protein are replaced with a CUA codon. In some embodiments, at least 70%, at least 80%, at least 90%, at least 95%, at least 99%, or 100%, of the GCG codons in the nucleic acid coding sequence of a microbial Argonaute protein are replaced with a CCA codon. In some embodiments, at least 70%, at least 80%, at least 90%, at least 95%, at least 99%, or 100%, of the GCG codons in the nucleic acid coding sequence of a microbial Argonaute protein are replaced with a CAA codon. In some embodiments, at least 70%, at least 80%, at least 90%, at least 95%, at least 99%, or 100%, of the GCG codons in the nucleic acid coding sequence of a microbial Argonaute protein are replaced with a CGA codon. In some embodiments, at least 70%, at least 80%, at least 90%, at least 95%, at least 99%, or 100%, of the GCG codons in the nucleic acid coding sequence of a microbial Argonaute protein are replaced with a CUG codon. In some embodiments, at least 70%, at least 80%, at least 90%, at least 95%, at least 99%, or 100%, of the GCG codons in the nucleic acid coding sequence of a microbial Argonaute protein are replaced with a CCG codon. In some embodiments, at least 70%, at least 80%, at least 90%, at least 95%, at least 99%, or 100%, of the GCG codons in the nucleic acid coding sequence of a microbial Argonaute protein are replaced with a CAG codon. In some embodiments, at least 70%, at least 80%, at least 90%, at least 95%, at least 99%, or 100%, of the GCG codons in the nucleic acid coding sequence of a microbial Argonaute protein are replaced with a CGG codon. In some embodiments, at least 70%, at least 80%, at least 90%, at least 95%, at least 99%, or 100%, of the GCG codons in the nucleic acid coding sequence of a microbial Argonaute protein are replaced with an AUU codon. In some embodiments, at least 70%, at least 80%, at least 90%, at least 95%, at least 99%, or 100%, of the GCG codons in the nucleic acid coding sequence of a microbial Argonaute protein are replaced with an ACU codon. In some embodiments, at least 70%, at least 80%, at least 90%, at least 95%, at least 99%, or 100%, of the GCG codons in the nucleic acid coding sequence of a microbial Argonaute protein are replaced with an AAU codon. In some embodiments, at least 70%, at least 80%, at least 90%, at least 95%, at least 99%, or 100%, of the GCG codons in the nucleic acid coding sequence of a microbial Argonaute protein are replaced with an AGU codon. In some embodiments, at least 70%, at least 80%, at least 90%, at least 95%, at least 99%, or 100%, of the GCG codons in the nucleic acid coding sequence of a microbial Argonaute protein are replaced with an AUC codon. In some embodiments, at least 70%, at least 80%, at least 90%, at least 95%, at least 99%, or 100%, of the GCG codons in the nucleic acid coding sequence of a microbial Argonaute protein are replaced with an ACC codon. In some embodiments, at least 70%, at least 80%, at least 90%, at least 95%, at least 99%, or 100%, of the GCG codons in the nucleic acid coding sequence of a microbial Argonaute protein are replaced with an AAC codon. In some embodiments, at least 70%, at least 80%, at least 90%, at least 95%, at least 99%, or 100%, of the GCG codons in the nucleic acid coding sequence of a microbial Argonaute protein are replaced with an AGC codon. In some embodiments, at least 70%, at least 80%, at least 90%, at least 95%, at least 99%, or 100%, of the GCG codons in the nucleic acid coding sequence of a microbial Argonaute protein are replaced with an AUA codon. In some embodiments, at least 70%, at least 80%, at least 90%, at least 95%, at least 99%, or 100%, of the GCG codons in the nucleic acid coding sequence of a microbial Argonaute protein are replaced with an ACA codon. In some embodiments, at least 70%, at least 80%, at least 90%, at least 95%, at least 99%, or 100%, of the GCG codons in the nucleic acid coding sequence of a microbial Argonaute protein are replaced with a AAA codon. In some embodiments, at least 70%, at least 80%, at least 90%, at least 95%, at least 99%, or 100%, of the GCG codons in the nucleic acid coding sequence of a microbial Argonaute protein are replaced with an AGA codon. In some embodiments, at least 70%, at least 80%, at least 90%, at least 95%, at least 99%, or 100%, of the GCG codons in the nucleic acid coding sequence of a microbial Argonaute protein are replaced with an AUG codon. In some embodiments, at least 70%, at least 80%, at least 90%, at least 95%, at least 99%, or 100%, of the GCG codons in the nucleic acid coding sequence of a microbial Argonaute protein are replaced with an ACG codon. In some embodiments, at least 70%, at least 80%, at least 90%, at least 95%, at least 99%, or 100%, of the GCG codons in the nucleic acid coding sequence of a microbial Argonaute protein are replaced with an AAG codon. In some embodiments, at least 70%, at least 80%, at least 90%, at least 95%, at least 99%, or 100%, of the GCG codons in the nucleic acid coding sequence of a microbial Argonaute protein are replaced with an AGG codon. In some embodiments, at least 70%, at least 80%, at least 90%, at least 95%, at least 99%, or 100%, of the GCG codons in the nucleic acid coding sequence of a microbial Argonaute protein are replaced with a GUU codon. In some embodiments, at least 70%, at least 80%, at least 90%, at least 95%, at least 99%, or 100%, of the GCG codons in the nucleic acid coding sequence of a microbial Argonaute protein are replaced with a GCU codon. In some embodiments, at least 70%, at least 80%, at least 90%, at least 95%, at least 99%, or 100%, of the GCG codons in the nucleic acid coding sequence of a microbial Argonaute protein are replaced with a GAU codon. In some embodiments, at least 70%, at least 80%, at least 90%, at least 95%, at least 99%, or 100%, of the GCG codons in the nucleic acid coding sequence of a microbial Argonaute protein are replaced with a GGU codon. In some embodiments, at least 70%, at least 80%, at least 90%, at least 95%, at least 99%, or 100%, of the GCG codons in the nucleic acid coding sequence of a microbial Argonaute protein are replaced with a GUC codon. In some embodiments, at least 70%, at least 80%, at least 90%, at least 95%, at least 99%, or 100%, of the GCG codons in the nucleic acid coding sequence of a microbial Argonaute protein are replaced with a GCC codon. In some embodiments, at least 70%, at least 80%, at least 90%, at least 95%, at least 99%, or 100%, of the GCG codons in the nucleic acid coding sequence of a microbial Argonaute protein are replaced with a GAC codon. In some embodiments, at least 70%, at least 80%, at least 90%, at least 95%, at least 99%, or 100%, of the GCG codons in the nucleic acid coding sequence of a microbial Argonaute protein are replaced with a GGC codon. In some embodiments, at least 70%, at least 80%, at least 90%, at least 95%, at least 99%, or 100%, of the GCG codons in the nucleic acid coding sequence of a microbial Argonaute protein are replaced with a GUA codon. In some embodiments, at least 70%, at least 80%, at least 90%, at least 95%, at least 99%, or 100%, of the GCG codons in the nucleic acid coding sequence of a microbial Argonaute protein are replaced with a GCA codon. In some embodiments, at least 70%, at least 80%, at least 90%, at least 95%, at least 99%, or 100%, of the GCG codons in the nucleic acid coding sequence of a microbial Argonaute protein are replaced with a GAA codon. In some embodiments, at least 70%, at least 80%, at least 90%, at least 95%, at least 99%, or 100%, of the GCG codons in the nucleic acid coding sequence of a microbial Argonaute protein are replaced with a GGA codon. In some embodiments, at least 70%, at least 80%, at least 90%, at least 95%, at least 99%, or 100%, of the GCG codons in the nucleic acid coding sequence of a microbial Argonaute protein are replaced with a GUG codon. In some embodiments, at least 70%, at least 80%, at least 90%, at least 95%, at least 99%, or 100%, of the GCG codons in the nucleic acid coding sequence of a microbial Argonaute protein are replaced with a GCG codon. In some embodiments, at least 70%, at least 80%, at least 90%, at least 95%, at least 99%, or 100%, of the GCG codons in the nucleic acid coding sequence of a microbial Argonaute protein are replaced with a GAG codon. In some embodiments, at least 70%, at least 80%, at least 90%, at least 95%, at least 99%, or 100%, of the GCG codons in the nucleic acid coding sequence of a microbial Argonaute protein are replaced with a GGG codon.

In certain embodiments, a functional fragment of a microbial Argonaute polypeptide comprises a polypeptide sequence comprising at least 30, at least 50, at least 100, at least 200, at least 300, at least 400, at least 500, at least 600, at least 700 or at least 800 amino acids having at least 75%, at least 80%, at least 85%, at least 90%, at least 95%, at least 98%, at least 99%, or 100%, 70% to 100% identity, 80% to 100% identity, 90% to 100% identity or 95% to 100% identity to the sequence of the prokaryotic Argonaute polypeptide.

In certain embodiments, an ANAGO comprises a polypeptide encoded by portion of a nucleotide sequence of SEQ ID NO:1, SEQ ID NO:2, SEQ ID NO:3, or SEQ ID NO:4, wherein the ANAGO, when expressed in a eukaryotic cell, comprises an ability to edit a target nucleic acid sequence within the eukaryotic cell. In certain embodiments, an ANAGO comprises a polypeptide encoded by portion of the nucleotide sequence of SEQ ID NO:2, SEQ ID NO:3 or SEQ ID NO:4, wherein the ANAGO, when expressed in a eukaryotic cell, comprises an ability to edit a target nucleic acid sequence (target site) within the eukaryotic cell.

In some embodiments, a nucleic acid described herein encodes all or a portion of an ANAGO, non-limiting examples of which include a portion that is 1 to 903 amino acids in length, or at least 50, at least 100, at least 200, at least 300, or at least 500 amino acids in length. In some embodiments, an ANAGO derived and reengineered from the DNA coding sequence of all or a portion of an Argonaute protein/polypeptide such as NgAgo, PfAgo, TtAgo, or MjAgo having nuclease activity, conserves or retains the nuclease activity and/or comprises the ability to insert a heterologous nucleic acid sequence into the genome of a living mammalian cell at a specific targeted locus. In some embodiments, a nucleic acid that encodes an ANAGO, is 70-100%, 70-80%, 80-90%, 90-100%, 80-85%, 85-95%, 90-95%, 95-100%, 80% to 100%, at least 70%, at least 80%, at least 81%, at least 82%, at least 85%, at least 90% or at least 95% identical to the nucleic acid having sequence of SEQ ID NO:1, SEQ ID NO:2, SEQ ID NO:3 or SEQ ID NO:4.

In some embodiments, a nucleic acid that encodes all or a portion of an ANAGO has a nucleic acid sequence having 70-100% or 80% to 100% identity to the nucleic acid sequence of SEQ ID NO:1, SEQ ID NO:2, SEQ ID NO:3, or SEQ ID NO:4. In certain embodiments, a nucleic acid is at least 80%, at least 81%, at least 82%, at least 85%, at least 90% or at least 95% identical to the nucleic acid sequence of SEQ ID NO:1, SEQ ID NO:2, SEQ ID NO:3, or SEQ ID NO:4. A nucleic acid described herein is often not a naturally occurring nucleic acid and is often not found in nature. In certain embodiments, a nucleic acid described herein is a synthetic nucleic acid. A synthetic nucleic acid refers to a nucleic acid sequence that is designed by the hand of man and is not found in nature.

In some embodiments, a nucleic acid that encodes all or a portion of an ANAGO is a nucleic acid that comprises or consists of 50 to 2666 contiguous nucleotides (nt) of the nucleic acid sequence of SEQ ID NO:1, SEQ ID NO:2, SEQ ID NO:3, or SEQ ID NO:4. In certain embodiments, a nucleic acid comprises or consists of at least 50, at least 100, at least 500, at least 750, at least 1000, at least 1500, at least 1750 or at least 2000 contiguous nucleotides of the nucleic acid sequence of SEQ ID NO:1, SEQ ID NO:2, SEQ ID NO:3, or SEQ ID NO:4. In some embodiments, a nucleic acid or synthetic nucleic acid described herein consists of, or comprises, the nucleic acid sequence of SEQ ID NO:1, SEQ ID NO:2, SEQ ID NO:3, or SEQ ID NO:4.

In certain embodiments, a nucleic acid or synthetic nucleic acid described herein comprises a nucleic acid sequence that is 100 to 3000 nucleotides in length having 70-100%, or 80% to 100% identity to the nucleic acid sequence of SEQ ID NO:1, SEQ ID NO:2, SEQ ID NO:3, or SEQ ID NO:4. In certain embodiments, a nucleic acid or synthetic nucleic acid described herein comprises a first nucleic acid sequence that is at least 100, at least 500, at least 750, at least 1000, at least 1500, at least 1750 or at least 2000 nucleotides in length, where the first nucleic acid has at least 70%, at least 80%, at least 81%, at least 82%, at least 85%, at least 90% or at least 95% identity to the nucleic acid sequence of SEQ ID NO:1, SEQ ID NO:2, SEQ ID NO:3, or SEQ ID NO:4.

In certain embodiments, a nucleic acid is configured to express a polypeptide in a mammalian cell. A nucleic acid that is configured to express a polypeptide (e.g., an ANAGO) comprises one or more nucleic acid regulatory sequences that direct the expression of a polypeptide in a cell. Accordingly, a nucleic acid that is configured to express a desired polypeptide, such as ANAGO, may include one or more of a coding region that encodes the desired protein, such as ANAGO, one or more suitable promoters operably linked to the coding region, a translation initiation sequence, a start codon, a stop codon, a polyA signal sequence, a leader sequence, a nuclear localization sequence, and the like. In certain embodiments, a nucleic acid comprises a sequence that encodes a nuclear localization signal (NLS) sequence. Any suitable NLS sequence can be used. One non-limiting example of an NLS sequence is SV40 nuclear localization signal (NLS) sequence. In certain embodiments, a nucleic acid is configured to express an ANAGO, or functional fragment thereof.

A target sequence refers to a specific location (a specific nucleic acid sequence) within the genome of an organism or a cell that one intends to modify using a composition or method described herein. In some embodiments, a target sequence is a nucleic acid located within a genome of a cell or an organism. In some embodiments, a target sequence comprises RNA. In certain embodiments, a target sequence comprises DNA. In some embodiments, a target sequence contains 1 or more nucleotides in length. In some embodiments, a target sequence can be as long as a few thousands or even millions base pairs in length, if it is in a linear contiguous DNA sequence in a chromosome. In certain embodiments, a target sequence is 1000 to 10,000, 5000-10,000, 1000 to 5000, 500 to 1000, 700-1000, 500-700, 100 to 900, 100 to 500, 100 to 300, 100 to 200, 50 to 100, 10 to 100, 200-300, 300-400, 400-500, 500-600, 600-700, 700-800, 800-900, 900-1000, 1000-2000, 2000-3000, 3000-4000, 4000-5000, 5000-6000, 6000-7000, 7000-8000, 8000-9000, 9000-10,000, 10-50, 16 to 50, 16 to 30, 16 to 20, 18 to 50, 18 to 30, 18 to 28, 18 to 25, 18 to 26, 18-20, 19-50, 19-30, 19 to 26, 19 to 25, 19-20, 20 to 30, 20 to 25, 20 to 24, 21 to 24, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any number in a range bounded by any of the above values of nucleotides (nt) or base pairs (bp) in length and may be located within a gene, exon, intron or any suitable portion of a genome. Any nucleotide within a target sequence or any portion of a target sequence can be modified by a method described herein. Any number of nucleotides within a target sequence may be deleted, mutated or replaced, for example by a desired sequence (e.g., an insert sequence of a donor sequence). In some embodiments, one or more nucleotides or a desired sequence are inserted into a target sequence by a method described herein. In certain embodiments, a target sequence provides a nucleic acid sequence that is complementary or identical to a guide oligonucleotide, or portion thereof, for example when the guide oligonucleotide is used in an experiment for comparison purpose as described below. In certain embodiments a target sequence provides a nucleic acid sequence that is complementary or identical to a 5′ and/or 3′ flanking regions of a donor sequence.

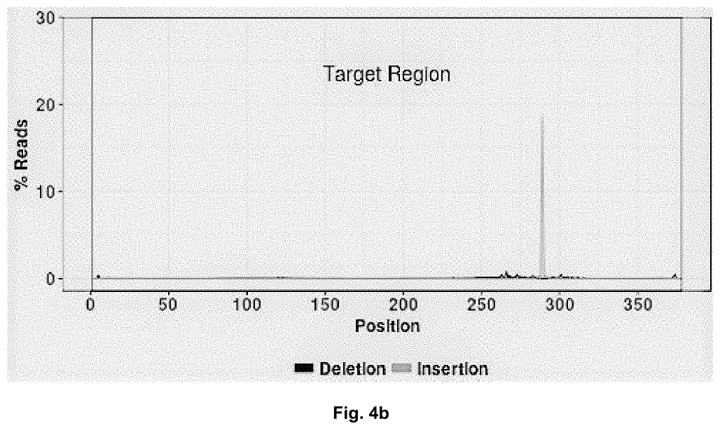

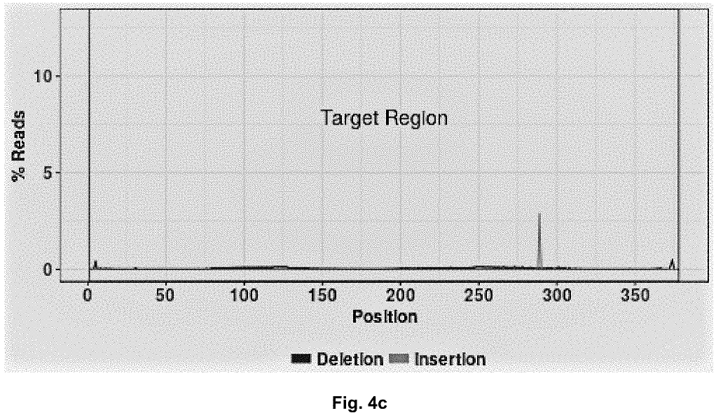

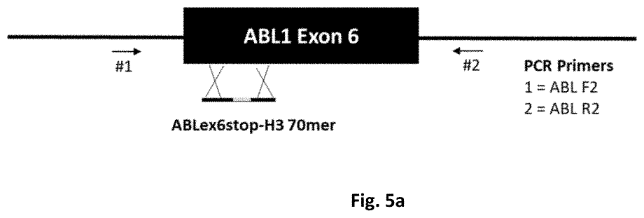

Although a guide oligonucleotide is not needed in the ANAGO induced gene editing technology, a guide molecule was used in some of the experiments described in FIG. 2b and FIG. 4b, solely for comparing the results in the experiment where a guide molecule was not used. A guide oligonucleotide often comprises a nucleic acid sequence that is 80% to 100% identical to the target site. A guide oligonucleotide is sometimes a nucleic acid that is 18 to 30 bases in length. Without being limited to theory, an ANAGO described herein can utilize a guide oligonucleotide to cut the genomic DNA of an organism or cell at a specific target sequence that is defined by the sequence of a guide oligonucleotide. In certain embodiments, an ANAGO cleaves a target nucleic acid sequence anywhere within a sequence defined by a guide oligonucleotide. In some embodiments, an ANAGO cleaves a target site at a location defined by any one of the first 10 nucleotides (5′-nucleotides) of a guide oligonucleotide. When both a guide oligonucleotide and a donor nucleic acid are present, an ANAGO will proceed to replace the targeted sequence with a donor nucleic acid into the genome of a cell at a target site defined by the guide oligonucleotide. If a donor sequence is not present, an ANAGO loaded with a guide oligonucleotide, will often cleave a target site defined by the guide oligonucleotide sequence. This process often results in the introduction of one or more single nucleotide mutations introduced at the target site (e.g., see Example 3).

In some embodiments, there are several advantages to not using a guide molecule in the ANAGO induced precise genomic sequence editing (AISE) technology disclosed herein. First, the percentages of on-target HDR precise editing using the AISE in the presence of a guide molecule and a donor molecule is lower than that of the AISE in the presence of a donor molecule alone without a guide molecule. The difference in the percentages of on-target HDR precise editing between the AISE with a guide molecule and the AISE without a guide molecule can be significant. In some embodiments, the percentages of on-target HDR precise editing using AISE in the presence of a guide molecule and a donor molecule is about 1-10 times, about 1-6 times, about 1-5 times, about 1-4 times, about 1-3 times, about 1 times, about 2 times, about 3 times, about 4 times, about 5 times, about 6 times, about 7 times, about 8 times, about 9 times, about 10 times, about 1%-40%, about 1%-30%, about 1%-20%, about 1%-10%, about 10%-20%, about 20%-30%, about 1%-5%, about 1%-4%, about 1%-3%, about 1%-2%, about 2%-3%, about 3%-4%, about 4%-5%, about 5%-6%, about 6%-7%, about 7%-8%, about 8%-9%, about 9%-10%, about 10%-15%, about 15%-20%, about 20%-25%, about 25%-30%, about 5%-10%, about 1%, about 2%, about 3%, about 3.2%, about 5%, about 10%, about 15%, about 15.3%, or about 20% lower than that of the AISE in the presence of a donor molecule alone without a guide molecule, or any percentage bounded by any of the above values. It is possible that the guide molecule in the AISE described herein may compete with the donor molecule during the sequence editing. Therefore, not only is a guide molecule not required for ANAGO induced precise genomic sequence editing (AISE), but there may also be an advantage in not using a guide molecule in the AISE described herein in achieving precise genomic editing with high yield/percentage.

Furthermore, to prepare a guide molecule that is specific to each given target sequence and to incorporate the guide molecule in AISE requires additional material, synthesis time, purification time, and steps. This may be more time consuming and inconvenient, which would certainly increase overall treatment cost and prolong the overall treatment time when AISE is applied in gene therapy for treating gene related various diseases, disorders, or conditions.

A donor sequence (a donor fragment) is a nucleic acid comprising three parts, a 5′ flanking sequence, a desired sequence, and a 3′ flanking sequence. In some embodiments, a donor sequence comprises RNA. In certain embodiments, a donor sequence comprises DNA. In some embodiments, a donor sequence is single stranded. In some embodiments, a donor sequence is double stranded. A donor sequence can be short or long in length.

The 5′-flanking sequence and the 3′-flanking sequences are different sequences. In some embodiments, the 5′-flanking sequence and the 3′-flanking sequences do not share more than 10% identity. In some embodiments, the 5′-flanking sequence and the 3′-flanking sequences are located on opposite sides of the desired sequence. Each of 5′ flanking sequence and/or each of 3′ flanking sequence of a donor sequence, in certain embodiments, is independently about 500 nucleotides (nt) or base pairs (bp) in length, or longer, about 100 nt or bp or longer, 100-200 nt or bp, 10-200 nt or bp, 10-100 nt or bp, 10-75 nt or bp, 75-100 nt or bp, 10-50 nt or bp, 19-50 nt or bp, 19-30 nt or bp, 16-50 nt or bp, 16-30 nt or bp, 10-25 nt or bp, 20-25 nt or bp, 10-20 nt or bp, 20-30 nt or bp, 30-40 nt or bp, 40-50 nt or bp, 50-60 nt or bp, 60-70 nt or bp, 70-80 nt or bp, 80-90 nt or bp, 90-100 nt or bp, 10-15 nt or bp, 15-25 nt or bp, 25-35 nt or bp, 35-45 nt or bp, 45-55 nt or bp, 55-65 nt or bp, 65-75 nt or bp, 75-85 nt or bp, 85-95 nt or bp, 95-100 nt or bp, 16-20 nt or bp, 20-25 nt or bp, 25-30 nt or bp, 30-35 nt or bp, 35-40 nt or bp, 40-45 nt or bp, 45-50 nt or bp, 50-55 nt or bp, 55-60 nt or bp, 60-65 nt or bp, 65-70 nt or bp, 70-75 nt or bp, 75-80 nt or bp, 80-85 nt or bp, 85-90 nt or bp, 90-95 nt or bp, 19-50 nt or bp, 19-30 nt or bp, 16-20 nt or bp, 10 nt or bp, 11 nt or bp, 12 nt or bp, 13 nt or bp, 14 nt or bp, 15 nt or bp, 16 nt or bp, 17 nt or bp, 18 nt or bp, 19 nt or bp, 20 nt or bp, 21 nt or bp, 22 nt or bp, 23 nt or bp, 24 nt or bp, 25 nt or bp, 26 nt or bp, 27 nt or bp, 28 nt or bp, 29 nt or bp, 30 nt or bp, 31 nt or bp, 32 nt or bp, 33 nt or bp, 34 nt or bp, 35 nt or bp, 36 nt or bp, 37 nt or bp, 38 nt or bp, 39 nt or bp, 40 nt or bp, 41 nt or bp, 42 nt or bp, 43 nt or bp, 44 nt or bp, 45 nt or bp, 46 nt or bp, 47 nt or bp, 48 nt or bp, 49 nt or bp, 50 nt or bp, 51 nt or bp, 52 nt or bp, 53 nt or bp, 54 nt or bp, 55 nt or bp, 56 nt or bp, 57 nt or bp, 58 nt or bp, 59 nt or bp, 60 nt or bp, 61 nt or bp, 62 nt or bp, 63 nt or bp, 64 nt or bp, 65 nt or bp, 66 nt or bp, 67 nt or bp, 68 nt or bp, 69 nt or bp, 70 nt or bp, 71 nt or bp, 72 nt or bp, 73 nt or bp, 74 nt or bp, 75 nt or bp, 76 nt or bp, 77 nt or bp, 78 nt or bp, 79 nt or bp, 80 nt or bp, 81 nt or bp, 82 nt or bp, 83 nt or bp, 84 nt or bp, 85 nt or bp, 86 nt or bp, 87 nt or bp, 88 nt or bp, 89 nt or bp, 90 nt or bp, 91 nt or bp, 92 nt or bp, 93 nt or bp, 94 nt or bp, 95 nt or bp, 96 nt or bp, 97 nt or bp, 98 nt or bp, 99 nt or bp, or 100 nt or bp in length, or any number in a range bounded by any of the above values of nucleotides (nt) or base pairs (bp) in length. Each of the 5′-flanking sequence and each of the 3′-flanking sequence independently comprise at least 10 nucleotides that are identical to the target sequence.

A “desired sequence” in a donor nucleic acid refers to a nucleic acid that is to be inserted into a target sequence induced by an ANAGO and/or by a method described herein. The term “desired sequence” is used synonymously with the terms “desired nucleic acid” and “desired nucleic acid sequence”. For purposes of clarity, a “desired sequence” may sometimes be referred to as an “insert sequence”. In some embodiments, a desired sequence comprises RNA. In certain embodiments, a desired sequence comprises DNA. A desired sequence can be any suitable sequence of any suitable length. In some embodiments, a desired sequence is 1-20,000 nt, 1-10,000 nt, 10,000-20,000 nt, 10,000-15,000 nt, 15,000-20,000 nt, 1-5,000 nt, 1-2500 nt, 2500-5000 nt, 1000-2000 nt, 2000-3000 nt, 3000-4000 nt, 4000-5000 nt, 1-1000 nt, 1-500 nt, 10-5,000 nt, 10-1000 nt, 10-500 nt, 1-100 nt, 100-200 nt, 200-300 nt, 300-400 nt, 400-500 nt, 500-600 nt, 600-700 nt, 700-800 nt, 800-900 nt, 900-1000 nt, 200-400 nt, 400-600 nt, 600-800 nt, 1-50 nt, 50-100 nt, 60-80 nt, 80-100 nt, 18,000 nt, 700 nt, 89 nt, 72 nt, or 70 nucleotides long, or any number in a range bounded by any of the above values of nucleotides in length. In some embodiments, a donor sequence comprises a 5′ flanking sequence and a 3′ flanking sequence that are each, independently, 80% to 100%, 80%-90%, 90%-100%, 80%-85%, 85%-90%, 90%-95%, 95%-100%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a target site.

Accordingly, in certain embodiments, presented herein is a method of editing a genome of an organism or cell. In certain embodiments, the organism is a subject. In some embodiments, the subject is a human. A cell may be any suitable cell, non-limiting examples of which include a prokaryotic cell, plant cell, eukaryotic cell, mammalian cell or human cell. In certain embodiments, a method of editing a genome comprises removal of a target sequence from a genome, disruption of a target sequence within a genome and/or insertion of a desired sequence into a genome. A desired sequence can be any suitable nucleic acid sequence non-limiting examples of which include a sequence of a heterologous nucleic acid (e.g., from a different species), a modified heterologous nucleic acid, a homologous nucleic acid (e.g., from the same species), a synthetic nucleic acid, a gene or portion thereof (e.g., intron, exon, regulatory sequences, etc.), a modified gene, a marker, a toxin, a single nucleic acid, two or more nucleic acids, the like or a combinations thereof. In some embodiments, a desired nucleic acid encodes a chimeric antigen receptor (CAR).

A desired nucleic acid (desired sequence) or gene can be any suitable mammalian gene, portion thereof, or modified form thereof, non-limiting examples of which include human genes A2M, AACS, AARSD1, ABCA10, ABCA12, ABCA3, ABCA8, ABCA9, ABCB1, ABCB10, ABC84, ABCC11, ABCC12, ABCC6, ABCD1, ABCE1, ABCF1, ABCF2, ABT1, ACAA2, ACCSL, ACER2, ACO2, ACOT1, ACOT4, ACOT7, ACP1, ACR, ACRC, ACSBG2, ACSM1, ACSM2A, ACSM2B, ACSM4, ACSM5, ACTA1, ACTA2, ACTB, ACTG1, ACTG2, ACTN1, ACTN4, ACTR1A, ACTR2, ACTR3, ACTR3C, ACTRT1, ADAD1, ADAL, ADAM18, ADAM20, ADAM21, ADAM32, ADAMTS7, ADAMTSL2, ADAT2, ADCY5, ADCY6, ADCY7, ADGB, ADH1A, ADH1B, ADH1C, ADH5, ADORA2B, ADRBK2, ADSS, AFF3, AFF4, AFG3L2, AGAP1, AGAP10, AGAP11, AGAP4, AGAP5, AGAP6, AGAP7, AGAP8, AGAP9, AGER, AGGF1, AGK, AGPAT1, AGPAT6, AHCTF1, AHCY, AHNAK2, AHRR, AIDA, AIF1, AIM1L, AIMP2, AK2, AK3, AK4, AKAP13, AKAP17A, AKIP1, AKIRIN1, AKIRIN2, AKR1B1, AKR1B10, AKR1B15, AKR1C1, AKR1C2, AKR1C3, AKR1C4, AKR7A2, AKR7A3, AKTIP, ALDH3B1, ALDH3B2, ALDH7A1, ALDOA, ALG1, ALG10, ALG10B, ALG1L, ALG1L2, ALG3, ALKBH8, ALMS1, ALOX15, ALOX15B, ALOXE3, ALPI, ALPP, ALPPL2, ALYREF, AMD1, AMELX, AMELY, AMMECR1L, AMY1A, AMY1B, AMY1C, AMY2A, AMY2B, AMZ2, ANAPC1, ANAPC10, ANAPC15, ANKRD11, ANKRD18A, ANKRD18B, ANKRD20A1, ANKRD20A19P, ANKRD20A2, ANKRD20A3, ANKRD20A4, ANKRD30A, ANKRD30B, ANKRD36, ANKRD36B, ANKRD49, ANKS1B, ANO10, ANP32A, ANP32B, ANXA2, ANXA2R, ANXA8, ANXA8L1, ANXA8L2, AOC2, AOC3, AP1B1, AP1S2, AP2A1, AP2A2, AP2B1, AP2S1, AP3M2, AP3S1, AP4S1, APBA2, APBB1IP, APH1B, API5, APIP, APOBEC3A, APOBEC3B, APOBEC3C, APOBEC3D, APOBEC3F, APOBEC3G, APOC1, APOL1, APOL2, APOL4, APOM, APOOL, AQP10, AQP12A, AQP12B, AQP7, AREG, AREGB, ARF1, ARF4, ARF6, ARGFX, ARHGAP11A, ARHGAP11B, ARHGAP20, ARHGAP21, ARHGAP23, ARHGAP27, ARHGAP42, ARHGAP5, ARHGAP8, ARHGEF35, ARHGEF5, ARID2, ARID3B, ARIH2, ARL14EP, ARL16, ARL17A, ARL17M, ARL2BP, ARL4A, ARL5A, ARL6IP1, ARL6IP6, ARL8B, ARMC1, ARMC10, ARMC4, ARMC8, ARMCX6, ARPC1A, ARPC2, ARPC3, ARPP19, ARSD, ARSE, ARSF, ART3, ASAH2, ASAH2B, ASB9, ASL, ASMT, ASMTL, ASNS, ASS1, ATAD1, ATAD3A, ATAD3B, ATAD3C, ATAT1, ATF4, ATF6B, ATF7IP2, ATG4A, ATM, ATMIN, ATP13A4, ATP13A5, ATP1A2, ATP1A4, ATP1B1, ATP1B3, ATP2B2, ATP2B3, ATP5A1, ATP5C1, ATP5F1, ATP5G1, ATP5G2, ATP5G3, ATP5H, ATP5J, ATP5J2, ATP5J2-PTCD1, ATP5O, ATP6AP2, ATP6VOC, ATP6V1E1, ATP6V1F, ATP6V1G1, ATP6V1G2, ATP7B, ATP8A2, ATP9B, ATXN1L, ATXN2L, ATXN7L3, AURKA, AURKAIP1, AVP, AZGP1, AZI2, B3GALNT1, B3GALT4, B3GAT3, B3GNT2, BAG4, BAG6, BAGE2, BAK1, BANF1, BANP, BCAP31, BCAR1, BCAS2, BCL2A1, BCL2L12, BCL2L2-PABPN1, BCLAF1, BCOR, BCR, BDH2, BDP1, BEND3, BET1, BEX1, BHLHB9, BHLHE22, BHLHE23, BHMT, BHMT2, BIN2, BIRC2, BIRC3, BLOC1S6, BLZF1, BMP2K, BMP8A, BMP8B, BMPR1A, BMS1, BNIP3, BOD1, BOD1L2, BOLA2, BOLA2B, BOLAS, BOP1, BPTF, BPY2, BPY2B, BPY2C, BRAF, BRCA1, BRCC3, BRD2, BRD7, BRDT, BRI3, BRK1, BRPF1, BRPF3, BRWD1, BTBD10, BTBD6, BTBD7, BTF3, BTF3L4, BTG1, BTN2A1, BTN2A2, BTN3A1, BTN3A2, BTN3A3, BTNL2, BTNL3, BTNL8, BUB3, BZW1, C10orf129, C10orf88, C11orf48, C11orf58, C11orf74, C11orf75, C12orf29, C12orf42, C12orf49, C12orf71, C12orf76, C14orf119, C14orf166, C14orf178, C15orf39, C15orf40, C15orf43, C16orf52, C16orf88, C17orf51, C17orf58, C17orf61, C17orf89, C17orf98, C18orf21, C18orf25, C1D, C1GALT1, C1QBP, C1QL1, C1QL4, C1QTNF9, C1QTNF9B, C1QTNF9B-AS1, C1orf100, C1orf106, C1orf114, C2, C22orf42, C22orf43, C2CD4A, C2orf16, C2orf27A, C2orf27B, C2orf69, C2orf78, C2orf81, C4A, C4B, C4BPA, C4orf27, C4orf34, C4orf46, C5orf15, C5orf43, C5orf52, C5orf60, C5orf63, C6orf10, C6orf106, C6orf136, C6orf15, C6orf203, C6orf25, C6orf47, C6orf48, C7orf63, C7orf73, C8orf46, C9orf123, C9orf129, C9orf172, C9orf57, C9orf69, C9orf78, CA14, CA15P3, CA5A, CA5B, CABYR, CACNA1C, CACNA1G, CACNA1H, CACNA1I, CACYBP, CALCA, CALCB, CALM1, CALM2, CAMSAP1, CAP1, CAPN8, CAPZA1, CAPZA2, CARD16, CARD17, CASC4, CASP1, CASP3, CASP4, CASP5, CATSPER2, CBR1, CBR3, CBWD1, CBWD2, CBWD3, CBWD5, CBWD6, CBWD7, CBX1, CBX3, CCDC101, CCDC111, CCDC121, CCDC127, CCDC14, CCDC144A, CCDC144NL, CCDC146, CCDC150, CCDC174, CCDC25, CCDCl58, CCDC7, CCDC74A, CCDC74B, CCDC75, CCDC86, CCHCR1, CCL15, CCL23, CCL3, CCL3L1, CCL3L3, CCL4, CCL4L1, CCL4L2, CCNB1IP1, CCNB2, CCND2, CCNG1, CCNJ, CCNT2, CCNYL1, CCR2, CCR5, CCRL1, CCRN4L, CCT4, CCT5, CCT6A, CCT7, CCT8, CCT8L2, CCZ1, CCZ1B, CD177, CD1A, CD1B, CD1C, CD1D, CD1E, CD200R1, CD200R1L, CD209, CD276, CD2BP2, CD300A, CD300C, CD300LD, CD300LF, CD33, CD46, CD83, CD8B, CD97, CD99, CDC14B, CDC20, CDC26, CDC27, CDC37, CDC42, CDC42EP3, CDCA4, CDCA7L, CDH12, CDK11A, CDK11B, CDK2AP2, CDK5RAP3, CDK7, CDK8, CDKN2A, CDKN2AIPNL, CDKN2B, CDON, CDPF1, CDRT1, CDRT15, CDRT15L2, CDSN, CDV3, CDY1, CDY2A, CDY2B, CEACAM1, CEACAM18, CEACAM21, CEACAM3, CEACAM4, CEACAM5, CEACAM6, CEACAM7, CEACAM8, CEL, CELA2A, CELA2B, CELA3A, CELA3B, CELSR1, CEND1, CENPC1, CENPI, CENPJ, CENPO, CEP170, CEP19, CEP192, CEP290, CEP57L1, CES1, CES2, CES5A, CFB, CFC1, CFC1B, CFH, CFHR1, CFHR2, CFHR3, CFHR4, CFHR5, CFL1, CFTR, CGB, CGB1, CGB2, CGB5, CGB7, CGB8, CHAF1B, CHCHD10, CHCHD2, CHCHD3, CHCHD4, CHD2, CHEK2, CHIA, CHMP4B, CHMP5, CHORDC1, CHP1, CHRAC1, CHRFAM7A, CHRNA2, CHRNA4, CHRNB2, CHRNB4, CHRNE, CHST5, CHST6, CHSY1, CHTF8, CIAPIN1, CIC, CIDEC, CIR1, CISD1, CISD2, CKAP2, CKMT1A, CKMT1B, CKS2, CLC, CLCN3, CLCNKA, CLCNKB, CLDN22, CLDN24, CLDN3, CLDN4, CLDN6, CLDN7, CLEC17A, CLEC18A, CLEC18B, CLEC18C, CLEC1A, CLEC1B, CLEC4G, CLEC4M, CLIC1, CLIC4, CLK2, CLK3, CLK4, CLNS1A, CMPK1, CMYA5, CNEP1R1, CNN2, CNN3, CNNM3, CNNM4, CNOT6L, CNOT7, CNTNAP3, CNTNAP3B, CNTNAP4, COA5, COBL, COIL, COL11A2, COL12A1, COL19A1, COL25A1, COL28A1, COL4A5, COL6A5, COL6A6, COMMD4, COMMD5, COPR5, COPRS, COPS5, COQ10B, CORO1A, COX10, COX17, COX20, COX5A, COX6A1, COX6B1, COX7B, COX7C, COX8C, CP, CPAMD8, CPD, CPEB1, CPSF6, CR1, CR1L, CRADD, CR83, CRCP, CREBBP, CRHR1, CRLF2, CRLF3, CRNN, CROCC, CRTC1, CRYBB2, CRYGB, CRYGC, CRYGD, CS, CSAG1, CSAG2, CSAG3, CSDA, CSDE1, CSF2RA, CSF2RB, CSGALNACT2, CSH1, CSH2, CSHL1, CSNK1A1, CSNK1D, CSNK1E, CSNK1G2, CSNK2A1, CSNK2B, CSPG4, CSRP2, CST1, CST2, CST3, CST4, CST5, CST9, CT45A1, CT45A2, CT45A3, CT45A4, CT45A5, CT45A6, CT47A1, CT47A10, CT47A11, CT47A12, CT47A2, CT47A3, CT47A4, CT47A5, CT47A6, CT47A7, CT47A8, CT47A9, CT47B1, CTAG1A, CTAG1B, CTAG2, CTAGE1, CTAGE5, CTAGE6P, CTAGE9, CT8P2, CTDNEP1, CTDSP2, CTDSPL2, CTLA4, CTNNA1, CTNND1, CTRB1, CTRB2, CTSL1, CTU1, CUBN, CUL1, CUL7, CUL9, CUTA, CUX1, CXADR, CXCL1, CXCL17, CXCL2, CXCL3, CXCL5, CXCL6, CXCR1, CXCR2, CXorf40A, CXorf40B, CXorf48, CXorf49, CXorf49B, CXorf56, CXorf61, CYB5A, CYC5, CYP11B1, CYP11B2, CYP1A1, CYP1A2, CYP21A2, CYP2A13, CYP2A6, CYP2A7, CYP2B6, CYP2C18, CYP2C19, CYP2C8, CYP2C9, CYP2D6, CYP2F1, CYP3A4, CYP3A43, CYP3A5, CYP3A7, CYP3A7-CYP3AP1, CYP46A1, CYP4A11, CYP4A22, CYP4F11, CYP4F12, CYP4F2, CYP4F3, CYP4F8, CYP4Z1, CYP51A1, CYorf17, DAP3, DAPK1, DAXX, DAZ1, DAZ2, DAZ3, DAZ4, DAZAP2, DAZL, DBF4, DCAF12L1, DCAF12L2, DCAF13, DCAF4, DCAF4L1, DCAF4L2, DCAF6, DCAF8L1, DCAF8L2, DCLRE1C, DCTN6, DCUN1D1, DCUN1D3, DDA1, DDAH2, DDB2, DDR1, DDT, DDTL, DDX10, DDX11, DDX18, DDX19A, DDX19B, DDX23, DDX26B, DDX39B, DDX3X, DDX3Y, DDX50, DDX55, DDX56, DDX6, DDX60, DDX60L, DEF8, DEFB103A, DEFB103B, DEFB104A, DEFB104B, DEFB105A, DEFB105B, DEFB106A, DEFB106B, DEFB107A, DEFB107B, DEFB108B, DEFB130, DEFB131, DEFB4A, DEFB4B, DENND1C, DENR, DEPDC1, DERL2, DESI2, DEXI, DGCR6, DGCR6L, DGKZ, DHFR, DHFRL1, DHRS2, DHRS4, DHRS4L1, DHRS4L2, DHRSX, DHX16, DHX29, DHX34, DHX40, DICER1, DIMT1, DIS3L2, DKKL1, DLEC1, DLST, DMBT1, DMRTC1, DMRTC1B, DNAH11, DNAJA1, DNAJA2, DNAJB1, DNAJB14, DNAJB3, DNAJB6, DNAJC1, DNAJC19, DNAJC24, DNAJC25-GNG10, DNAJC5, DNAJC7, DNAJC8, DNAJC9, DND1, DNM1, DOCK1, DOCK11, DOCK9, DOK1, DOM3Z, DONSON, DPCR1, DPEP2, DPEP3, DPF2, DPH3, DPM3, DPP3, DPPA2, DPPA3, DPPA4, DPPA5, DPRX, DPY19L1, DPY19L2, DPY19L3, DPY19L4, DPY30, DRAXIN, DRD5, DRG1, DSC2, DSC3, DSE, DSTN, DTD2, DTWD1, DTWD2, DTX2, DUOX1, DUOX2, DUSP12, DUSP5, DUSP8, DUT, DUXA, DYNC1I2, DYNC1LI1, DYNLT1, DYNLT3, E2F3, EBLN1, EBLN2, EBPL, ECEL1, EDDM3A, EDDM3B, EED, EEF1A1, EEF1B2, EEF1D, EEF1E1, EEF1G, EFCAB3, EFEMP1, EFTUD1, EGFL8, EGLN1, EHD1, EHD3, EHMT2, EI24, EIF1, EIF1AX, EIF2A, EIF2C1, EIF2C3, EIF2S2, EIF2S3, EIF3A, EIF3C, EIF3CL, EIF3E, EIF3F, EIF3J, EIF3L, EIF3M, EIF4A1, EIF4A2, EIF4B, EIF4E, EIF4E2, EIF4EBP1, EIF4EBP2, EIF4H, EIF5, EIF5A, EIF5A2, EIF5AL1, ELF2, ELK1, ELL2, ELMO2, EMB, EMC3, EMR1, EMR2, EMR3, ENAH, ENDOD1, ENO1, ENO3, ENPEP, ENPP7, ENSA, EP300, EP400, EPB41L4B, EPB41L5, EPCAM, EPHA2, EPHB2, EPHB3, EPN2, EPN3, EPPK1, EPX, ERCC3, ERF, ERP29, ERP44, ERVV-1, ERVV-2, ESCO1, ESF1, ESPL1, ESPN, ESRRA, ETF1, ETS2, ETV3, ETV3L, EVA1C, EVPL, EVPLL, EWSR1, EXOC5, EXOC8, EXOG, EXOSC3, EXOSC6, EXTL2, EYS, EZR, F5, F8A1, F8A2, F8A3, FABP3, FABP5, FAF2, FAHD1, FAHD2A, FAHD2B, FAM103A1, FAM104B, FAM108A1, FAM108C1, FAM111B, FAM115A, FAM115C, FAM120A, FAM120B, FAM127A, FAM127B, FAM127C, FAM131C, FAM133B, FAM136A, FAM149B1, FAM151A, FAM153A, FAM153B, FAM154B, FAM156A, FAM156B, FAM157A, FAM157B, FAM163B, FAM165B, FAM175A, FAM177A1, FAM185A, FAM186A, FAM18B1, FAM18B2, FAM190B, FAM192A, FAM197Y1, FAM197Y3, FAM197Y4, FAM197Y6, FAM197Y7, FAM197Y8, FAM197Y9, FAM203A, FAM203B, FAM204A, FAM205A, FAM206A, FAM207A, FAM209A, FAM209B, FAM20B, FAM210B, FAM213A, FAM214B, FAM218A, FAM21A, FAM21B, FAM21C, FAM220A, FAM22A, FAM22D, FAM22F, FAM22G, FAM25A, FAM25B, FAM25C, FAM25G, FAM27E4P, FAM32A, FAM35A, FAM3C, FAM45A, FAM47A, FAM47B, FAM47C, FAM47E-STBD1, FAM58A, FAM60A, FAM64A, FAM72A, FAM72B, FAM72D, FAM76A, FAM83G, FAM86A, FAM86B2, FAM86C1, FAM89B, FAM8A1, FAM90A1, FAM91A1, FAM92A1, FAM96A, FAM98B, FAM9A, FAM9B, FAM9C, FANCD2, FANK1, FAR1, FAR2, FARP1, FARSB, FASN, FASTKD1, FAT1, FAU, FBLIM1, FBP2, FBRSL1, FBXL12, FBXO25, FBXO3, FBXO36, FBXO44, FBXO6, FBXW10, FBXW11, FBXW2, FBXW4, FCF1, FCGBP, FCGR1A, FCGR2A, FCGR2B, FCGR3A, FCGR3B, FCN1, FCN2, FCRL1, FCRL2, FCRL3, FCRL4, FCRL5, FCRL6, FDPS, FDX1, FEM1A, FEN1, FER, FFAR3, FGD5, FGF7, FGFR1OP2, FH, FHL1, FIGLA, FKBP1A, FKBP4, FKBP6, FKBP8, FKBP9, FKBPL, FLG, FLG2, FLI1, FLI44635, FLNA, FLNB, FLNC, FLOT1, FLT1, FLYWCH1, FMN2, FN3K, FOLH1, FOLH1B, FOLR1, FOLR2, FOLR3, FOSL1, FOXA1, FOXA2, FOXA3, FOXD1, FOXD2, FOXD3, FOXD4L2, FOXD4L3, FOXD4L6, FOXF1, FOXF2, FOXH1, FOXN3, FOXO1, FOXO3, FPR2, FPR3, FRAT2, FREM2, FRG1, FRG2, FRG2B, FRG2C, FRMD6, FRMD7, FRMD8, FRMPD2, FSCN1, FSIP2, FTH1, FTHL17, FTL, FTO, FUNDC1, FUNDC2, FUT2, FUT3, FUT5, FUT6, FXN, FXR1, FZD2, FZD5, FZD8, G2E3, G3BP1, GABA RAP, GABARAPL1, GABBR1, GABPA, GABRP, GABRR1, GABRR2, GAGE1, GAGE10, GAGE12C, GAGE12D, GAGE12E, GAGE12F, GAGE12G, GAGE12H, GAGE12I, GAGE12J, GAGE13, GAGE2A, GAGE2B, GAGE2C, GAGE2D, GAGE2E, GAPDH, GAR1, GATS, GATSL1, GATSL2, GBA, GBP1, GBP2, GBP3, GBP4, GBP5, GBP6, GBP7, GCAT, GCDH, GCNT1, GCOM1, GCSH, GDI2, GEMIN7, GEMIN8, GFRA2, GGCT, GGT1, GGT2, GGT5, GGTLC1, GGTLC2, GH1, GH2, GINS2, GJA1, GJC3, GK, GK2, GLB1L2, GLB1L3, GLDC, GLOD4, GLRA1, GLRA4, GLRX, GLRX3, GLRX5, GLTP, GLTSCR2, GLUD1, GLUL, GLYATL1, GLYATL2, GLYR1, GM2A, GMCL1, GMFB, GMPS, GNA11, GNAQ, GNAT2, GNG10, GNG5, GNGT1, GNL1, GNL3, GNL3L, GNPNAT1, GOLGA2, GOLGA4, GOLGA5, GOLGA6A, GOLGA6B, GOLGA6C, GOLGA6D, GOLGA6L1, GOLGA6L10, GOLGA6L2, GOLGA6L3, GOLGA6L4, GOLGA6L6, GOLGA6L9, GOLGA7, GOLGA8H, GOLGA8J, GOLGA8K, GOLGA8O, GON4L, GOSR1, GOSR2, GOT2, GPAA1, GPANK1, GPAT2, GPATCH8, GPC5, GPCPD1, GPD2, GPHN, GPN1, GPR116, GPR125, GPR143, GPR32, GPR89A, GPR89B, GPR89C, GPS2, GPSM3, GPX1, GPX5, GPX6, GRAP, GRAPL, GRIA2, GRIA3, GRIA4, GRK6, GRM5, GRM8, GRPEL2, GSPT1, GSTA1, GSTA2, GSTA3, GSTA5, GSTM1, GSTM2, GSTM4, GSTM5, GSTO1, GSTT1, GSTT2, GSTT2B, GTF2A1L, GTF2H1, GTF2H2, GTF2H2C, GTF2H4, GTF2I, GTF2IRD1, GTF2IRD2, GTF2IRD2B, GTF3C6, GTPBP6, GUSB, GXYLT1, GYG1, GYG2, GYPA, GYPB, GYPE, GZMB, GZMH, H1FOO, H2AFB1, H2AFB2, H2AFB3, H2AFV, H2AFX, H2AFZ, H2BFM, H2BFWT, H3F3A, H3F3B, H3F3C, HADHA, HADHB, HARS, HARS2, HAS3, HAUS1, HAUS4, HAUS6, HAVCR1, HAX1, HBA1, HBA2, HBB, HBD, HBG1, HBG2, HBS1L, HBZ, HCAR2, HCAR3, HCN2, HCN3, HCN4, HDAC1, HDGF, HDHD1, HEATR7A, HECTD4, HERC2, HIATL1, HIBCH, HIC1, HIC2, HIGD1A, HIGD2A, HINT1, HIST1H1B, HIST1H1C, HIST1H1D, HIST1H2AA, HIST1H2AB, HIST1H2AC, HIST1H2AD, HIST1H2AE, HIST1H2AG, HIST1H2AH, HIST1H2AI, HIST1H2AL, HIST1H2BB, HIST1H2BD, HIST1H2BE, HIST1H2BF, HIST1H2BH, HIST1H2BI, HIST1H2BK, HIST1H2BM, HIST1H2BN, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, HIST2H2AA3, HIST2H2AB, HIST2H2AC, HIST2H2BE, HIST2H2BF, HIST2H3A, HIST2H3D, HIST2H4A, HIST2H4B, HIST3H2BB, HIST3H3, HIST4H4, HK2, HLA-A, HLA-B, HLA-C, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB5, HLA-E, HLA-F, HLA-G, HMGA1, HMGB1, HMGB2, HMGB3, HMGCS1, HMGN1, HMGN2, HMGN3, HMGN4, HMX1, HMX3, HNRNPA1, HNRNPA3, HNRNPAB, HNRNPC, HNRNPCL1, HNRNPD, HNRNPF, HNRNPH1, HNRNPH2, HNRNPH3, HNRNPK, HNRNPL, HNRNPM, HNRNPR, HNRNPU, HNRPDL, HOMER2, HORMAD1, HOXA2, HOXA3, HOXA6, HOXA7, HOXB2, HOXB3, HOXB6, HOXB7, HOXD3, HP, HPR, HPS1, HRG, HS3ST3A1, HS3ST3B1, HS6ST1, HSD17B1, HSD17B12, HSD17B4, HSD17B6, HSD17B7, HSD17B8, HSD3B1, HSD3B2, HSF2, HSFX1, HSFX2, HSP90AA1, HSP90AB1, HSP90B1, HSPA14, HSPA1A, HSPA1B, HSPA1L, HSPA2, HSPA5, HSPA6, HSPA8, HSPA9, HSPB1, HSPD1, HSPE1, HSPE1-MOB4, HSPG2, HTN1, HTN3, HTR3C, HTR3D, HTR3E, HTR7, HYDIN, HYPK, IARS, ID2, IDH1, IDI1, IDS, IER3, IFI16, IFIH1, IFIT1, IFIT1B, IFIT2, IFIT3, IFITM3, IFNA1, IFNA10, IFNA14, IFNA16, IFNA17, IFNA2, IFNA21, IFNA4, IFNA5, IFNA6, IFNA7, IFNA8, IFT122, IFT80, IGBP1, IGF2BP2, IGF2BP3, IGFL1, IGFL2, IGFN1, IGLL1, IGLL5, IGLON5, IGSF3, IHH, IK, IKBKG, IL17RE, IL18, IL28A, IL28B, IL29, IL32, IL3RA, IL6ST, IL9R, IMMP1L, IMMT, IMPA1, IMPACT, IMPDH1, ING5, INIP, INTS4, INTS6, IPMK, IPO7, IPPK, IQCB1, IREB2, IRX2, IRX3, IRX4, IRX5, IRX6, ISCA1, ISCA2, ISG20L2, ISL1, ISL2, IST1, ISY1-RAB43, ITFG2, ITGAD, ITGAM, ITGAX, ITGB1, ITGB6, ITIH6, ITLN1, ITLN2, ITSN1, KAL1, KANK1, KANSL1, KARS, KAT7, KATNBL1, KBTBD6, KBTBD7, KCNA1, KCNA5, KCNA6, KCNC1, KCNC2, KCNC3, KCNH2, KCNH6, KCNJ12, KCNJ4, KCNMB3, KCTD1, KCTD5, KCTD9, KDELC1, KDM5C, KDM5D, KDM6A, KHDC1, KHDC1L, KHSRP, KIAA0020, KIAA0146, KIAA0494, KIAA0754, KIAA0895L, KIAA1143, KIAA1191, KIAA1328, KIAA1377, KIAA1462, KIAA1549L, KIAA1551, KIAA1586, KIAA1644, KIAA1671, KIAA2013, KIF1C, KIF27, KIF4A, KIF4B, KIFC1, KIR2DL1, KIR2DL3, KIR2DL4, KIR2DS4, KIR3DL1, KIR3DL2, KIR3DL3, KLF17, KLF3, KLF4, KLF7, KLF8, KLHL12, KLHL13, KLHL15, KLHL2, KLHL5, KLHL9, KLK2, KLK3, KLRC1, KLRC2, KLRC3, KLRC4, KNTC1, KPNA2, KPNA4, KPNA7, KPNB1, KRAS, KRT13, KRT14, KRT15, KRT16, KRT17, KRT18, KRT19, KRT25, KRT27, KRT28, KRT3, KRT31, KRT32, KRT33A, KRT33B, KRT34, KRT35, KRT36, KRT37, KRT38, KRT4, KRT5, KRT6A, KRT6B, KRT6C, KRT71, KRT72, KRT73, KRT74, KRT75, KRT76, KRT8, KRT80, KRT81, KRT82, KRT83, KRT85, KRT86, KRTAP1-1, KRTAP1-3, KRTAP1-5, KRTAP10-10, KRTAP10-11, KRTAP10-12, KRTAP10-2, KRTAP10-3, KRTAP10-4, KRTAP10-7, KRTAP10-9, KRTAP12-1, KRTAP12-2, KRTAP12-3, KRTAP13-1, KRTAP13-2, KRTAP13-3, KRTAP13-4, KRTAP19-1, KRTAP19-5, KRTAP2-1, KRTAP2-2, KRTAP2-3, KRTAP2-4, KRTAP21-1, KRTAP21-2, KRTAP23-1, KRTAP3-2, KRTAP3-3, KRTAP4-12, KRTAP4-4, KRTAP4-6, KRTAP4-7, KRTAP4-9, KRTAP5-1, KRTAP5-10, KRTAP5-3, KRTAP5-4, KRTAP5-6, KRTAP5-8, KRTAP5-9, KRTAP6-1, KRTAP6-2, KRTAP6-3, KRTAP9-2, KRTAP9-3, KRTAP9-6, KRTAP9-8, KRTAP9-9, L1TD1, LAGE3, LAIR1, LAIR2, LAMTOR3, LANCL3, LAP3, LAPTM4B, LARP1, LARP1B, LARP4, LARP7, LCE1A, LCE1B, LCE1C, LCE1D, LCE1E, LCE1F, LCE2A, LCE2B, LCE2C, LCE2D, LCE3C, LCE3D, LCE3E, LCMT1, LCN1, LDHA, LDHAL6B, LDHB, LEFTY1, LEFTY2, LETM1, LGALS13, LGALS14, LGALS16, LGALS7, LGALS7B, LGALS9, LGALS9B, LGALS9C, LGMN, LGR6, LHB, LILRA1, LILRA2, LILRA3, LILRA4, LILRA5, LILRA6, LILRB1, LILRB2, LILRB3, LILRB4, LILRB5, LIMK2, LIMS1, LIN28A, LIN28B, LIN54, LLPH, LMLN, LNX1, LOC100129083, LOC100129216, LOC100129307, LOC100129636, LOC100130539, LOC100131107, LOC100131608, LOC100132154, LOC100132202, LOC100132247, LOC100132705, LOC100132858, LOC100132859, LOC100132900, LOC100133251, LOC100133267, LOC100133301, LOC100286914, LOC100287294, LOC100287368, LOC100287633, LOC100287852, LOC100288332, LOC100288646, LOC100288807, LOC100289151, LOC100289375, LOC100289561, LOC100505679, LOC100505767, LOC100505781, LOC100506248, LOC100506533, LOC100506562, LOC100507369, LOC100507607, LOC100652777, LOC100652871, LOC100652953, LOC100996256, LOC100996259, LOC100996274, LOC100996301, LOC100996312, LOC100996318, LOC100996337, LOC100996356, LOC100996369, LOC100996394, LOC100996401, LOC100996413, LOC100996433, LOC100996451, LOC100996470, LOC100996489, LOC100996541, LOC100996547, LOC100996567, LOC100996574, LOC100996594, LOC100996610, LOC100996612, LOC100996625, LOC100996631, LOC100996643, LOC100996644, LOC100996648, LOC100996675, LOC100996689, LOC100996701, LOC100996702, LOC377711, LOC388849, LOC391322, LOC391722, LOC401052, LOC402269, LOC440243, LOC440292, LOC440563, L00554223, LOC642441, LOC642643, LOC642778, LOC642799, LOC643802, LOC644634, LOC645202, LOC645359, LOC646021, LOC646670, LOC649238, LOC728026, LOC728715, LOC728728, LOC728734, LOC728741, LOC728888, LOC729020, LOC729159, LOC729162, LOC729264, LOC729458, LOC729574, LOC729587, LOC729974, LOC730058, LOC730268, LOC731932, LOC732265, LONRF2, LPA, LPCAT3, LPGAT1, LRP5, LRP5L, LRRC16B, LRRC28, LRRC37A, LRRC37A2, LRRC37A3, LRRC37B, LRRC57, LRRC59, LRRC8B, LRRFIP1, LSM12, LSM14A, LSM2, LSM3, LSP1, LTA, LTB, LUZP6, LY6G5B, LY6G5C, LY6G6C, LY6G6D, LY6G6F, LYPLA1, LYPLA2, LYRM2, LYRM5, LYST, LYZL1, LYZL2, LYZL6, MAD1L1, MAD2L1, MAGEA10-MAGEA5, MAGEA11, MAGEA12, MAGEA2B, MAGEA4, MAGEA5, MAGEA6, MAGEA9, MAGEB2, MAGEB4, MAGEB6, MAGEC1, MAGEC3, MAGED1, MAGED2, MAGED4, MAGED4B, MAGIX, MALL, MAMDC2, MAN1A1, MAN1A2, MANBAL, MANEAL, MAP1LC3B, MAP1LC3B2, MAP2K1, MAP2K2, MAP2K4, MAP3K13, MAP7, MAPK1IP1L, MAPK6, MAPK8IP1, MAPRE1, MAPT, MARC1, MARC2, MAS1L, MASP1, MAST1, MAST2, MAST3, MAT2A, MATR3, MBD3L2, MBD3L3, MBD3L4, MBD3L5, MBLAC2, MCCD1, MCF2L2, MCFD2, MCTS1, MDC1, ME1, ME2, MEAF6, MED13, MED15, MED25, MED27, MED28, MEF2A, MEF2BNB, MEIS3, MEMO1, MEP1A, MESP1, MEST, METAP2, METTL1, METTL15, METTL21A, METTL21D, METTL2A, METTL2B, METTL5, METTL7A, METTL8, MEX3B, MEX3D, MFAP2, MFF, MFN1, MFSD2B, MGAM, MICA, MICB, MINOS1, MIPEP, MKI67, MKI67IP, MKNK1, MKRN1, MLF1IP, MLL3, MLLT10, MLLT6, MMADHC, MMP10, MMP23B, MMP3, MOB4, MOCS1, MOCS3, MOG, MORF4L1, MORF4L2, MPEG1, MPHOSPH10, MPHOSPH8, MPO, MPP7, MPPE1, MPRIP, MPV17L, MPZL1, MR1, MRC1, MRE11A, MRFAP1, MRFAP1L1, MRGPRX2, MRGPRX3, MRGPRX4, MRPL10, MRPL11, MRPL19, MRPL3, MRPL32, MRPL35, MRPL36, MRPL45, MRPL48, MRPL50, MRPL51, MRPS10, MRPS16, MRPS17, MRPS18A, MRPS18B, MRPS18C, MRPS21, MRPS24, MRPS31, MRPS33, MRPS36, MRPS5, MRRF, MRS2, MRTO4, MS4A4A, MS4A4E, MS4A6A, MS4A6E, MSANTD2, MSANTD3, MSANTD3-TMEFF1, MSH5, MSL3, MSN, MST1, MSTO1, MSX2, MT1A, MT1B, MT1E, MT1F, MT1G, MT1H, MT1M, MT1X, MT2A, MTAP, MTCH1, MTFR1, MTHFD1, MTHFD1L, MTHFD2, MTIF2, MTIF3, MTMR12, MTMR9, MTRF1L, MTRNR2L1, MTRNR2L5, MTRNR2L6, MTRNR2L8, MTX1, MUC12, MUC16, MUC19, MUC20, MUC21, MUC22, MUC5B, MUC6, MX1, MX2, MXRA5, MXRA7, MYADM, MYEOV2, MYH1, MYH11, MYH13, MYH2, MYH3, MYH4, MYH6, MYH7, MYH8, MYH9, MYL12A, MYL12B, MYL6, MYL6B, MYLK, MYO5B, MZT1, MZT2A, MZT2B, NAA40, NAALAD2, NAB1, NACA, NACA2, NACAD, NACC2, NAGK, NAIP, NAMPT, NANOG, NANOGNB, NANP, NAP1L1, NAP1L4, NAPEPLD, NAPSA, NARG2, NARS, NASP, NAT1, NAT2, NAT8, NAT8B, NBAS, NBEA, NBEAL1, NBPF1, NBPF10, NBPF11, NBPF14, NBPF15, NBPF16, NBPF4, NBPF6, NBPF7, NBPF9, NBR1, NCAPD2, NCF1, NCOA4, NCOA6, NCOR1, NCR3, NDEL1, NDST3, NDST4, NDUFA4, NDUFA5, NDUFA9, NDUFAF2, NDUFAF4, NDUFB1, NDUFB3, NDUFB4, NDUFB6, NDUFB8, NDUFB9, NDUFS5, NDUFV2, NEB, NEDD8, NEDD8-MDP1, NEFH, NEFM, NEIL2, NEK2, NETO2, NEU1, NEUROD1, NEUROD2, NF1, NFE2L3, NFIC, NFIX, NFKBIL1, NFYB, NFYC, NHLH1, NHLH2, NHP2, NHP2L1, NICN1, NIF3L1, NIP7, NIPA2, NIPAL1, NIPSNAP3A, NIPSNAP3B, NKAP, NKX1-2, NLGN4X, NLGN4Y, NLRP2, NLRP5, NLRP7, NLRP9, NMD3, NME2, NMNAT1, NOB1, NOC2L, NOL11, NOLC1, NOMO1, NOMO2, NOMO3, NONO, NOP10, NOP56, NOS2, NOTCH2, NOTCH2NL, NOTCH4, NOX4, NPAP1, NPEPPS, NPIP, NPIPL3, NPM1, NPSR1, NR2F1, NR2F2, NR3C1, NRBF2, NREP, NRM, NSA2, NSF, NSFL1C, NSMAF, NSRP1, NSUN5, NT5C3, NT5DC1, NTM, NTPCR, NUBP1, NUDC, NUDT10, NUDT11, NUDT15, NUDT16, NUDT19, NUDT4, NUDT5, NUFIP1, NUP210, NUP35, NUP50, NUS1, NUTF2, NXF2, NXF2B, NXF3, NXF5, NXPE1, NXPE2, NXT1, OAT, OBP2A, OBP2B, OBSCN, OCLN, OCM, OCM2, ODC1, OFD1, OGDH, OGDHL, OGFOD1, OGFR, OLA1, ONECUT1, ONECUT2, ONECUT3, OPCML, OPN1LW, OPN1MW, OPN1MW2, OR10A2, OR10A3, OR10A5, OR10A6, OR10C1, OR10G2, OR10G3, OR10G4, OR10G7, OR10G8, OR10G9, OR10H1, OR10H2, OR10H3, OR10H4, OR10H5, OR10J3, OR10J5, OR10K1, OR10K2, OR10Q1, OR11A1, OR11G2, OR11H1, OR11H12, OR11H2, OR12D2, OR12D3, OR13C2, OR13C4, OR13C5, OR13C9, OR13D1, OR14J1, OR1A1, OR1A2, OR1D2, OR1D5, OR1E1, OR1E2, OR1F1, OR1J1, OR1J2, OR1J4, OR1L4, OR1L6, OR1M1, OR1S1, OR1S2, OR2A1, OR2A12, OR2A14, OR2A2, OR2A25, OR2A4, OR2A42, OR2A5, OR2A7, OR2AG1, OR2AG2, OR2B2, OR2B3, OR2B6, OR2F1, OR2F2, OR2H1, OR2H2, OR2J2, OR2J3, OR2L2, OR2L3, OR2L5, OR2L8, OR2M2, OR2M5, OR2M7, OR2S2, OR2T10, OR2T2, OR2T27, OR2T29, OR2T3, OR2T33, OR2T34, OR2T35, OR2T4, OR2T5, OR2T8, OR2V1, OR2V2, OR2W1, OR3A1, OR3A2, OR3A3, OR4A15, OR4A47, OR4C12, OR4C13, OR4C46, OR4D1, OR4D10, OR4D11, OR4D2, OR4D9, OR4F16, OR4F21, OR4F29, OR4F3, OR4K15, OR4M1, OR4M2, OR4N2, OR4N4, OR4N5, OR4P4, OR4Q3, OR51A2, OR51A4, OR52E2, OR52E6, OR52E8, OR52H1, OR52I1, OR52I2, OR52J3, OR52K1, OR52K2, OR52L1, OR56A1, OR56A3, OR56A4, OR56A5, OR56B4, OR5AK2, OR5B2, OR5B3, OR5D16, OR5F1, OR5H14, OR5H2, OR5H6, OR5J2, OR5L1, OR5L2, OR5M1, OR5M10, OR5M3, OR5M8, OR5P3, OR5T1, OR5T2, OR5T3, OR5V1, OR6B2, OR6B3, OR6C6, OR7A10, OR7A5, OR7C1, OR7C2, OR7G3, OR8A1, OR8B12, OR8B2, OR8B3, OR8B8, OR8G2, OR8G5, OR8H1, OR8H2, OR8H3, OR8J1, OR8J3, OR9A2, OR9A4, OR9G1, ORC3, ORM1, ORM2, OSTC, OSTCP2, OTOA, OTOP1, OTUD4, OTUD7A, OTX2, OVOS, OXCT2, OXR1, OXT, P2RX6, P2RX7, P2RY8, PA2G4, PAAF1, PABPC1, PABPC1L2A, PABPC1L2B, PABPC3, PABPC4, PABPN1, PAEP, PAFAH1B1, PAFAH1B2, PAGE1, PAGE2, PAGE2B, PAGE5, PAICS, PAIP1, PAK2, PAM, PANK3, PARG, PARL, PARN, PARP1, PARP4, PARP8, PATL1, PBX1, PBX2, PCBD2, PCBP1, PCBP2, PCDH11X, PCDH11Y, PCDH8, PCDHA1, PCDHA11, PCDHA12, PCDHA13, PCDHA2, PCDHA3, PCDHA5, PCDHA6, PCDHA7, PCDHA8, PCDHA9, PCDHB10, PCDHB11, PCDHB12, PCDHB13, PCDHB15, PCDHB16, PCDHB4, PCDHB8, PCDHGA1, PCDHGA11, PCDHGA12, PCDHGA2, PCDHGA3, PCDHGA4, PCDHGA5, PCDHGA7, PCDHGA8, PCDHGA9, PCDHGB1, PCDHGB2, PCDHGB3, PCDHGB5, PCDHGB7, PCGF6, PCMTD1, PCNA, PCNP, PCNT, PCSK5, PCSK7, PDAP1, PDCD2, PDCD5, PDCD6, PDCD6IP, PDCL2, PDCL3, PDE4DIP, PDIA3, PDLIM1, PDPK1, PDPR, PDSS1, PDXDC1, PDZD11, PDZK1, PEBP1, PEF1, PEPD, PERP, PEX12, PEX2, PF4, PF4V1, PFDN1, PFDN4, PFDN6, PFKFB1, PFN1, PGA3, PGA4, PGA5, PGAM1, PGAM4, PGBD3, PGBD4, PGD, PGGT1B, PGK1, PGK2, PGM5, PHAX, PHB, PHC1, PHF1, PHF10, PHF2, PHF5A, PHKA1, PHLPP2, PHOSPHO1, PI3, PI4K2A, PI4KA, PIEZO2, PIGA, PIGF, PIGH, PIGN, PIGY, P1K3CA, P1K3CD, PILRA, PIN1, P1N4, PIP5K1A, PITPNB, PKD1, PKM, PKP2, PKP4, PLA2G10, PLA2G12A, PLA2G4C, PLAC8, PLAC9, PLAGL2, PLD5, PLEC, PLEKHA3, PLEKHA8, PLEKHM1, PLG, PLGLB1, PLGLB2, PL1N2, PL1N4, PLK1, PLLP, PLSCR1, PLSCR2, PLXNA1, PLXNA2, PLXNA3, PLXNA4, PM20D1, PMCH, PMM2, PMPCA, PMS2, PNKD, PNLIP, PNLIPRP2, PNMA6A, PNMA6B, PNMA6C, PNMA6D, PNO1, PNPLA4, PNPT1, POLD2, POLE3, POLH, POLR2E, POLR2J, POLR2J2, POLR2J3, POLR2M, POLR3D, POLR3G, POLR3K, POLRMT, POM121, POM121C, POMZP3, POTEA, POTEC, POTED, POTEE, POTEF, POTEH, POTEI, POTEJ, POTEM, POU3F1, POU3F2, POU3F3, POU3F4, POU4F2, POU4F3, POU5F1, PPA1, PPAT, PPBP, PPCS, PPEF2, PPFIBP1, PPIA, PPIAL4C, PPIAL4D, PPIAL4E, PPIAL4F, PPIE, PPIG, PPIL1, PP1P5K1, PPIP5K2, PPM1A, PPP1R11, PPP1R12B, PPP1R14B, PPP1R18, PPP1R2, PPP1R26, PPP1R8, PPP2CA, PPP2CB, PPP2R2D, PPP2R3B, PPP2R5C, PPP2R5E, PPP4R2, PPP5C, PPP5D1, PPP6R2, PPP6R3, PPT2, PPY, PRADC1, PRAMEF1, PRAMEF10, PRAMEF11, PRAMEF12, PRAMEF13, PRAMEF14, PRAMEF15, PRAMEF16, PRAMEF17, PRAMEF18, PRAMEF19, PRAMEF20, PRAMEF21, PRAMEF22, PRAMEF23, PRAMEF25, PRAMEF3, PRAMEF4, PRAMEF5, PRAMEF6, PRAMEF7, PRAMEF8, PRAMEF9, PRB1, PRB2, PRB3, PRB4, PRDM7, PRDM9, PRDX1, PRDX2, PRDX3, PRDX6, PRELID1, PRG4, PRH1, PRH2, PRKAR1A, PRKCI, PRKRA, PRKRIR, PRKX, PRMT1, PRMT5, PRODH, PROKR1, PROKR2, PROS1, PRPF3, PRPF38A, PRPF4B, PRPS1, PRR12, PRR13, PRR20A, PRR20B, PRR20C, PRR20D, PRR20E, PRR21, PRR23A, PRR23B, PRR23C, PRR3, PRR5-ARHGAP8, PRRC2A, PRRC2C, PRRT1, PRSS1, PRSS21, PRSS3, PRSS41, PRSS42, PRSS48, PRUNE, PRY, PRY2, PSAT1, PSG1, PSG11, PSG2, PSG3, PSG4, PSG5, PSG6, PSG8, PSG9, PSIP1, PSMA6, PSMB3, PSMB5, PSMB8, PSMB9, PSMC1, PSMC2, PSMC3, PSMC5, PSMC6, PSMD10, PSMD12, PSMD2, PSMD4, PSMD7, PSMD8, PSME2, PSORS1C1, PSORS1C2, PSPH, PTBP1, PTCD2, PTCH1, PTCHD3, PTCHD4, PTEN, PTGES3, PTGES3L-AARSD1, PTGR1, PTMA, PTMS, PTOV1, PTP4A1, PTP4A2, PTPN11, PTPN2, PTPN20A, PTPN20B, PTPRD, PTPRH, PTPRM, PTPRN2, PTPRU, PTTG1, PTTG2, PVRIG, PVRL2, PWWP2A, PYGB, PYGL, PYHIN1, PYROXD1, PYURF, PYY, PZP, QRSL1, R3HDM2, RAB11A, RAB11FIP1, RAB13, RAB18, RAB1A, RAB1B, RAB28, RAB31, RAB40AL, RAB40B, RAB42, RAB43, RAB5A, RAB5C, RAB6A, RAB6C, RAB9A, RABGEF1, RABGGTB, RABL2A, RABL2B, RABL6, RAC1, RACGAP1, RAD1, RAD17, RAD21, RAD23B, RAD51AP1, RAD54L2, RAET1G, RAET1L, RALA, RALBP1, RALGAPA1, RAN, RANBP1, RANBP17, RANBP2, RANBP6, RAP1A, RAP1B, RAP1GDS1, RAP2A, RAP2B, RARS, RASA4, RASA4B, RASGRP2, RBAK, RBAK-LOC389458, RBBP4, RBBP6, RBM14-RBM4, RBM15, RBM17, RBM39, RBM4, RBM43, RBM48, RBM4B, RBM7, RBM8A, RBMS1, RBMS2, RBMX, RBMX2, RBMXL1, RBMXL2, RBMY1A1, RBMY1B, RBMY1D, RBMY1E, RBMY1F, RBMY1J, RBPJ, RCBTB1, RCBTB2, RCC2, RCN1, RCOR2, RDBP, RDH16, RDM1, RDX, RECQL, REG1A, REG1B, REG3A, REG3G, RELA, RERE, RETSAT, REV1, REXO4, RFC3, RFESD, RFK, RFPL1, RFPL2, RFPL3, RFPL4A, RFTN1, RFWD2, RGL2, RGPD1, RGPD2, RGPD3, RGPD4, RGPD5, RGPD6, RGPD8, RGS17, RGS19, RGS9, RHBDF1, RHCE, RHD, RHEB, RHOQ RHOT1, RHOXF2, RHOXF2B, RHPN2, RIMBP3, RIMBP3B, RIMBP3C, RIMKLB, RING1, RLIM, RLN1, RLN2, RLTPR, RMND1, RMND5A, RNASE2, RNASE3, RNASE7, RNASE8, RNASEH1, RNASET2, RNF11, RNF123, RNF126, RNF13, RNF138, RNF14, RNF141, RNF145, RNF152, RNF181, RNF2, RNF216, RNF39, RNF4, RNF5, RNF6, RNFT1, RNMTL1, RNPC3, RNPS1, ROBO2, ROCK1, ROCK2, ROPN1, ROPN1B, RORA, RP9, RPA2, RPA3, RPAP2, RPE, RPF2, RPGR, RPL10, RPL10A, RPL10L, RPL12, RPL13, RPL14, RPL15, RPL17, RPL17-C18ORF32, RPL18A, RPL19, RPL21, RPL22, RPL23, RPL23A, RPL24, RPL26, RPL26L1, RPL27, RPL27A, RPL29, RPL3, RPL30, RPL31, RPL32, RPL35, RPL35A, RPL36, RPL36A, RPL36A-HNRNPH2, RPL36AL, RPL37, RPL37A, RPL39, RPL4, RPL41, RPL5, RPL6, RPL7, RPL7A, RPL7L1, RPL8, RPL9, RPLP0, RPLP1, RPP21, RPS10, RPS10-NUDT3, RPS11, RPS13, RPS14, RPS15, RPS15A, RPS16, RPS17, RPS17L, RPS18, RPS19, RPS2, RPS20, RPS23, RPS24, RPS25, RPS26, RPS27, RPS27A, RPS28, RPS3, RPS3A, RPS4X, RPS4Y1, RPS4Y2, RPS5, RPS6, RPS6KB1, RPS7, RPS8, RPS9, RPSA, RPTN, RRAGA, RRAGB, RRAS2, RRM2, RRN3, RRP7A, RSL24D1, RSPH10B, RSPH10B2, RSPO2, RSRC1, RSU1, RTEL1, RTN3, RTN4IP1, RTN4R, RTP1, RTP2, RUFY3, RUNDC1, RUVBL2, RWDD1, RWDD4, RXRB, RYK, S100A11, S100A7L2, SAA1, SAA2, SAA2-SAA4, SAE1, SAFB, SAFB2, SAGE1, SALL1, SALL4, SAMD12, SAMD9, SAMD9L, SAP18, SAP25, SAP30, SAPCD1, SAPCD2, SAR1A, SATL1, SAV1, SAYSD1, SBDS, SBF1, SCAMP1, SCAND3, SCD, SCGB1D1, SCGB1D2, SCGB1D4, SCGB2A1, SCGB2A2, SCGB2B2, SCN10A, SCN1A, SCN2A, SCN3A, SCN4A, SCN5A, SCN9A, SCOC, SCXA, SCXB, SCYL2, SDAD1, SDCBP, SDCCAG3, SDHA, SDHB, SDHC, SDHD, SDR42E1, SEC11A, SEC14L1, SEC14L4, SEC14L6, SEC61B, SEC63, SELT, SEMA3E, SEMG1, SEMG2, SEPHS1, SEPHS2, SEPT14, SEPT7, SERBP1, SERF1A, SERF1B, SERF2, SERHL2, SERPINB3, SERPINB4, SERPINH1, SET, SETD8, SF3A2, SF3A3, SF3B14, SF3B4, SFR1, SFRP4, SFTA2, SFTPA1, SFTPA2, SH2D1B, SH3BGRL3, SH3GL1, SHANK2, SHC1, SHCBP1, SHFM1, SHH, SHISA5, SHMT1, SHOX, SHQ1, SHROOM2, SIGLEC10, SIGLEC11, SIGLEC12, SIGLEC14, SIGLECS, SIGLEC6, SIGLEC7, SIGLEC8, SIGLEC9, SIMC1, SIN3A, SIRPA, SIRPB1, SIRPG, SIX1, SIX2, SKA2, SKIV2L, SKOR2, SKP1, SKP2, SLAIN2, SLAMF6, SLC10A5, SLC16A14, SLC16A6, SLC19A3, SLC22A10, SLC22A11, SLC22A12, SLC22A24, SLC22A25, SLC22A3, SLC22A4, SLC22A5, SLC22A9, SLC25A13, SLC25A14, SLC25A15, SLC25A20, SLC25A29, SLC25A3, SLC25A33, SLC25A38, SLC25A47, SLC25A5, SLC25A52, SLC25A53, SLC25A6, SLC29A4, SLC2A13, SLC2A14, SLC2A3, SLC31A1, SLC33A1, SLC35A4, SLC35E1, SLC35E2, SLC35E2B, SLC35G3, SLC35G4, SLC35G5, SLC35G6, SLC36A1, SLC36A2, SLC39A1, SLC39A7, SLC44A4, SLC4A1AP, SLC52A1, SLC52A2, SLC5A6, SLC5A8, SLC6A14, SLC6A6, SLC6A8, SLC7A5, SLC8A2, SLC8A3, SLC9A2, SLC9A4, SLC9A7, SLCO1B1, SLCO1B3, SLCO1B7, SLFN11, SLFN12, SLFN12L, SLFN13, SLFN5, SLIRP, SLMO2, SLX1A, SLX1B, SMARCE1, SMC3, SMC5, SMEK2, SMG1, SMN1, SMN2, SMR3A, SMR38, SMS, SMU1, SMURF2, SNAI1, SNAPC4, SNAPC5, SNF8, SNRNP200, SNRPA1, SNRPB2, SNRPC, SNRPD1, SNRPD2, SNRPE, SNRPG, SNRPN, SNW1, SNX19, SNX25, SNX29, SNX5, SNX6, SOCS5, SOCS6, SOGA1, SOGA2, SON, SOX1, SOX10, 50X14, SOX2, SOX30, SOX5, SOX9, SP100, SP140, SP140L, SP3, SP5, SP8, SP9, SPACA5, SPACA5B, SPACA7, SPAG11A, SPAG11B, SPANXA1, SPANXB1, SPANXD, SPANXN2, SPANXN5, SPATA16, SPATA20, SPATA31A1, SPATA31A2, SPATA31A3, SPATA31A4, SPATA31A5, SPATA31A6, SPATA31A7, SPATA31C1, SPATA31C2, SPATA31D1, SPATA31D3, SPATA31D4, SPATA31E1, SPCS2, SPDYE1, SPDYE2, SPDYE2L, SPDYE3, SPDYE4, SPDYE5, SPDYE6, SPECC1, SPECC1L, SPHAR, SPIC, SPIN1, SPIN2A, SPIN2B, SPOPL, SPPL2A, SPPL2C, SPR, SPRR1A, SPRR1B, SPRR2A, SPRR2B, SPRR2D, SPRR2E, SPRR2F, SPRY3, SPRYD4, SPTLC1, SRD5A1, SRD5A3, SREK1IP1, SRGAP2, SRP14, SRP19, SRP68, SRP72, SRP9, SRPK1, SRPK2, SRRM1, SRSF1, SRSF10, SRSF11, SRSF3, SRSF6, SRSF9, SRXN1, SS18L2, SSB, SSBP2, SSBP3, SSBP4, SSNA1, SSR3, SSX1, SSX2, SSX2B, SSX3, SSX4, SSX4B, SSX5, SSX7, ST13, ST3GAL1, STAG3, STAR, STAT5A, STAT5B, STAU1, STAU2, STBD1, STEAP1, STEAP1B, STH, STIP1, STK19, STK24, STK32A, STMN1, STMN2, STMN3, STRADB, STRAP, STRC, STRN, STS, STUB1, STX18, SUB1, SUCLA2, SUCLG2, SUDS3, SUGP1, SUGT1, SULT1A1, SULT1A2, SULT1A3, SULT1A4, SUMF2, SUMO1, SUMO2, SUPT16H, SUPT4H1, SUSD2, SUZ12, SVIL, SWI5, SYCE2, SYNCRIP, SYNGAP1, SYNGR2, SYT14, SYT15, SYT2, SYT3, SZRD1, TAAR6, TAAR8, TACC1, TADA1, TAF1, TAF15, TAF1L, TAF4B, TAF5L, TAF9, TAF9B, TAGLN2, TALDO1, TANC2, TAP1, TAP2, TAPBP, TARBP2, TARDBP, TARP, TAS2R19, TAS2R20, TAS2R30, TAS2R39, TAS2R40, TAS2R43, TAS2R46, TAS2R50, TASP1, TATDN1, TATDN2, TBC1D26, TBC1D27, TBC1D28, TBC1D29, TBC1D2B, TBC1D3, TBC1D3B, TBC1D3C, TBC1D3F, TBC1D3G, TBC1D3H, TBCA, TBCCD1, TBL1X, TBL1XR1, TBL1Y, TBPL1, TBX20, TC2N, TCEA1, TCEAL2, TCEAL3, TCEAL5, TCEB1, TCEB2, TCEB3B, TCEB3C, TCEB3CL, TCEB3CL2, TCERG1L, TCF19, TCF3, TCHH, TCL1B, TCOF1, TCP1, TCP10, TCP10L, TCP10L2, TDG, TDGF1, TDRD1, TEAD1, TEC, TECR, TEKT4, TERF1, TERF2IP, TET1, TEX13A, TEX13B, TEX28, TF, TFB2M, TFDP3, TFG, TGIF1, TGIF2, TGIF2LX, TGIF2LY, THAP3, THAP5, THEM4, THOC3, THRAP3, THSD1, THUMPD1, TIMM17B, TIMM23B, TIMM8A, TIMM8B, TIMP4, TIPIN, TJAP1, TJP3, TLE1, TLE4, TLK1, TLK2, TLL1, TLR1, TLR6, TMA16, TMA7, TMC6, TMCC1, TMED10, TMED2, TMEM126A, TMEM128, TMEM132B, TMEM132C, TMEM14B, TMEM14C, TMEM161B, TMEM167A, TMEM183A, TMEM183B, TMEM185A, TMEM185B, TMEM189-UBE2V1, TMEM191B, TMEM191C, TMEM230, TMEM231, TMEM236, TMEM242, TMEM251, TMEM254, TMEM30B, TMEM47, TMEM69, TMEM80, TMEM92, TMEM97, TMEM98, TMLHE, TMPRSS11E, TMSB10, TMSB15A, TMSB15B, TMSB4X, TMSB4Y, TMTC1, TMTC4, TMX1, TMX2, TNC, TNF, TNFRSF10A, TNFRSF10B, TNFRSF10C, TNFRSF10D, TNFRSF13B, TNFRSF14, TNIP2, TNN, TNPO1, TNRC18, TNXB, TOB2, TOE1, TOMM20, TOMM40, TOMM6, TOMM7, TOP1, TOP3B, TOR1B, TOR3A, TOX4, TP53TG3, TP53TG3B, TP53TG3C, TPD52L2, TPI1, TPM3, TPM4, TPMT, TPRKB, TPRX1, TPSAB1, TPSB2, TPSD1, TPT1, TPTE, TPTE2, TRA2A, TRAF6, TRAPPC2, TRAPPC2L, TREH, TREML2, TREML4, TRIM10, TRIM15, TRIM16, TRIM16L, TRIM26, TRIM27, TRIM31, TRIM38, TRIM39, TRIM39-RPP21, TRIM40, TRIM43, TRIM43B, TRIM48, TRIM49, TRIM49B, TRIM49C, TRIM49DP, TRIM49L1, TRIM50, TRIM51, TRIM51GP, TRIM60, TRIM61, TRIM64, TRIM64B, TRIM64C, TRIM73, TRIM74, TRIM77P, TRIP11, TRMT1, TRMT11, TRMT112, TRMT2B, TRNT1, TRO, TRPA1, TRPC6, TRPV5, TRPV6, TSC22D3, TSEN15, TSEN2, TSPAN11, TSPY1, TSPY10, TSPY2, TSPY3, TSPY4, TSPY8, TSPYL1, TSPYL6, TSR1, TSSK1B, TSSK2, TTC28, TTC3, TTC30A, TTC30B, TTC4, TTL, TTLL12, TTLL2, TTN, TUBA1A, TUBA1B, TUBA1C, TUBA3C, TUBA3D, TUBA3E, TUBA4A, TUBA8, TUBB, TUBB2A, TUBB2B, TUBB3, TUBB4A, TUBB4B, TUBB6, TUBB8, TUBE1, TUBG1, TUBG2, TUBGCP3, TUBGCP6, TUFM, TWF1, TWIST2, TXLNG, TXN2, TXNDC2, TXNDC9, TYR, TYRO3, TYW1, TYW1B, U2AF1, UAP1, UBA2, UBA5, UBD, UBE2C, UBE2D2, UBE2D3, UBE2D4, UBE2E3, UBE2F, UBE2H, UBE2L3, UBE2M, UBE2N, UBE2Q2, UBE2S, UBE2V1, UBE2V2, UBE2W, UBE3A, UBFD1, UBQLN1, UBQLN4, UBTFL1, UBXN2B, UFD1L, UFM1, UGT1A10, UGT1A3, UGT1A4, UGT1A5, UGT1A7, UGT1A8, UGT1A9, UGT2A1, UGT2A2, UGT2A3, UGT2B10, UGT2B11, UGT2B15, UGT2B17, UGT2B28, UGT2B4, UGT2B7, UGT3A2, UHRF1, UHRF2, ULBP1, ULBP2, ULBP3, ULK4, UNC93A, UNC93B1, UPF3A, UPK3B, UPK3BL, UQCR10, UQCRB, UQCRFS1, UQCRH, UQCRQ USP10, USP12, USP13, USP17L10, USP17L11, USP17L12, USP17L13, USP17L15, USP17L17, USP17L18, USP17L19, USP17L1P, USP17L2, USP17L20, USP17L21, USP17L22, USP17L24, USP17L25, USP17L26, USP17L27, USP17L28, USP17L29, USP17L3, USP17L30, USP17L4, USP17L5, USP17L7, USP17L8, USP18, USP22, USP32, USP34, USP6, USP8, USP9X, USP9Y, UTP14A, UTP14C, UTP18, UTP6, VAMPS, VAMP7, VAPA, VARS, VARS2, VCX, VCX2, VCX3A, VCX3B, VCY, VCY1B, VDAC1, VDAC2, VDAC3, VENTX, VEZF1, VKORC1, VKORC1L1, VMA21, VN1R4, VNN1, VOPP1, VPS26A, VPS35, VPS37A, VPS51, VPS52, VSIG10, VTCN1, VTI1B, VWA5B2, VWA7, VWA8, VWF, WARS, WASF2, WASF3, WASH1, WBP1, WBP11, WBP1L, WBSCR16, WDR12, WDR45, WDR45L, WDR46, WDR49, WDR59, WDR70, WDR82, WDR89, WFDC10A, WFDC10B, WHAMM, WHSC1L1, WIPI2, WIZ, WNT3, WNT3A, WNTSA, WNT5B, WNT9B, WRN, WTAP, WWC2, WWC3, WWP1, XAGE1A, XAGE1B, XAGE1C, XAGE1D, XAGE1E, XAGE2, XAGE3, XAGE5, XBP1, XCL1, XCL2, XG, XIAP, XKR3, XKR8, XKRY, XKRY2, XPO6, XPOT, XRCC6, YAP1, YBX1, YBX2, YES1, YME1L1, YPEL5, YTHDC1, YTHDF1, YTHDF2, YWHAB, YWHAE, YWHAQ YWHAZ, YY1, YY1AP1, ZAN, ZBED1, ZBTB10, ZBTB12, ZBTB22, ZBTB44, ZBTB45, ZBTB8OS, ZBTB9, ZC3H11A, ZC3H12A, ZCCHC10, ZCCHC12, ZCCHC17, ZCCHC18, ZCCHC2, ZCCHC7, ZCCHC9, ZCRB1, ZDHHC11, ZDHHC20, ZDHHC3, ZDHHC8, ZEB2, ZFAND5, ZFAND6, ZFP106, ZFP112, ZFP14, ZFP57, ZFP64, ZFP82, ZFR, ZFX, ZFY, ZFYVE1, ZFYVE9, ZIC1, ZIC2, ZIC3, ZIC4, ZIK1, ZKSCAN3, ZKSCAN4, ZMIZ1, ZMIZ2, ZMYM2, ZMYM5, ZNF100, ZNF101, ZNF107, ZNF114, ZNF117, ZNF12, ZNF124, ZNF131, ZNF135, ZNF14, ZNF140, ZNF141, ZNF146, ZNF155, ZNF160, ZNF167, ZNF17, ZNF181, ZNF185, ZNF20, ZNF207, ZNF208, ZNF212, ZNF221, ZNF222, ZNF223, ZNF224, ZNF225, ZNF226, ZNF229, ZNF230, ZNF233, ZNF234, ZNF235, ZNF248, ZNF253, ZNF254, ZNF257, ZNF259, ZNF26, ZNF264, ZNF266, ZNF267, ZNF280A, ZNF280B, ZNF282, ZNF283, ZNF284, ZNF285, ZNF286A, ZNF286B, ZNF300, ZNF302, ZNF311, ZNF317, ZNF320, ZNF322, ZNF323, ZNF324, ZNF324B, ZNF33A, ZNF33B, ZNF341, ZNF347, ZNF35, ZNF350, ZNF354A, ZNF354B, ZNF354C, ZNF366, ZNF37A, ZNF383, ZNF396, ZNF41, ZNF415, ZNF416, ZNF417, ZNF418, ZNF419, ZNF426, ZNF429, ZNF43, ZNF430, ZNF431, ZNF433, ZNF439, ZNF44, ZNF440, ZNF441, ZNF442, ZNF443, ZNF444, ZNF451, ZNF460, ZNF468, ZNF470, ZNF479, ZNF480, ZNF484, ZNF486, ZNF491, ZNF492, ZNF506, ZNF528, ZNF532, ZNF534, ZNF543, ZNF546, ZNF547, ZNF548, ZNF552, ZNF555, ZNF557, ZNF558, ZNF561, ZNF562, ZNF563, ZNF564, ZNF57, ZNF570, ZNF578, ZNF583, ZNF585A, ZNF585B, ZNF586, ZNF587, ZNF587B, ZNF589, ZNF592, ZNF594, ZNF595, ZNF598, ZNF605, ZNF607, ZNF610, ZNF613, ZNF614, ZNF615, ZNF616, ZNF620, ZNF621, ZNF622, ZNF625, ZNF626, ZNF627, ZNF628, ZNF646, ZNF649, ZNF652, ZNF655, ZNF658, ZNF665, ZNF673, ZNF674, ZNF675, ZNF676, ZNF678, ZNF679, ZNF680, ZNF681, ZNF682, ZNF69, ZNF700, ZNF701, ZNF705A, ZNF705B, ZNF705D, ZNF705E, ZNF705G, ZNF706, ZNF708, ZNF709, ZNF710, ZNF714, ZNF716, ZNF717, ZNF718, ZNF720, ZNF721, ZNF726, ZNF727, ZNF728, ZNF729, ZNF732, ZNF735, ZNF736, ZNF737, ZNF746, ZNF747, ZNF749, ZNF75A, ZNF75D, ZNF761, ZNF763, ZNF764, ZNF765, ZNF766, ZNF770, ZNF773, ZNF775, ZNF776, ZNF777, ZNF780A, ZNF780B, ZNF782, ZNF783, ZNF791, ZNF792, ZNF799, ZNF805, ZNF806, ZNF808, ZNF812, ZNF813, ZNF814, ZNF816, ZNF816-ZNF321P, ZNF823, ZNF829, ZNF83, ZNF836, ZNF84, ZNF841, ZNF844, ZNF845, ZNF850, ZNF852, ZNF878, ZNF879, ZNF880, ZNF90, ZNF91, ZNF92, ZNF93, ZNF98, ZNF99, ZNRD1, ZNRF2, ZP3, ZRSR2, ZSCAN5A, ZSCAN5B, ZSCAN5D, ZSWIM5, ZXDA, ZXDB, ZXDC, portions thereof, modified forms thereof or combinations thereof. In certain embodiments, a desired nucleic acid or gene is selected from one or more of ABL1, ANGPTL4, APOB, APOC3, ASGR1, BRCA1, BRCA2, BRAF, CD19, CD36, CFTR, DMD, FMR1, HTT, TCF4, CEP290, G6PC, PCSK9, EYA4, GJB2, SLC26A4, ABCA4, CNGA3, CNGB3, MERTK, MYO7A, REP1, RHO, RPE65, RS1, USH2A, PD1, PD-L1 (or CD274), EGFR, RAF, RAS, portions thereof, and modified forms thereof.

In some embodiments, a synthetic nucleic acid comprises a first nucleic acid sequence comprising about 1000 or more contiguous nucleotides, wherein the first nucleic acid sequence encodes a codon-Adapted Nuclear Argonaute protein (ANAGO) that is a polypeptide, capable of editing a target nucleic acid sequence within a human cell in the presence of a donor nucleic acid without a guide nucleic acid, wherein the ANAGO is species-specific to the human, wherein the ANAGO is attached to a coding sequence of a nuclear localization signal (NLS) peptide; wherein the first nucleic acid sequence is produced by modifying a second nucleic acid sequence of a microbial species, and wherein the second nucleic acid sequence comprises a coding region that is capable of encoding a microbial Argonaute protein that has endonuclease activities in a microorganism; wherein the modifying comprises replacing microbial preferred codons of the second nucleic acid sequence with codons that have preferential usage in the human cell, and wherein the first nucleic acid sequence comprises at least 85% identity to the nucleic acid sequence of SEQ ID NO:1; SEQ ID NO:2; SEQ ID NO:3, or SEQ ID NO:4. In some embodiments, the human cell is a human stem cell.

In certain embodiments a method of editing a genome of an organism or a cell comprises introducing one or more ANAGO, or a composition thereof described herein, into one or more cells. In certain embodiments a method of editing a genome of an organism or cell is a method of modifying a target sequence in a genome of a cell, organism or subject. In certain embodiments a method of editing a genome of an organism or cell comprises introducing one or more ANAGO described herein into one or more cells. One or more nucleic acid can be introduced into one or more cells by any suitable method.

In some embodiments, a method described herein comprises introducing into a eukaryotic cell, (i) an ANAGO in a form of protein or in vitro transcribed messenger RNA; and (ii) one or more nucleic acid donors (one or more donor sequences). In some embodiments, a donor nucleic acid comprises a desired nucleic acid flanked by a 5′-flanking sequence and a 3′ flanking sequence. In some embodiments, a method described herein comprises introducing into a human cell, (i) an ANAGO having a sequence encoded by e.g., a nucleic acid of SEQ ID NO:1, nucleic acid of SEQ ID NO:2, nucleic acid of SEQ ID NO:3, or nucleic acid of SEQ ID NO:4, and (ii) a donor nucleic acid described herein. In certain embodiments the donor nucleic acid sequence comprises a desired nucleic acid. In some embodiments, the method induces, results in, or provides a modification of a target sequence. A modification of a target sequence may comprise an insertion, deletion, or replacement of one or more nucleotides of the target sequence. In some embodiments, a modification of a target sequence comprises an insertion, deletion or replacement of a single nucleotide of the target sequence. In certain embodiments, the method results in integration or insertion of a desired nucleic acid into the genome of the cell. In certain embodiments, the method results in replacement of a dysfunctional or mutated endogenous gene, or portion thereof, in the genome of a cell, with a wild-type, modified and/or a more functional gene. In certain embodiments, the method results in targeted disruption of an endogenous or wild type gene in the genome of the cell.

In some embodiments, a method of editing a genome of a human cell comprises introducing into the human cell (i) a species-specific ANAGO encoded by the first nucleic acid sequence described herein, or an in vitro messenger RNA transcribed by the first nucleic acid sequence described herein; and (ii) a donor nucleic acid comprising: a desired nucleic acid sequence to be introduced into a target sequence, wherein introducing the desired nucleic acid sequence is induced by an ANAGO, a 5′-flanking sequence, and a 3′-flanking sequence, wherein the 5′-flanking sequence and the 3′-flanking sequence are located on opposite sides of the desired nucleic acid sequence and independently comprise at least 10 consecutive nucleotides that are at least 90% identical to the target sequence located in the genome of the human cell; and wherein the first synthetic nucleic acid sequence comprises at least 85% identity to the nucleic acid sequence of SEQ ID NO:1, SEQ ID NO:2, SEQ ID NO:3, or SEQ ID NO:4. In some embodiments, the human cell is a human stem cell.

A cell may be contacted with ANAGO, and a donor sequence at the same time, or at different times. For example, a cell may be contacted with an ANAGO, followed by contacting the cell with a donor sequence within a time range of a week, 0 to 72 hours, 0 to 24 hours, 0 to 12 hours, 0 to 6 hours or 0 to 4 hours. A cell may be contacted with the nucleic acids described herein in any order.

### Pharmaceutical Compositions

In some embodiments a pharmaceutical composition comprises one or more nucleic acids described herein.

In some embodiments, a pharmaceutical composition comprises one or more species-specific ANAGO described herein.

In some embodiments, a composition comprises one or more species-specific ANAGO, one or more nucleic acid donors, one or more NLS, one or more short peptide tag sequence (TAG) described herein. In some embodiments, a composition comprises one or more human ANAGO, one or more nucleic acid donors, one or more NLS, one or more TAG described herein. In some embodiments, a composition comprises one or more human ANAGO encoded with nucleic acid sequence of SEQ ID NO:1, SEQ ID NO:2, SEQ ID NO:3, or SEQ ID NO:4, one or more nucleic acid donors, one or more NLS, one or more TAG described herein. In some embodiments, a composition comprises one or more human ANAGO encoded with nucleic acid sequence of SEQ ID NO:1, one or more nucleic acid donors, one or more NLS, one or more TAG described herein. In some embodiments, a composition comprises one or more human ANAGO encoded with nucleic acid sequence of SEQ ID NO:2, one or more nucleic acid donors, one or more NLS, one or more TAG described herein. In some embodiments, a composition comprises one or more human ANAGO encoded with nucleic acid sequence of SEQ ID NO:3, one or more nucleic acid donors, one or more NLS, one or more TAG described herein. In some embodiments, a composition comprises one or more human ANAGO encoded with nucleic acid sequence of SEQ ID NO:4, one or more nucleic acid donors, one or more NLS, one or more TAG described herein.

In some embodiments, a composition comprises a synthetic nucleic acid described herein and a donor nucleic acid comprising: (i) a desired nucleic acid sequence to be introduced into a target sequence, wherein introducing the desired nucleic acid sequence is induced by an ANAGO; (ii) a 5′-flanking sequence; and (iii) a 3′-flanking sequence, wherein the 5′-flanking sequence and the 3′-flanking sequence independently comprise at least 10 consecutive nucleotides that are at least 90% identical to the target sequence located in the genome of a human cell. In some embodiments, the human cell is a human stem cell.

An ANAGO, including the protein or the nucleic acid encoding the protein, may be in a pharmaceutical composition. The exact formulation and route of administration can be chosen by the individual physician in view of the patient's condition. See, e.g., Fingl et al. 1975, in “The Pharmacological Basis of Therapeutics,” Ch. 1, p. 1; which is incorporated herein by reference in its entirety. The pharmaceutical composition or formulation may be administered by any suitable route of delivery including, but not limited to, topical or local (e.g., eyedrop, transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranially, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), subretinal injection, the like or combinations thereof.

Appropriate excipients for use in a pharmaceutical composition comprising an ANAGO, including the protein or the nucleic acid encoding the protein, may include, for example, one or more carriers, binders, fillers, vehicles, tonicity agents, buffers, disintegrates, surfactants, dispersion or suspension aids, thickening or emulsifying agents, preservatives, lubricants and the like or combinations thereof, as suited to a particular dosage from desired. Remington's Pharmaceutical Sciences, 18th Ed., A. R. Gennaro, ed., Mack Publishing Company (1995) discloses various carriers used in formulating pharmaceutically acceptable compositions and known techniques for the preparation thereof. This document is incorporated herein by reference in its entirety.

In addition to water or another solvent, a liquid dosage form for IV, injection, topical, or oral administration to a mammal, including a human being, may contain excipients such as bulking agents (such as mannitol, lactose, sucrose, trehalose, sorbitol, glucose, raffinose, glycine, histidine, polyvinylpyrrolidone, etc.), tonicity agents (e.g. dextrose, glycerin, mannitol, sodium chloride, etc.), buffers (e.g. acetate, e.g. sodium acetate, acetic acid, ammonium acetate, ammonium sulfate, ammonium hydroxide, citrate, tartrate, phosphate, triethanolamine, arginine, aspartate, benzenesulfonic acid, benzoate, bicarbonate, borate, carbonate, succinate, sulfate, tartrate, tromethamine, diethanolamine etc.), preservatives (e.g. phenol, m-cresol, a paraben, such as methylparaben, propylparaben, butylparaben, myristyl gamma-picolinium chloride, benzalkonium chloride, benzethonium chloride, benzyl alcohol, 2-penoxyethanol, chlorobutanol, thimerosal, phenymercuric salts, etc.), surfactants (e.g. polyoxyethylene sorbitan monooleate or Tween 80, sorbitan monooleate polyoxyethylene sorbitan monolaurate or Tween 20, lecithin, a polyoxyethylene-polyoxypropylen copolymer, etc.), additional solvents (e.g. propylene glycol, glycerin, ethanol, polyethylene glycol, sorbitol, dimethylacetamide, Cremophor EL, benzyl benzoate, castor oil, cottonseed oil, N-methyl-2-pyrrolidone, PEG, PEG 300, PEG 400, PEG 600, PEG 600, PEG 3350, PEG 400, poppyseed oil, propylene glycol, safflower oil, vegetable oil, etc.) chelating agents (such as calcium disodium EDTA, disodium EDTA, sodium EDTA, calcium versetamide Na, calteridol, DTPA), or other excipients.

In certain embodiments, the amount of a nucleic acid described herein can be any sufficient amount to prevent, treat, reduce the severity of, delay the onset of, or alleviate a symptom of a disease as contemplated herein or a specific indication as described herein.

Certain embodiments provide pharmaceutical compositions suitable for use in the technology, which include compositions where the active ingredients are contained in an amount effective to achieve its intended purpose. A “therapeutically effective amount” means an amount sufficient to prevent, treat, reduce the severity of, delay the onset of, or inhibit a symptom of a disease. The symptom can be a symptom already occurring or expected to occur. Determination of a therapeutically effective amount is well within the capability of those skilled in the art, especially in light of the detailed disclosure provided herein.

The term “an amount sufficient” as used herein refers to the amount or quantity of an active agent (e.g., a nucleic acid described herein, an ANAGO described herein, an anti-bacterial medication, and/or a combination of these active agents) presents in a pharmaceutical composition that is determined to be high enough to prevent, treat, reduce the severity of, delay the onset of, or inhibit a symptom of a disease and low enough to minimize unwanted adverse reactions.

The ANAGO, in a form of protein or nucleic acid, and compositions comprising ANAGO as described herein can be administered at a suitable dose, e.g., at a suitable volume and concentration depending on the route of administration. Within certain embodiments, dosages of administered ANAGO can be from 0.01-500 mg/kg (e.g., per kg body weight of a subject), such as 0.01-0.02 mg/kg, 0.02-0.03 mg/kg, 0.03-0.04 mg/kg, 0.04-0.05 mg/kg, 0.05-0.06 mg/kg, 0.06-0.07 mg/kg, 0.07-0.08 mg/kg, 0.08-0.09 mg/kg, 0.09-0.1 mg/kg, 0.1-0.2 mg/kg, 0.2-0.3 mg/kg, 0.3-0.4 mg/kg, 0.4-0.5 mg/kg, 0.5-0.6 mg/kg, 0.6-0.7 mg/kg, 0.7-0.8 mg/kg, 0.8-0.9 mg/kg, 0.9-1 mg/kg, 1-2 mg/kg, 2-3 mg/kg, 3-4 mg/kg, 4-5 mg/kg, 5-6 mg/kg, 6-7 mg/kg, 7-8 mg/kg, 8-9 mg/kg, 9-10 mg/kg, 10-20 mg/kg, 20-30 mg/kg, 30-40 mg/kg, 40-50 mg/kg, 50-60 mg/kg, 60-70 mg/kg, 70-80 mg/kg, 80-90 mg/kg, 90-100 mg/kg, 100-200 mg/kg, 200-300 mg/kg, 300-400 mg/kg, 400-500 mg/kg, 0.01-0.1 mg/kg, 0.1-1 mg/kg, 1-10 mg/kg, 10-100 mg/kg, or 100-500 mg/kg. In some embodiments a nucleic acid described herein comprises one or more distinguishable identifiers. Any suitable distinguishable identifier and/or detectable identifier can be used for a composition or method described herein. In certain embodiments a distinguishable identifier can be directly or indirectly associated with (e.g., bound to) a nucleic acid described herein. For example, a distinguishable identifier can be covalently or non-covalently bound to a nucleic acid described herein. In some embodiments a distinguishable identifier is bound to or associated with a nucleic acid described herein and/or a member of binding pair that is covalently or non-covalently bound to a nucleic acid described herein. In some embodiments a distinguishable identifier is reversibly associated with a nucleic acid described herein. In certain embodiments a distinguishable identifier that is reversibly associated with a nucleic acid described herein can be removed from a nucleic acid described herein using a suitable method (e.g., by increasing salt concentration, denaturing, washing, adding a suitable solvent and/or salt, adding a suitable competitor, and/or by heating).

In some embodiments a distinguishable identifier is a label. In some embodiments a nucleic acid described herein comprises a detectable label, non-limiting examples of which include a radiolabel (e.g., an isotope), a metallic label, a fluorescent label, a chromophore, a chemiluminescent label, an electrochemiluminescent label (e.g., Origen™), a phosphorescent label, a quencher (e.g., a fluorophore quencher), a fluorescence resonance energy transfer (FRET) pair (e.g., donor and acceptor), a dye, a protein (e.g., an enzyme (e.g., alkaline phosphatase and horseradish peroxidase), an antibody, an antigen or part thereof, a linker, a member of a binding pair), an enzyme substrate, a small molecule (e.g., biotin, avidin), a mass tag, quantum dots, nanoparticles, the like or combinations thereof. Any suitable fluorophore or light emitting material can be used as a label. A light emitting label can be detected and/or quantitated by a variety of suitable techniques such as, for example, flow cytometry, gel electrophoresis, protein-chip analysis (e.g., any chip methodology), microarray, mass spectrometry, cytofluorimetric analysis, fluorescence microscopy, confocal laser scanning microscopy, laser scanning cytometry, the like and combinations thereof.

### Binding Pairs

In some embodiments a nucleic acid, or composition described herein comprises one or more binding pairs. In some embodiments a binding pair comprises at least two members (e.g., molecules) that bind non-covalently to (e.g., associate with) each other. Members of a binding pair often bind specifically to each other. Members of a binding pair often bind reversibly to each other, for example where the association of two members of a binding pair can be dissociated by a suitable method. Any suitable binding pair, or members thereof, can be utilized for a composition or method described herein. Non-limiting examples of a binding pair includes antibody/antigen, antibody/antibody, antibody/antibody fragment, antibody/antibody receptor, antibody/protein A or protein G, hapten/anti-hapten, sulfhydryl/maleimide, sulfhydryl/haloacetyl derivative, amine/isotriocyanate, amine/succinimidyl ester, amine/sulfonyl halides, biotin/avidin, biotin/streptavidin, folic acid/folate binding protein, receptor/ligand, vitamin B12/intrinsic factor, analogues thereof, derivatives thereof, binding portions thereof, the like or combinations thereof. Non-limiting examples of a binding pair member include an antibody, antibody fragment, reduced antibody, chemically modified antibody, antibody receptor, an antigen, hapten, anti-hapten, a peptide, protein, nucleic acid (e.g., double-stranded DNA (dsDNA), single-stranded DNA (ssDNA), or RNA), a nucleotide, a nucleotide analog or derivative (e.g., bromodeoxyuridine (BrdU)), an alkyl moiety (e.g., methyl moiety on methylated DNA or methylated histone), an alkanoyl moiety (e.g., an acetyl group of an acetylated protein (e.g., an acetylated histone)), an alkanoic acid or alkanoate moiety (e.g., a fatty acid), a glyceryl moiety (e.g., a lipid), a phosphoryl moiety, a glycosyl moiety, a ubiquitin moiety, lectin, aptamer, receptor, ligand, metal ion, avidin, neutravidin, biotin, B12, intrinsic factor, analogues thereof, derivatives thereof, binding portions thereof, the like or combinations thereof. In some embodiments, a member of a binding pair comprises a distinguishable identifier.

In some embodiments the nucleic acids, compositions, formulations, combination products and materials described herein can be included as part of kits, which kits can include one or more of pharmaceutical compositions, nucleic acids, and formulations of the same, combination drugs and products and other materials described herein. In some embodiments the products, compositions, kits, formulations, etc. can come in an amount, package, product format with enough medication to treat a patient for 1 day to 1 year, 1 day to 180 days, 1 day to 120 days, 1 day to 90 days, 1 day to 60 days, 1 day to 30 days, or any day or number of days there between, 1-3 months, 1-2 months, about 3 months, about 2 months, about one month, 3-4 weeks, 3-2 weeks, about 4 weeks, about 3 weeks, about 2 weeks, about 1 week, 1-4 hours, 1-12 hours, or 1-24 hours.

In some embodiments, a kit comprises one or more species-specific ANAGO, such as human ANAGO.

In some embodiments, a kit comprises one or more species-specific ANAGO, such as human ANAGO, and one or more nucleic acid donors.

In some embodiments, a kit comprises one or more species-specific ANAGO, one or more nucleic acid donors, one or more NLS, one or more TAG described herein, or one or more compositions thereof. In some embodiments, a kit comprises one or more human ANAGO, one or more nucleic acid donors, one or more NLS, one or more TAG described herein, or one or more compositions thereof. In some embodiments, a kit comprises one or more human ANAGO encoded with nucleic acid sequence of SEQ ID NO:1, SEQ ID NO:2, SEQ ID NO:3, or SEQ ID NO:4, one or more nucleic acid donors, one or more NLS, one or more TAG described herein, or one or more compositions thereof. In some embodiments, a kit comprises one or more human ANAGO encoded with nucleic acid sequence of SEQ ID NO:1, one or more nucleic acid donors, one or more NLS, one or more TAG described herein, or one or more compositions thereof. In some embodiments, a kit comprises one or more human ANAGO encoded with nucleic acid sequence of SEQ ID NO:2, one or more nucleic acid donors, one or more NLS, one or more TAG described herein, or one or more compositions thereof. In some embodiments, a kit comprises one or more human ANAGO encoded with nucleic acid sequence of SEQ ID NO:3, one or more nucleic acid donors, one or more NLS, one or more TAG described herein, or one or more compositions thereof. In some embodiments, a kit comprises one or more human ANAGO encoded with nucleic acid sequence of SEQ ID NO:4, one or more nucleic acid donors, one or more NLS, one or more TAG described herein, or one or more compositions thereof.

In some embodiments, the kits described herein are used in genome editing in eukaryotic cells. Such a gene editing is for gene therapy in the treatment of disease or conditions.

Some embodiments include kits including pharmaceutical compositions described herein, combination compositions and pharmaceutical formulations thereof, packaged into suitable packaging material. A kit optionally includes a label or packaging insert, or any form of written material, in print or electronic media, including a description of any ANAGO, composition, formulation, or method described herein, or any combination thereof.

### Vehicles for Delivery

The ANAGO described herein is small enough that it can be introduced into eukaryotic cells via numerous mechanisms and tools, and examples of which, but not limited to, are shown below. An ANAGO can be delivered into a mammalian cell directly in a complete protein form or in a form of nucleic acid after the ANAGO is cloned to an expression vector, such as a mammalian expression vector.

### (i) Viral Vector

ANAGO in a form of DNA or RNA can be housed in the capsid of a virus that is known to successfully, or preferentially, or selectively infect a specific cell type under ordinary or laboratory conditions. Injection of the ANAGO-loaded viral particles leads to infection of the target cells and injection of the desired ANAGO into the host cell. The translational machinery and ribosomes proceed to translate the ANAGO into functional Argonaute proteins ready for gene-editing. As the virus spreads, more cells become infected and the quantity of Argonaute protein produced increases proportionally. The Argonaute complex produced can proceed to execute the pre-programmed gene-editing process it was designed for in the target tissues or cells in an organism. This is a preferred method for non-clinical settings, research settings, and for large quantities of cells in vivo or in vitro.

### (ii) Electroporation

Target cells are placed in a dish or plate for in vitro exposure to the ANAGO in a form of RNA. Electric pulses are sent through the plate in order to render the membranes of the target cell porous. ANAGO of interest can flow through these pores into the target cells and reach the translational machinery. The ANAGO and donor nucleic acid are then assembled and ready for editing.

(iii) Lipofection

Nucleic acids can be packaged in spherical molecules composed of lipids. These lipids can fuse with lipophilic cellular membranes and dump the nucleic acid payload into the cell. The component parts can then be assembled into the ANAGO for genome editing.

### (iv) Nucleofection

A variation of many electroporation protocols used to allow nucleic acids to reach the nucleus, can be used to transport ANAGO into the cell/nucleus for transcription and/or translation into the ultimate active protein product.

### (v) Nanoparticle

New formulations of nanoparticles are constantly being developed that can deliver payloads such as the ANAGO into a cell/nucleus. Colloidal gold nanoparticles for example can be used for this purpose safely and efficiently.

### (vi) Microinjection

In larger cell types, direct injection of the ANAGO is possible. This method can be used when only a few cells are targeted.

Based on the literature search, there are 3 characterized prokaryotic Argonaute proteins that have showed DNA-guided DNA interference activities: P. furiosus (Pf-Ago), M. jannaschii (Mj-Ago), and T. thermophilus (Tt-Ago). Additionally, the Ng-Ago is considered as a hypothetical microbial Argonaute (no official and creditable report on its DNA-guided DNA interference activities).

The inventors have done comparison between the nucleic acid sequences of SEQ ID NO:1, SEQ ID NO:2, SEQ ID NO:3, and SEQ ID NO:4 described herein with the naturally occurring microbial Argonaute protein counterparts of P. furiosus (Pf-Ago), M. jannaschii (Mj-Ago), and T. thermophilus (Tt-Ago); and the hypothetical microbial Argonaute Ng-Ago, to obtain the percent identity they share by using the method described below. The inventors performed nucleotide BLAST search online at the below website (https://blast.ncbi.nlm.nih.gov/Blast.cgi?PROGRAM=blastn&PAGE_TYPE=BlastSearch&LINK_LOC=blasthome) on the 4 ANAGO coding sequences (SEQ ID NO:1, SEQ ID NO:2, SEQ ID NO:3, and SEQ ID NO:4). The whole coding sequence of ANAGO is entered as Query sequence. The inventors selected “Nucleotide collection (nr/nt)” under the “Standard databases (nr etc):” and optimized for “Somewhat similar sequences (blastn) under Program Selection. By using this online sequencing searching tool, for example, the inventors found that ANAGO-Pf shares 72% sequence identity with the microbial gene sequence from Pyrococcus furiosus (Pf-AGO). The results are listed below:
1. furiosus (Pf-Ago) (having 771 aa residues, corresponding to 2313 nts) shares 72% sequence identity to the ANAGO-Pf disclosed in the present application;
2. jannaschii (Mj-Ago) (having 715 aa residues, corresponding to 2145 nts) shares 69% sequence identity to the ANAGO-Mj disclosed in the present application; and
3. thermophilus (Tt-Ago) (having 685 aa residues, corresponding to 2055 nts) shares 77% sequence identity to the ANAGO-Tt disclosed in the present application.
4. Ng-Ago (a hypothetical microbial Argonaute, having 887 aa residues, corresponding to 2661 nts) shares 83% sequence identity to the ANAGO-Ng disclosed in the present application.
   Thus, the nucleic acid sequences of SEQ ID NO:1, SEQ ID NO:2, SEQ ID NO:3, and SEQ ID NO:4 described herein shares less than 85% sequence identity from the second nucleic acid sequence comprises a coding region that is capable of encoding the known naturally occurring Argonaute microbial Argonaute protein counterpart that has endonuclease activities in a microorganism. Thus, the ANAGO is not same as its naturally occurring microbial argonaute counterpart. There are differences in structure and other characteristics between ANAGO and its naturally occurring microbial argonaute counterpart. There is also difference in function between the ANAGO and its naturally occurring microbial argonaute counterpart when introduced into mammalian cells.

Some of the embodiments contemplated by the inventors are listed below:

Embodiment 1. A synthetic nucleic acid comprising:
a first nucleic acid sequence comprising about 1000 or more contiguous nucleotides, or portion thereof,
wherein the first nucleic acid sequence encodes an ANAGO that is a polypeptide, capable of editing a target nucleic acid sequence within a eukaryotic cell, wherein the ANAGO is a species-specific to the eukaryote;
wherein the first nucleic acid sequence is modified from a second nucleic acid sequence of a microbial species, and wherein the second nucleic acid sequence comprises a coding region that is capable of encoding a microbial Argonaute protein that has endonuclease activities in a microbial cell; and
wherein the first nucleic acid sequence is modified so that the microbial preferred codons of the second nucleic acid sequence are replaced with codons that have preferential usage in the target eukaryotic species.
   Embodiment 2. The synthetic nucleic acid of embodiment 1, wherein the ANAGO is a human ANAGO, an animal ANAGO, or a plant ANAGO.
   Embodiment 3. The synthetic nucleic acid of embodiment 1, wherein the first nucleic acid sequence comprises at least 70% identity to the nucleic acid sequence of SEQ ID NO:1; SEQ ID NO:2; SEQ ID NO:3, or SEQ ID NO:4, or portion thereof, and wherein the ANAGO is a human ANAGO.
   Embodiment 4. The synthetic nucleic acid of embodiment 1, wherein the first nucleic acid sequence comprises at least 85% identity to the nucleic acid sequence of SEQ ID NO:1; SEQ ID NO:2; SEQ ID NO:3, or SEQ ID NO:4, or portion thereof, and wherein the ANAGO is a human ANAGO.
   Embodiment 5. The synthetic nucleic acid of embodiment 1, wherein the first nucleic acid sequence comprises the nucleic acid sequence of SEQ ID NO:2.
   Embodiment 6. The synthetic nucleic acid of embodiment 1, wherein the first nucleic acid sequence comprises the nucleic acid sequence of SEQ ID NO:3.
   Embodiment 7. The synthetic nucleic acid of embodiment 1, wherein the first nucleic acid sequence comprises the nucleic acid sequence of SEQ ID NO:4.
   Embodiment 8. The synthetic nucleic acid of any one of embodiments 1 to 6, further comprising a promoter operably linked to the coding region.
   Embodiment 9. The synthetic nucleic acid of any one of embodiments 1 to 8, wherein the ANAGO is further attached to a coding sequence of a nuclear localization signal peptide (NLS).
   Embodiment 10. A composition comprising the synthetic nucleic acid of any one of embodiments 1 to 9, and a donor nucleic acid.
   Embodiment 11. The composition of embodiment 10, wherein the donor nucleic acid comprising:
(i) a desired nucleic acid sequence;
(ii) a 5′-flanking sequence; and
(iii) a 3′-flanking sequence,
wherein each of the 5′-flanking sequence and the 3′-flanking sequence independently comprise at least 10 consecutive nucleotides that are at least 90% identical to a target sequence located in the genome of a eukaryotic cell.
   Embodiment 12. The composition of embodiment 11, further comprising one or more of a pharmaceutical acceptable excipient, diluent, additive, or carrier
   Embodiment 13. The composition of any one of embodiments 10 to 12, wherein the synthetic nucleic acid, and the donor nucleic acid are separate nucleic acid fragments.
   Embodiment 14. The composition of any one of embodiments 10 to 13, wherein the synthetic nucleic acid, and the donor nucleic acid are linked via a spacer sequence.
   Embodiment 15. The composition of any one of embodiments 10 to 14, wherein the composition is a pharmaceutical composition comprising a pharmaceutical acceptable excipient.
   Embodiment 16. A kit comprising the synthetic nucleic acid of any one of embodiments 1 to 8, or the composition of any one of embodiments 10 to 15, or the ANAGO of any one of the embodiments 1-13, or a combination thereof.
   Embodiment 17. A method of editing a genome of a eukaryotic cell comprising:
introducing into the cell
(i) a species-specific ANAGO encoded by the first synthetic nucleic acid sequence of any one of embodiments 1 to 16, or an in vitro messenger RNA transcribed by the first synthetic nucleic acid sequence of any one of embodiments 1 to 14; and
(ii) a donor nucleic acid comprising:
   a desired nucleic acid sequence,
   a 5′-flanking sequence, and
   a 3′-flanking sequence,
wherein each of the 5′-flanking sequence and the 3′-flanking sequence are located on opposite sides of the desired nucleic acid sequence and independently comprise at least 10 consecutive nucleotides that are at least 90% identical to a target sequence located in the genome of the eukaryotic cell.
   Embodiment 18. The method of embodiment 17, wherein the eukaryotic cell is a human cell, an animal cell, or a plant cell.
   Embodiment 19. The method of embodiment 17 or 18, wherein the eukaryotic cell is a human cell.
   Embodiment 20. The method of embodiment 17, 18, or 19, wherein the first synthetic nucleic acid sequence comprises at least 70% identity to the nucleic acid sequence of SEQ ID NO:1, SEQ ID NO:2, SEQ ID NO:3, or SEQ ID NO:4, or portion thereof, wherein the ANAGO is a human ANAGO.
   Embodiment 21. The method of embodiment 17, 18, or 19, wherein the first synthetic nucleic acid sequence comprises at least 85% identity to the nucleic acid sequence of SEQ ID NO:1, SEQ ID NO:2, SEQ ID NO:3, or SEQ ID NO:4, or portion thereof, wherein the ANAGO is a human ANAGO.
   Embodiment 22. The method of embodiment 17, 18, or 19, wherein the first synthetic nucleic acid sequence comprises at least 70% identity to the nucleic acid sequence of SEQ ID NO:2, SEQ ID NO:3, or SEQ ID NO:4, or portion thereof, and wherein the ANAGO is a human ANAGO.
   Embodiment 23. The method of embodiment 17, 18, or 19, wherein the first synthetic nucleic acid sequence comprises at least 85% identity to the nucleic acid sequence of SEQ ID NO:2, SEQ ID NO:3, or SEQ ID NO:4, or portion thereof, and wherein the ANAGO is a human ANAGO.
   Embodiment 24. The method of any one of embodiments 20 to 23, wherein the human ANAGO in a protein form or the in vitro transcribed messenger RNA is cloned into a mammalian expression vector before being introduced into the human cell.
   Embodiment 25. The method of embodiment 24, wherein the mammalian expression vector is a plasmid vector, a lentiviral vector, an adeno-associated viral vector, or any viral vector.
   Embodiment 26. The method of any one of embodiments 20 to 23, wherein the human ANAGO is stably expressed after being introduced into the genome of a human cell.
   Embodiment 27. The method of any one of embodiments 17 to 26, wherein the donor nucleic acid is a single-strand molecule.
   Embodiment 28. The method of any one of embodiments 17 to 26, wherein the donor nucleic acid is a double-strand molecule.
   Embodiment 29. The method of any one of embodiments 17 to 28, wherein the 5′-flanking sequence and the 3′-flanking sequence contain 10 to 50 nucleotides in length.
   Embodiment 30. The method of any one of embodiments 17 to 28, the 5′-flanking sequence and the 3′-flanking sequence have 20 to 30 nucleotides in length.
   Embodiment 31. The method of any one of embodiments 17 to 30, wherein each of the 5′-flanking sequence and the 3′-flanking sequence comprise at least 10 nucleotides that are identical to the target sequence.
   Embodiment 32. The method of any one of embodiments 17 to 31, wherein the 5′ and the 3′ flanking sequences are different.
   Embodiment 33. The method of any one of embodiments 17 to 32, wherein the target sequence contains 1 or more nucleotides in length.
   Embodiment 34. The method of embodiment 17, wherein the ANAGO is cloned into a eukaryotic expression vector before the ANAGO is introduced into the cell.
   Embodiment 35. The method of embodiment 17, wherein the desired nucleic acid sequence of the donor nucleic acid comprises a human gene or portion thereof.
   Embodiment 36. The method of any one of embodiments 17 to 35, wherein the target sequence is modified.
   Embodiment 37. The method of embodiment 36, wherein the modification comprises a deletion, an insertion, replacement of one or more nucleotides, or a combination thereof.
   Embodiment 38. The method of embodiment 36, wherein the modification comprises a single nucleotide deletion, a single nucleotide insertion, or a single nucleotide replacement.
   Embodiment 39. The method of any one of embodiments 17 to 38, wherein the editing of the genome of the eukaryotic cell occurs in a homologous sequence-dependent manner.
   Embodiment 40. The method of any one of embodiments 17 to 38, wherein the eukaryotic cell is a human cell, and wherein the editing of the genome of the human cell occurs in a homologous sequence-dependent manner.
   Embodiment 41. The method of any one of embodiments 17 to 40, wherein the ANAGO is introduced into the cell via viral vector, electroporation, lipofection, nucleofection, nanoparticle, or microinjection.
   Embodiment 42. The method of any one of embodiments 17 to 41, wherein a single or multiple donor molecules targeting different sites are introduced into a eukaryotic cell at same time for multiplex genome editing at same time.
   Embodiment 43. The synthetic nucleic acid or the ANAGO of any one of embodiments 1 to 8, the compositions of any one of embodiments 10 to 15, or the kit of embodiment 16 for use in gene therapy or genome editing.
   Embodiment 44. A method of treating a disease, a disorder, or a condition treatable with genome editing in eukaryotic cells comprising, introducing an ANAGO of any one of embodiment 1-43 to a eukaryotic cell.
   Embodiment 45. The method of embodiment 44, wherein genome editing in eukaryotic cells is for chronic myelogenous leukemia; lowering LDL levels in blood stream; or enhancing the effectiveness of immune therapy against tumor cells.
   Embodiment 46. A synthetic nucleic acid comprising:
a first nucleic acid sequence comprising about 1000 or more contiguous nucleotides, or a portion thereof,
wherein the first nucleic acid sequence encodes a human ANAGO that is a polypeptide, capable of editing a target nucleic acid sequence within a human stem cell;
wherein the first nucleic acid sequence is modified from a second nucleic acid sequence of a microbial species, and wherein the second nucleic acid sequence comprises a coding region that is capable of translating a microbial Argonaute protein that has endonuclease activities in a microorganism; and
wherein the first nucleic acid sequence is modified so that the microbial preferred codons of the second nucleic acid sequence are replaced with codons that have preferential usage in a human.
   Embodiment 47. The synthetic nucleic acid of embodiment 46, wherein the first nucleic acid sequence comprises at least 70% identity to the nucleic acid sequence of SEQ ID NO:1; SEQ ID NO:2; SEQ ID NO:3, or SEQ ID NO:4, or a portion thereof.
   Embodiment 48. The synthetic nucleic acid of embodiment 46, wherein the first nucleic acid sequence comprises at least 85% identity to the nucleic acid sequence of SEQ ID NO:1; SEQ ID NO:2; SEQ ID NO:3, or SEQ ID NO:4, or a portion thereof.
   Embodiment 49. The synthetic nucleic acid of embodiment 46, wherein the first nucleic acid sequence comprises the nucleic acid sequence of SEQ ID NO:1.
   Embodiment 50. The synthetic nucleic acid of embodiment 46, wherein the first nucleic acid sequence comprises the nucleic acid sequence of SEQ ID NO:2.
   Embodiment 51. The synthetic nucleic acid of embodiment 46, wherein the first nucleic acid sequence comprises the nucleic acid sequence of SEQ ID NO:3.
   Embodiment 52. The synthetic nucleic acid of embodiment 46, wherein the first nucleic acid sequence comprises the nucleic acid sequence of SEQ ID NO:4.
   Embodiment 53. The synthetic nucleic acid of embodiment 42, 43, 44, 45, 46, 47, 48, 49, 50, 51, or 52, further comprising a promoter operably linked to the coding region.
   Embodiment 54. The synthetic nucleic acid of embodiment 42, 43, 44, 45, 46, 47, 48, 49, 50, 51, 52, or 53, wherein the ANAGO is further attached to a coding sequence of a nuclear localization signal (NLS) peptide.
   Embodiment 55. A composition comprising the synthetic nucleic acid of embodiment 42, 43, 44, 45, 46, 47, 48, 49, 50, 51, 52, 53, or 54, and a donor nucleic acid.
   Embodiment 56. The composition of embodiment 55, wherein the donor nucleic acid comprises:
(i) a desired nucleic acid sequence;
(ii) a 5′-flanking sequence; and
(iii) a 3′-flanking sequence,
wherein the 5′-flanking sequence and the 3′-flanking sequence independently comprise at least 10 consecutive nucleotides that are at least 90% identical to a target sequence located in the genome of a eukaryotic cell.
   Embodiment 57. The composition of embodiment 56, further comprising one or more of a pharmaceutical acceptable excipient, diluent, additive, or carrier.
   Embodiment 58. A method of editing a genome of a human stem cell comprising:
introducing into the human stem cell
(i) a human ANAGO encoded by the first synthetic nucleic acid sequence of embodiment 42, 43, 44, 45, 46, 47, 48, 49, 50, 51, 52, 53, or 54, or an in vitro messenger RNA transcribed by the first synthetic nucleic acid sequence of embodiment 42, 43, 44, 45, 46, 47, 48, 49, 50, 51, 52, 53, or 54; and
(ii) a donor nucleic acid comprising:
   a desired nucleic acid sequence,
   a 5′-flanking sequence, and
   a 3′-flanking sequence,
wherein the 5′-flanking sequence and the 3′-flanking sequence are located on opposite sides of the desired nucleic acid sequence and independently comprise at least 10 consecutive nucleotides that are at least 90% identical to a target sequence located in the genome of the human stem cell.
   Embodiment 59. The method of embodiment 58, wherein the first synthetic nucleic acid sequence comprises at least 70% identity to the nucleic acid sequence of SEQ ID NO:1, SEQ ID NO:2, SEQ ID NO:3, or SEQ ID NO:4, or a portion thereof.
   Embodiment 60. The method of embodiment 58, wherein the first synthetic nucleic acid sequence comprises at least 85% identity to the nucleic acid sequence of SEQ ID NO:1, SEQ ID NO:2, SEQ ID NO:3, or SEQ ID NO:4, or a portion thereof.
   Embodiment 61. The method of embodiment 58, 59, or 60, wherein the donor nucleic acid is a single-strand molecule.
   Embodiment 62. The method of embodiment 58, 59, or 60, wherein the donor nucleic acid is a double-strand molecule.
   Embodiment 63. The method of embodiment 58, 59, 60, 61, or 62, wherein the 5′-flanking sequence and the 3′-flanking sequence each contain 10 nucleotides to 500 nucleotides.
   Embodiment 64. The method of embodiment 58, 59, 60, 61, 62, or 63, wherein each of the 5′-flanking sequence and the 3′-flanking sequence comprise at least 10 nucleotides that are identical to the target sequence.
   Embodiment 65. The method of 58, 59, 60, 61, 62, 63, or 64, wherein the donor molecule comprises a single nucleotide change as compared to the target sequence.
   Embodiment 66. The method of embodiment 58, 59, 60, 61, 62, 63, 64, or 65, wherein genome editing in the human stem cell is for treating chronic myelogenous leukemia; treating cystic fibrosis; lowering LDL levels in blood stream; or use in hematopoietic stem cells (HSCs) therapy to replace defective bone marrow stem cells.
   Embodiment 67. The method of embodiment 58, 59, 60, 61, 62, 63, 64, 65, or 66, further comprising introducing into the human stem cell
(iii) a dominant negative form of human TP53 gene (P53DD) encoded the synthetic nucleic acid sequence (5′-GGATCCATGCCCCCAGGGAGCACTAAGCGAGCACTGCCCAACAACACCAGCTCCTCTCCCCAGCCAAAG AAGAAACCACTGGATGGAGAATATTTCACCCTTCAGATCCGTGGGCGTGAGCGCTTCGAGATGTTCCGA GAGCTGAATGAGGCCTTGGAACTCAAGGATGCCCAGGCTGGGAAGGAGCCAGGGGGGAGCAGGGCTC ACTCCAGCCACCTGAAGTCCAAAAAGGGTCAGTCTACCTCCCGCCATAAAAAACTCATGTTCAAGACAGA AGGGCCTGACTCAGACAAGCTT-3′ (SEQ ID NO: 45)) is expressed by a mammalian expression vector.
   Embodiment 68. The method of embodiment 58, 59, 60, 61, 62, 63, 64, 65, 66, or 67, further comprising introducing into the human stem cell
(iii) a small interference RNA molecule target Human Rad51.
   Embodiment 69. The method of embodiment 67 or 68, wherein the method increases the gene editing efficiency of the target sequence.
   Embodiment 70. A synthetic nucleic acid comprising:
a first nucleic acid sequence comprising about 1000 or more contiguous nucleotides,
wherein the first nucleic acid sequence encodes a codon-Adapted Nuclear Argonaute protein (ANAGO) that is a polypeptide, capable of editing a target nucleic acid sequence within a human cell in the presence of a donor nucleic acid without a guide nucleic acid, wherein the ANAGO is species-specific to the human, wherein the ANAGO is attached to a coding sequence of a nuclear localization signal (NLS) peptide;
wherein the first nucleic acid sequence is produced by modifying a second nucleic acid sequence of a microbial species, and wherein the second nucleic acid sequence comprises a coding region that is capable of encoding a microbial Argonaute protein that has endonuclease activities in a microorganism;
wherein the modifying comprises replacing microbial preferred codons of the second nucleic acid sequence with codons that have preferential usage in the human cell; and
wherein the first nucleic acid sequence comprises at least 85% identity to the nucleic acid sequence of SEQ ID NO:1; SEQ ID NO:2; SEQ ID NO:3; or SEQ ID NO:4.
   Embodiment 71. The synthetic nucleic acid of embodiment 70, wherein the human cell is a human stem cell.
   Embodiment 72. A composition comprising the synthetic nucleic acid of embodiment 70 or 71, and a donor nucleic acid.
   Embodiment 73. The composition of embodiment 72, wherein the donor nucleic acid comprising:
(i) a desired nucleic acid sequence to be introduced into a target sequence, wherein introducing the desired nucleic acid sequence is induced by an ANAGO;
(ii) a 5′-flanking sequence; and
(iii) a 3′-flanking sequence,
wherein the 5′-flanking sequence and the 3′-flanking sequence independently comprise at least 10 consecutive nucleotides that are at least 90% identical to the target sequence located in the genome of a human cell.
   Embodiment 74. A composition comprising the synthetic nucleic acid of embodiment 70,71, 72, or 73, and one or more of a pharmaceutical acceptable excipient, diluent, additive, or carrier.
   Embodiment 75. A method of editing a genome of a human cell comprising:
introducing into the cell
(i) a species-specific ANAGO encoded by the first nucleic acid sequence of embodiment 70 or 71, or an in vitro messenger RNA transcribed by the first nucleic acid sequence of embodiment 70 or 71; and
(ii) a donor nucleic acid comprising:
   a desired nucleic acid sequence to be introduced into a target sequence, wherein introducing the desired nucleic acid sequence is induced by an ANAGO,
   a 5′-flanking sequence, and
   a 3′-flanking sequence,
wherein the 5′-flanking sequence and the 3′-flanking sequence are located on opposite sides of the desired nucleic acid sequence and independently comprise at least 10 consecutive nucleotides that are at least 90% identical to a target sequence located in the genome of the human cell.
   Embodiment 76. The method of embodiment 75, wherein the human cell is a human stem cell.
   Embodiment 77. The method of embodiment 75 or 76, wherein genome editing in the human cell is for treating chronic myelogenous leukemia; treating cystic fibrosis; lowering LDL levels in blood stream; enhancing the effectiveness of immune therapy against tumor cells in a human being; or use in hematopoietic stem cells (HSCs) therapy to replace defective bone marrow stem cells.

### Examples

### A. Construction of Human ANAGO

The ability to introduce a nucleic acid (e.g., a gene, heterologous DNA or modified nucleic acid) into a genome of an organism at a specific targeted locus is a powerful tool for therapeutic and research purposes. The user-friendly CRISPR-Cas9 is very efficient in making mutations via non-homologous end joining (NHEJ) in human cancer cell lines such as 293T cells. However, it can mediate homologous recombination (HR) only at a much low efficiency (2-5%) in 293T cells and even lower in other biologically relevant cells such as human induced pluripotent stem cells (iPSCs). Gao et al. in a retracted publication (Gao F, et al., (2016) Nat. Biotechnol. 34(7):768-73) reported that it is feasible to achieve genome editing in human cells by using Natronobacterium gregoryi Argonaute (NgAgo) with a guide DNA oligo. However, multiple labs have failed to reproduce this phenomenon claimed by Gao et al. thus far, which led to the withdrawal of this publication later by Gao et al. Gao et al. use the sequence of the NgAgo in their publication.

To investigate whether the ANAGO described herein could be used as a novel and practical gene editing tool in mammalian cells, we reasoned that it is plausible for microbial Argonaute proteins to mediate gene editing in human cells, but at a very low efficiency to be detectable. Particularly, since most of reported DNA targeting Argonaute proteins are the products of either bacterial or archaeal microorganisms, their preference of codon usage is significantly different from the ones used in the translation machinery of human cells. It is known that codon usage is a rate-limiting factor for efficient translation and proper folding of a protein in an organism-specific manner. Therefore, we reengineered and adapted the DNA coding sequences of microbial Argonaute proteins that have DNA endonuclease activities in microbial cells, such as NgAgo, PfAgo, TtAgo and MjAgo, with the codons that are most frequently used in human cells (as described above), to generate humanized Argonaute variants, that comprises, conserves, and/or retains an ability to edit a target nucleic acid sequence within a human cell when expressed in the human cell. We name this type of reengineered and adapted Argonaute variant “ANAGO”. ANAGO is species-specific. When ANAGO comprises the codons that are preferentially used in human cells, we call it human ANAGO.

In order to introduce the ANAGO into the nucleus of mammalian cells, we fused a SV40 nuclear localization signal peptide sequence (NLS) to the N-terminus of the protein in ANAGO. A small Human influenza hemagglutinin (HA) tag was also included for protein detection purposes. The DNA coding sequences of the human ANAGO, attached to a NLS and a HA tag and derived from (a) NgAgo, (b) PfAgo, (c) TtAgo, and (d) MjAgo, are shown below, wherein the HA epitope tag sequence is framed; and the SV40 nuclear localization signal (NLS) sequence is underlined.

(a) Nucleic Acid Sequence of SEQ ID NO: 1-DNA sequence of a human ANAGO of NLS-NgAgo
ORF
ATGGTGCCAAAAAAGAAGAGAAAGGTAGCCACCGTGATCGACCTGGACTCCACCACCACCGCCGACGA
GCTGACCTCCGGCCACACCTACGACATCTCCGTGACCCTGACCGGCGTGTACGACAACACCGACGAGCA
GCACCCCCGGATGTCCCTGGCCTTCGAGCAGGACAACGGCGAGCGGCGGTACATCACCCTGTGGAAGA
ACACCACCCCCAAGGACGTGTTCACCTACGACTACGCCACCGGCTCCACCTACATCTTCACCAACATCGAC
TACGAGGTGAAGGACGGCTACGAGAACCTGACCGCCACCTACCAGACCACCGTGGAGAACGCCACCGC
CCAGGAGGTGGGCACCACCGACGAGGACGAGACCTTCGCCGGCGGCGAGCCCCTGGACCACCACCTGG
ACGACGCCCTGAACGAGACCCCCGACGACGCCGAGACCGAGTCCGACTCCGGCCACGTGATGACCTCCT
TCGCCTCCCGGGACCAGCTGCCCGAGTGGACCCTGCACACCTACACCCTGACCGCCACCGACGGCGCCA
AGACCGACACCGAGTACGCCCGGCGGACCCTGGCCTACACCGTGCGGCAGGAGCTGTACACCGACCAC
GACGCCGCCCCCGTGGCCACCGACGGCCTGATGCTGCTGACCCCCGAGCCCCTGGGCGAGACCCCCCTG
GACCTGGACTGCGGCGTGCGGGTGGAGGCCGACGAGACCCGGACCCTGGACTACACCACCGCCAAGGA
CCGGCTGCTGGCCCGGGAGCTGGTGGAGGAGGGCCTGAAGCGGTCCCTGTGGGACGACTACCTGGTGC
GGGGCATCGACGAGGTGCTGTCCAAGGAGCCCGTGCTGACCTGCGACGAGTTCGACCTGCACGAGCGG
TACGACCTGTCCGTGGAGGTGGGCCACTCCGGCCGGGCCTACCTGCACATCAACTTCCGGCACCGGTTC
GTGCCCAAGCTGACCCTGGCCGACATCGACGACGACAACATCTACCCCGGCCTGCGGGTGAAGACCACC
TACCGGCCCCGGCGGGGCCACATCGTGTGGGGCCTGCGGGACGAGTGCGCCACCGACTCCCTGAACAC
CCTGGGCAACCAGTCCGTGGTGGCCTACCACCGGAACAACCAGACCCCCATCAACACCGACCTGCTGGA
CGCCATCGAGGCCGCCGACCGGCGGGTGGTGGAGACCCGGCGGCAGGGCCACGGCGACGACGCCGTG
TCCTTCCCCCAGGAGCTGCTGGCCGTGGAGCCCAACACCCACCAGATCAAGCAGTTCGCCTCCGACGGCT
TCCACCAGCAGGCCCGGTCCAAGACCCGGCTGTCCGCCTCCCGGTGCTCCGAGAAGGCCCAGGCCTTCG
CCGAGCGGCTGGACCCCGTGCGGCTGAACGGCTCCACCGTGGAGTTCTCCTCCGAGTTCTTCACCGGCA
ACAACGAGCAGCAGCTGCGGCTGCTGTACGAGAACGGCGAGTCCGTGCTGACCTTCCGGGACGGCGCC
CGGGGCGCCCACCCCGACGAGACCTTCTCCAAGGGCATCGTGAACCCCCCCGAGTCCTTCGAGGTGGCC
GTGGTGCTGCCCGAGCAGCAGGCCGACACCTGCAAGGCCCAGTGGGACACCATGGCCGACCTGCTGAA
CCAGGCCGGCGCCCCCCCCACCCGGTCCGAGACCGTGCAGTACGACGCCTTCTCCTCCCCCGAGTCCATC
TCCCTGAACGTGGCCGGCGCCATCGACCCCTCCGAGGTGGACGCCGCCTTCGTGGTGCTGCCCCCCGAC
CAGGAGGGCTTCGCCGACCTGGCCTCCCCCACCGAGACCTACGACGAGCTGAAGAAGGCCCTGGCCAAC
ATGGGCATCTACTCCCAGATGGCCTACTTCGACCGGTTCCGGGACGCCAAGATCTTCTACACCCGGAACG
TGGCCCTGGGCCTGCTGGCCGCCGCCGGCGGCGTGGCCTTCACCACCGAGCACGCCATGCCCGGCGACG
CCGACATGTTCATCGGCATCGACGTGTCCCGGTCCTACCCCGAGGACGGCGCCTCCGGCCAGATCAACAT
CGCCGCCACCGCCACCGCCGTGTACAAGGACGGCACCATCCTGGGCCACTCCTCCACCCGGCCCCAGCT
GGGCGAGAAGCTGCAGTCCACCGACGTGCGGGACATCATGAAGAACGCCATCCTGGGCTACCAGCAGG
TGACCGGCGAGTCCCCCACCCACATCGTGATCCACCGGGACGGCTTCATGAACGAGGACCTGGACCCCG
CCACCGAGTTCCTGAACGAGCAGGGCGTGGAGTACGACATCGTGGAGATCCGGAAGCAGCCCCAGACC
CGGCTGCTGGCCGTGTCCGACGTGCAGTACGACACCCCCGTGAAGTCCATCGCCGCCATCAACCAGAAC
GAGCCCCGGGCCACCGTGGCCACCTTCGGCGCCCCCGAGTACCTGGCCACCCGGGACGGCGGCGGCCT
GCCCCGGCCCATCCAGATCGAGCGGGTGGCCGGCGAGACCGACATCGAGACCCTGACCCGGCAGGTGT
ACCTGCTGTCCCAGTCCCACATCCAGGTGCACAACTCCACCGCCCGGCTGCCCATCACCACCGCCTACGC
CGACCAGGCCTCCACCCACGCCACCAAGGGCTACCTGGTGCAGACCGGCGCCTTCGAGTCCAACGTGGG
CTTCCTG
(b) Nucleic Acid Sequence of SEQ ID NO: 2-DNA sequence of a human ANAGO of HA-NLS-PfAgo
ORF

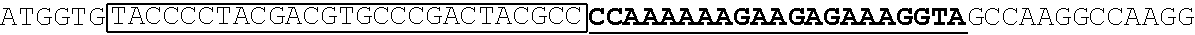

TGGTGATCAACCTGGTGAAGATCAACAAGAAGATCATCCCCGACAAGATCTACGTGTACAGACTGTTCA
ACGACCCCGAGGAGGAGCTGCAGAAGGAGGGCTACAGCATCTACAGACTGGCCTACGAGAACGTGGGC
ATCGTGATCGACCCCGAGAACCTGATCATCGCCACCACCAAGGAGCTGGAGTACGAGGGCGAGTTCATC
CCCGAGGGCGAGATCAGCTTCAGCGAGCTGAGAAACGACTACCAGAGCAAGCTGGTGCTGAGACTGCT
GAAGGAGAACGGCATCGGCGAGTACGAGCTGAGCAAGCTGCTGAGAAAGTTCAGAAAGCCCAAGACCT
TCGGCGACTACAAGGTGATCCCCAGCGTGGAGATGAGCGTGATCAAGCACGACGAGGACTTCTACCTG
GTGATCCACATCATCCACCAGATCCAGAGCATGAAGACCCTGTGGGAGCTGGTGAACAAGGACCCCAAG
GAGCTGGAGGAGTTCCTGATGACCCACAAGGAGAACCTGATGCTGAAGGACATCGCCAGCCCCCTGAA
GACCGTGTACAAGCCCTGCTTCGAGGAGTACACCAAGAAGCCCAAGCTGGACCACAACCAGGAGATCGT
GAAGTACTGGTACAACTACCACATCGAGAGATACTGGAACACCCCCGAGGCCAAGCTGGAGTTCTACAG
AAAGTTCGGCCAGGTGGACCTGAAGCAGCCCGCCATCCTGGCCAAGTTCGCCAGCAAGATCAAGAAGA
ACAAGAACTACAAGATCTACCTGCTGCCCCAGCTGGTGGTGCCCACCTACAACGCCGAGCAGCTGGAGA
GCGACGTGGCCAAGGAGATCCTGGAGTACACCAAGCTGATGCCCGAGGAGAGAAAGGAGCTGCTGGA
GAACATCCTGGCCGAGGTGGACAGCGACATCATCGACAAGAGCCTGAGCGAGATCGAGGTGGAGAAG
ATCGCCCAGGAGCTGGAGAACAAGATCAGAGTGAGAGACGACAAGGGCAACAGCGTGCCCATCAGCCA
GCTGAACGTGCAGAAGAGCCAGCTGCTGCTGTGGACCAACTACAGCAGAAAGTACCCCGTGATCCTGCC
CTACGAGGTGCCCGAGAAGTTCAGAAAGATCAGAGAGATCCCCATGTTCATCATCCTGGACAGCGGCCT
GCTGGCCGACATCCAGAACTTCGCCACCAACGAGTTCAGAGAGCTGGTGAAGAGCATGTACTACAGCCT
GGCCAAGAAGTACAACAGCCTGGCCAAGAAGGCCAGAAGCACCAACGAGATCGGCCTGCCCTTCCTGG
ACTTCAGAGGCAAGGAGAAGGTGATCACCGAGGACCTGAACAGCGACAAGGGCATCATCGAGGTGGTG
GAGCAGGTGAGCAGCTTCATGAAGGGCAAGGAGCTGGGCCTGGCCTTCATCGCCGCCAGAAACAAGCT
GAGCAGCGAGAAGTTCGAGGAGATCAAGAGAAGACTGTTCAACCTGAACGTGATCAGCCAGGTGGTGA
ACGAGGACACCCTGAAGAACAAGAGAGACAAGTACGACAGAAACAGACTGGACCTGTTCGTGAGACAC
AACCTGCTGTTCCAGGTGCTGAGCAAGCTGGGCGTGAAGTACTACGTGCTGGACTACAGATTCAACTAC
GACTACATCATCGGCATCGACGTGGCCCCCATGAAGAGAAGCGAGGGCTACATCGGCGGCAGCGCCGT
GATGTTCGACAGCCAGGGCTACATCAGAAAGATCGTGCCCATCAAGATCGGCGAGCAGAGAGGCGAGA
GCGTGGACATGAACGAGTTCTTCAAGGAGATGGTGGACAAGTTCAAGGAGTTCAACATCAAGCTGGAC
AACAAGAAGATCCTGCTGCTGAGAGACGGCAGAATCACCAACAACGAGGAGGAGGGCCTGAAGTACAT
CAGCGAGATGTTCGACATCGAGGTGGTGACCATGGACGTGATCAAGAACCACCCCGTGAGAGCCTTCGC
CAACATGAAGATGTACTTCAACCTGGGCGGCGCCATCTACCTGATCCCCCACAAGCTGAAGCAGGCCAA
GGGCACCCCCATCCCCATCAAGCTGGCCAAGAAGAGAATCATCAAGAACGGCAAGGTGGAGAAGCAGA
GCATCACCAGACAGGACGTGCTGGACATCTTCATCCTGACCAGACTGAACTACGGCAGCATCAGCGCCG
ACATGAGACTGCCCGCCCCCGTGCACTACGCCCACAAGTTCGCCAACGCCATCAGAAACGAGTGGAAGA
TCAAGGAGGAGTTCCTGGCCGAGGGCTTCCTGTACTTCGTG
(c) Nucleic Acid Sequence of SEQ ID NO: 3-DNA sequence of a human ANAGO of HA-NLS-TtAgo
ORF

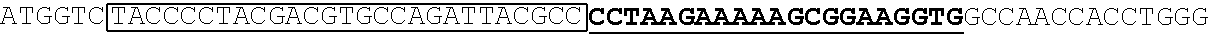

CAAGACAGAGGTGTTCCTGAACAGATTCGCCCTGCGGCCTCTGAACCCTGAGGAACTCAGACCTTGGCG
GCTGGAAGTGGTGCTGGATCCTCCACCTGGACGCGAGGAAGTGTATCCTCTGCTGGCTCAAGTGGCTCG
GAGAGCTGGCGGAGTGACAGTTAGAATGGGAGATGGCCTGGCCAGCTGGTCCCCACCTGAAGTTCTTG
TGCTGGAAGGCACCCTGGCCAGAATGGGCCAGACATACGCCTACCGGCTGTACCCCAAAGGCAGAAGG
CCTCTGGATCCCAAGGATCCCGGCGAGAGATCTGTGCTGTCTGCCCTGGCTAGACGGCTGCTGCAAGAG
AGACTGAGAAGGCTCGAAGGCGTGTGGGTGGAAGGACTGGCCGTGTACAGAAGAGAGCACGCCAGAG
GACCTGGCTGGCGAGTTCTTGGCGGAGCTGTTCTGGATCTGTGGGTGTCAGATAGCGGCGCCTTTCTGC
TGGAAGTCGACCCCGCCTATAGAATCCTGTGCGAGATGAGCCTGGAAGCTTGGCTGGCTCAGGGACACC
CTCTGCCTAAAAGAGTGCGGAACGCCTACGACAGACGGACCTGGGAACTGCTGAGACTGGGCGAAGAG
GACCCCAAAGAACTTCCTCTGCCTGGCGGACTGAGCCTGCTGGATTACCACGCCTCTAAGGGCAGACTG
CAGGGCAGAGAAGGTGGAAGAGTGGCCTGGGTTGCCGATCCTAAGGACCCCAGAAAGCCCATTCCTCA
CCTGACAGGACTGCTGGTGCCTGTGCTGACCCTGGAAGATCTGCACGAGGAAGAGGGATCTCTGGCCCT
GTCTCTGCCTTGGGAAGAGAGAAGAAGGCGGACCAGAGAGATCGCCAGCTGGATCGGAAGAAGGCTT
GGCCTGGGAACACCTGAGGCTGTTAGAGCCCAGGCCTACAGACTGAGCATCCCCAAGCTGATGGGCCG
CAGAGCCGTGTCTAAACCTGCCGATGCTCTGAGAGTGGGCTTCTACAGAGCCCAAGAGACAGCCCTGGC
TCTGCTCAGACTTGATGGCGCTCAAGGCTGGCCCGAGTTTCTGAGAAGGGCTCTGCTGAGAGCCTTTGG
AGCCTCTGGCGCTTCTCTGAGACTGCACACACTGCACGCCCATCCTTCTCAGGGCCTCGCCTTTAGAGAG
GCTCTGAGAAAGGCCAAAGAAGAGGGCGTTCAGGCCGTGCTGGTTCTGACACCTCCTATGGCATGGGA
AGATCGGAACCGGCTGAAAGCCCTGCTGCTCAGAGAGGGACTGCCTAGCCAGATCCTGAACGTGCCCCT
GAGAGAAGAGGAACGGCACAGATGGGAGAATGCCCTGCTGGGCCTGCTGGCCAAAGCTGGACTTCAAG
TGGTTGCCCTGTCCGGCGCCTATCCTGCTGAACTGGCTGTGGGATTTGACGCTGGCGGCAGAGAGAGCT
TCAGATTTGGAGGCGCTGCTTGTGCCGTTGGCGGAGATGGTGGACATCTGCTGTGGACACTGCCTGAAG
CTCAGGCCGGCGAAAGAATCCCTCAAGAGGTCGTGTGGGACCTGCTCGAAGAAACCCTGTGGGCCTTCA
GAAGAAAGGCCGGCAGGCTGCCTTCAAGAGTGCTGCTCCTGAGAGATGGCAGAGTGCCCCAGGATGAG
TTTGCCCTGGCACTGGAAGCCCTGGCAAGAGAGGGAATTGCCTACGACCTGGTGTCCGTGCGGAAATCT
GGTGGCGGAAGAGTGTACCCCGTGCAAGGCAGACTGGCCGATGGACTGTATGTGCCTCTGGAAGATAA
GACCTTCCTGCTGCTGACCGTGCACCGGGACTTTAGAGGCACACCCAGACCTCTGAAGCTGGTGCATGA
AGCCGGCGACACACCTCTCGAAGCTCTGGCCCACCAGATCTTTCACCTGACCAGACTGTACCCCGCCAGC
GGCTTTGCCTTTCCTAGACTGCCTGCTCCTCTGCACCTGGCCGACAGACTGGTCAAAGAAGTGGGCCGCC
TGGGCATCAGACACCTGAAAGAGGTGGACCGCGAGAAGCTGTTCTTCGTG
(d) Nucleic Acid Sequence of SEQ ID NO:4 - DNA sequence of a human ANAGO of HA-NLS-MjAgo
ORF

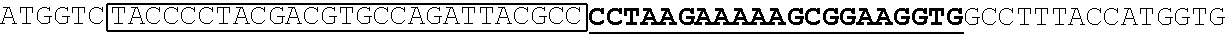

CTGAACAAAGTGACCTACAAGATCAACGCCTATAAGATCAAAGAGGAATTCATCCCCAAAGAGGTGCAC
TTCTACCGGATCAAGAGCTTCGTGAACGAGGCCTTCAACTTCTACAGATTCGTGAACTTCTACGGCGGCA
TGATCATCAACAAGAAAGACAAGTCCTTCGTGCTGCCCTACAAGGTGGACAACAAGGTGCTGAAGTACA
AGGACGGCAACAACGAGATCCCCATCGACATCGAGTACATCAAGAGCCTGAAGCTCGAGTACGTGAAG
CCCGAGATCGCCGAGAAGCTTGTGCGGGGCTATCTGAAGTCCGTGCACAAGATCGAGCCCGAGCTGAG
CCGGATCATCAAGAACATCCGGAAGCACAAGGTGGTGGAAAACATCAAGGTGGAAAGCTACTGCGAGT
ACGAAGTGAAGAAGCACGACGGCGACTACTACCTGATCCTGAACTTCAGACACACCGCCAGCATCACCA
AGCACCTGTGGGACTTCGTGAATAGAGACAAGGCCCTGCTGGAAGAGTACGTGGGCAAGAAGATCATC
TTCAAGCCCAATCCTAAAGTGCGGTACACCATCAGCCTGGTGGACGCCCCAAATCCTCAGAAAATCGAG
GAAATCATGAGCCACATCATCAAGTACTACAAGTGGAGCGAGGACATGGTCAAGAGCACCTTCGGCGA
GATCGACTACAACCAGCCTATCATGTACTGCGAGGAAATTCTGGAACCCTTCGCACCCCAGTTCTGCAAC
CTGGTGTTCTACATGGACGAGCTGGACAGCTACATCCTGAAAGAGCTGCAGAGCTACTGGCGGCTGAGC
AACGAGAACAAGGGCAAGATCATTAACGAGATTGCCAAGAAACTGCGGTTCATCGACAACACGCCCAAA
GAACTGGAATTCATGAAGTTCAACAACACCCCGCTGCTGGTCAAGGACGTGAACAAGAACCCCACCAAG
ATCTACAGCACCAACACACTGTTCACCTGGATCTACAATCAGAACGCCAAAATCTACCTGCCTTACGACGT
CCCCGAGATCATCCGGAACAAGAATCTGCTGACCTACATCCTCATCGACGAAGAGATCAAGGATGAGCT
GAAGGCCATCAAGGACAAAGTCAACAAGATGTTCCGCAACTACAACAAGATCGCCAACAAGACCGAGCT
GCCCAAGTTCAACTACGCCAACCGGTGGAAGTACTTTAGCACCGACGACATCCGGGGCATCATCAAAGA
GATTAAGAGCGAGTTCAACGACGAGATCTGCTTCGCCCTGATCATCGGCAAAGAGAAGTATAAGGACAA
CGATTACTACGAGATCCTCAAGAAGCAGCTGTTCGACCTGAAGATTATCAGCCAGAACATCCTGTGGGA
GAACTGGCGGAAGGACGACAAGGGCTACATGACCAACAACCTGCTGATCCAGATCATGGGCAAGCTGG
GCATCAAGTATTTCATCCTGGACAGCAAGACCCCGTACGACTACATCATGGGCCTCGATACAGGCCTGG
GCATCTTCGGCAATCACAGAGTCGGCGGCTGTACCGTGGTGTACGATAGCGAGGGAAAGATCCGGCGG
ATCCAGCCAATCGAGACACCAGCTCCAGGCGAGAGACTGCATCTGCCCTACGTGATCGAGTACCTGGAA
AACAAGGCCAACATCGACATGGAAAACAAAAACATCCTGTTCCTCCGCGACGGCTTCATCCAGAACAGC
GAGCGGAACGATCTGAAAGAGATCAGCAAAGAGCTGAACAGCAATATCGAAGTGATCTCTATTCGGAA
GAACAACAAGTACAAAGTGTTCACCAGCGACTACAGGATCGGCAGCGTGTTCGGCAACGACGGCATCTT
CCTGCCTCACAAGACCCCTTTCGGCAGCAACCCTGTGAAGCTGAGCACCTGGCTGAGATTCAACTGCGGC
AACGAGGAAGGCCTGAAAATCAACGAGAGCATCATGCAGCTGCTGTACGATCTGACCAAGATGAACTAC
AGCGCCCTGTACGGCGAGGGCAGATACCTGAGAATCCCCGCTCCTATCCACTACGCCGACAAGTTCGTG
AAGGCCCTGGGCAAAAACTGGAAGATCGACGAGGAACTGCTGAAGCACGGCTTTCTGTACTTCATC

### B. ANAGO Induced Homologous Recombination Directed Genome Editing in Eukaryotes

### Materials and Methods

Constructs of ANAGO expression cassette: The ANAGO encoded with the humanized DNA sequence of NLS-NgAgo ORF, or HA-NLS-PfAgo ORF, or HA-NLS-TtAgo ORF, or HA-NLS-MjAgo ORF was chemically synthesized and fused to the P2A-YFP cDNA cassette. The expression of fusion open reading frame was driven by EFlalpha promoter in a mammalian expression vector (SynBio Tech, New Jersey).

Cell culture and transfection. HEK293 (ATCC catalog #CRL-1573) cells were maintained in a DMEM high glucose medium supplemented with 10% fetal bovine serum and 100 U/ml penicillin and 100 ug/ml streptomycin. Cells were seeded into 12-well plates one day before transfection. Cells were transfected at about 50% confluence using LIPOFECTAMINE™3000 (Thermo Scientific). Specifically, HEK293 were transfected with 1000 ng of a human ANAGO-expressing plasmid plus certain amount of donor DNA fragment that is dependent on its size (for examples, 240 ng of 1.8 kb PCSK9-mCherry knock-in fragment or 200 nM of single-stranded oligonucleotide PCSK9 R104C-V114A 70mer for PCSK9 gene in example 1, see FIG. 1a). Cells were harvested for genomic DNA extraction 72 hours post transfection. The donor fragment directed genomic editing event was identified and confirmed by PCR. Subsequently, the genomic PCR products are sequenced and analyzed.

We have used a few human ANAGO described herein to induce homologous recombination directed genome editing in human cells successfully. We have discovered that the use of the human ANAGO to induce genome editing in human cells does not require the presence of a guide DNA molecule. ANAGO induced genome editing is highly precise and produces very low numbers of associated indel events in the target site in all cases tested. Selected examples of using an ANAGO-induced sequence editing (AISE) technology to modify genes in human cells are shown below.

### Example 1. Human PCSK9

Proprotein convertase subtilisin/kexin type9 (PCSK9), acts in lipoprotein homeostasis. It binds and removes low-density lipoprotein receptors (LDLR). If not binding PCSK9, LDLR will return to the cell surface and can continue to remove LDL-particles from the bloodstream. Agents which block PCSK9 can lower LDL particle concentrations. Significantly, individuals with complete naturally occurring heterozygous loss-of-function (LOF) mutations of PCSK9 gene have a near 88% reduced risk of developing cardiovascular complications over a 15-year follow-up period [Cohen J C, Boerwinkle E, et al, 2006, NEJM 354:1264-1272]. Moreover, carriers of LOF mutations such as deltaR97+Y142X, C679X, and R104C+V114A were found to lack PCSK9 expression. These individuals with natural occurring LOF mutations have very low LDL levels in their blood stream without association with any obvious deleterious effects. This supports the clinical utility of introducing LOF mutations to PCSK9 through gene editing to treat hypercholesterolemia.

To test if ANAGO induced sequence editing (AISE) technology described herein could be used to introduce specific PCSK9 LOF mutations in mammalian cells, we first constructed a donor molecule via fusion PCR, which contains the mCherry coding sequence flanked by the homology regions to the exon 1 and intron 1 sequence of PCSK9 gene on the left and right sides respectively. The homology directed replacement (HDR) of the mCherry fragment removes the entire exon 1 as well as 88 bps of the intron 1 sequence of PCSK9 (FIG. 1a). The protein expression constructs of human ANAGO (NLS-NgAgo ORF, HA-NLS-PfAgo ORF, and HA-NLS-TtAgo ORF) and the donor molecule were co-transfected into HEK293 either with or without a guide oligo PCSK9-GD8S (5′-TGGGTCCCGCGGGCGCCCGTGCGC-3′ (SEQ ID NO: 7)) that targets exon 1 by using LIPOFECTAMINE™ 3000. The cellular genomic DNA was harvested at 72 hours post transfection. PCR of genomic DNA samples was carried out to identify the mCherry knock-in event by pairing a donor mCherry specific primer (mCherry-For: 5′-CCTTTCCCACAACGAGGACT-3′ (SEQ ID NO: 8)) with a flanking region of PCSK9 specific primer (PCSK9-in1-R3: 5′-CGAGAATACCTCCGCCCCTT-3′ (SEQ ID NO: 9)), which is located outside of the right homology arm (FIG. 1b). The genomic PCR product was only detectable from the cell samples that were transfected with an ANAGO expression construct and the 1.8 kb of mCherry KI donor fragment either with or without the guide oligo, but not in the absence of the donor fragment (FIG. 1b).

In a separate experimental setting, instead of using an over 500 bps long and double stranded homologous sequence as a flanking arm to facilitate HDR, we tested whether a short and single-stranded oligodeoxynucleotide, PCSK9 R104C-V114A 70mer (5′-CCTGCAGGCCCAGGCTGCC [symbol] GCCGGGGATACCTCACCAAGATCCTGCATG [symbol] CTTCCATGGCCTTCTTCCT-3′ (SEQ ID NO: 5)), which mimics R104C (CGC>TGC)+V114A (GTC>GCC) LOF mutant allele sequence of human PCSK9 gene, could be used as a donor template. The mutated bases are located at the 20th and 40th positions of the 70mer respectively. To estimate the relative frequency of sequence exchanging in the transfected cells, a pair of primers (PCSK9 F9: 5′-GCTTTTTGGTCCGCATTTGG-3′ (SEQ ID NO: 10) and PCSK9 R9: 5′-GGCTCTACCCCTAGCTGTCT-3′ (SEQ ID NO: 11)) located outside of the donor target region was used to generate the genomic PCR product for Sanger sequencing analysis. The results indicated that about 7.1% of genomic PCR products contained the intended R104C+V114A mutation incorporated into the PCSK9 allele in HEK293 cells (FIG. 1c). This result also validates the notion that a short homologous region of 19 bases on each side of modified bases is long enough to generate a desired HDR. In addition, we tested another single-stranded oligo 89mer, PCSK9 Y142X-E144X_donor89_Bgl2 (5′-TCTCTGGCTTCTGCAGGCCTTGAAGTTGCCCCATGTCGACT [symbol] AGGAGGACTCCTCTGTCTTTGCCC AGAGCATCCCGTGGAACC-3′ (SEQ ID NO: 12)), which introduces LOF mutations Y142X (TAC>TAG) and E144X (GAG>TAG) as well as a Bgl2 site (underlined bases) into the exon 3 sequence of PCSK9 gene (FIG. 1a). The Y142X LOF mutation of PCSK9 was found naturally in an African American woman, who has no detectable PCSK9 expression but apparently in good health. Her circulating LDL level is at 0.36 mM, which is about 8-fold lower than normal (Zhao Z, et al., Am i Hum Genet. 2006; 79:514-523). A pair of specific PCR primers (PCSK9 F8 primer 5′-CCGTGTTGCAGGGATATGGG-3′ (SEQ ID NO: 13) and PCSK9 R8 primer 5′-CATTTGTGGGGCAACAGGAAG-3′ (SEQ ID NO: 14)) located in the flanking introns were used to generate a 541 bp PCR amplicon for sequencing analysis. In this case, 3% of amplicon were the HDR product, meanwhile, the non-HDR indel incidence was nearly undetectable (FIG. 1d). These studies suggest that AISE technology could be used to specifically inactivate PCSK9 gene and thus to reduce the circulating LDL level in patients with hypercholesterolemia.

### Example 2. Human ABL1

The Philadelphia (Ph) translocation t(9; 22)(q34; q11) leads ABL1 to fuse with BCR. The resulting BCR-ABL fusion gene is the main reason causing chronic myelogenous leukemia (CML) and Ph+ acute lymphoblastic leukemia (ALL). BCR-ABL encodes a constitutively active cytoplasmic tyrosine kinase that is necessary and sufficient to induce and maintain leukemic transformation. The age-adjusted incidence is 1.6 per 100,000 population members. Small molecular inhibitors have been developed to inhibit BCR-ABL activity. However, drug resistance often occurs in many patients receiving treatment.

Since the main cause of disease is the hyper kinase activity of the fusion protein, an insertion of a premature stop codon in the front of the coding region of kinase domain should result in a truncated product that lacks the kinase domain and thereby inactivates the BCR-ABL fusion gene. A precise gene editing with an AISE approach described herein to inactivate the fusion gene could be done ex vivo with a patient's bone marrow hematopoietic stem cells (HSCs). This approach may cure the disease permanently for CML and Ph+ ALL patients.

BCR-ABLex6stop-H3, a single-stranded oligodeoxynucleotides 70mer (5′-GGCAGGGGTCTGCACCCGGGAGCCCCCGTTCTAAGCTTTCACTGAGTTCATGACCTACGGGAACCTCCTG-3′ (SEQ ID NO: 6)), which changed ABL1 residue Tyr312 codon TAT to a stop codon TAA as well as introduced a Hind III site, which was designed and synthesized. The engineered site is flanked by 32 nucleotides of short homology sequence on each side of the 70mer donor oligo (FIG. 2a). To test whether the 70mer of single-stranded oligo harboring 5 altered bases could be used as a donor molecule to introduce the compound stop codon/Hind III site into the precise target site of ABL1 exon 6 of human genome, we co-transfected a human ANAGO expression construct together with the donor oligo BCR-ABLex6stop-H3 70mer into HEK293 cells. The genomic DNA of transfected cells was harvested at 96 hours post transfection. The genomic region containing the target site was amplified by PCR with a pair of specific primers (ABL-F1: 5′-GCGTCTGAATTCTGTGGCAG-3′ (SEQ ID NO: 15) and ABL-R1: 5′-CTTTGCCAGGAGCCTAGTGT-3′ (SEQ ID NO: 16)) and the PCR products were subsequently sequenced. The result indicated that highly efficient and precise editing was achieved. The on-target integration of donor sequence was detected in 43% of genomic PCR products of the cells transfected with the ANAGO expression construct based on NgAgo, meanwhile the non-HDR indel incidence was less than 1% (FIG. 2b). The integration of TAAGCTT (stop/Hind III) mutation sequence was further confirmed (FIG. 2c). In order to have a comprehensive assessment of editing events, a 429 bp of PCR amplicon of target site was generated by using a pair of primers (ABL-F2: 5′-TTGGGACCATGTTGGAAGTT-3′ (SEQ ID NO: 17) and ABL-R2: 5′-AGCACTGAGGTTAGAAGCTG-3′ (SEQ ID NO: 18)). The amplicon product was subjected to the Next Generation Sequencing (NGS) (Amplicon-EZ NGS service was provided by GeneWiz, NJ). The total sequence reads were more than 30,000 for each amplicon sample. For the cells transfected with the ANAGO expression construct based on NgAgo, 32.0% of 32,207 sequence reads belong to the precise on-target HDR editing events. In the cells transfected with the other protein expression constructs of the human ANAGO, such as HA-NLS-PfAgo ORF, HA-NLS-TtAgo ORF, and HA-NLS-MjAgo ORF, the percentages of on-target HDR precise editing reads were 23.5%, 19.5% and 10.3% respectively (Table 1). The result also confirmed that the unintended indel events commonly associated with a DNA double-strand break (DSB) were rare, less than 1%.

TABLE 1
Summary of Amplicon
Deep Sequencing Results

| ANAGO | | Precise | | | |
|---|---|---|---|---|---|
| (originated | Target | HDR | | WT | |
| from) | Reads | Reads | HDR (%) | Reads | WT (%) |
| NgAgo | 32207 | 10306 | 32.0 | 5672 | 18 |
| PfAgo | 37355 | 8774 | 23.5 | 10128 | 27 |
| TtAgo | 40280 | 7867 | 19.5 | 14188 | 35 |
| MjAgo | 43696 | 4512 | 10.3 | 19335 | 44 |

Our data indicates the potential clinical application of the AISE technology disclosed herein for the cure of CML and Ph+ALL by permanently inactivation of BCR-ABL oncogene in hematopoietic stem cells isolated from these patients.

### Example 3. Human Histone 2Bc

Histone 2Bc gene is an intronless and house-keeping gene. It encodes a histone 2B subunit. In this example, we want to expand the potential application of the AISE technology. Particularly, we want to see if a short homologous flanking sequence, for example, as short as 50 bps could be used to facilitate the knock-in of a large exogenous DNA fragment (>700 bp) in mammalian cells. We generated a donor fragment H2Bc-mCherry KI by using mCherry ORF fragment as a template and a pair of PCR primers (H2Bc-LH50-mCher: 5′-ACGCAGTGTCCGAAGGTACCAAGGCTGTCACCAAGTATACAAGCTCCAAGATGGTGAGCAAGGGCGAG-3′ (SEQ ID NO: 19); and mCher-H2Bc-RH50: 5′-GTGGCTCTGAAAAGAGCCTTTGAGTTTTAAAGCACCTAAGCACACATTTACTTGTACAGCTCGTCCATGC-3′ (SEQ ID NO: 20)) that incorporated 50 bp of H2Bc sequence as flanking arm on each side of the amplified mCherry fragment. The insertion of donor fragment would result in the in-frame fusion of mCherry open reading frame (ORF) to the last codon of H2Bc (FIG. 3a). The precise integration was detected by genomic PCR reactions with primer pairs H2Bc-F1/mCherry-Rev (H2Bc-F1: 5′-TAACGACATCTTCGAGCGCA-3′ (SEQ ID NO: 21); and mCherry-Rev: 5′-TACGACACTGCATTACGGGG-3′ (SEQ ID NO: 22)) and mCherry-For/H2Bc-R1 (mCherry-For: 5′-CCTTTCCCACAACGAGGACT-3′ (SEQ ID NO: 8); and H2Bc-R1: 5′-TGTGAGACTTGAGTGGCTCTG-3′ (SEQ ID NO: 23)) that amplified the 5′ and 3′ junctional regions respectively. The three human ANAGO constructs (NLS-NgAgo ORF, HA-NLS-PfAgo ORF, and HA-NLS-TtAgo ORF) were all able to facilitate the precise knock-in of mCherry in Histon 2Bc (FIG. 3b). Moreover, the expression of H2Bc-mCherry fusion protein was observed in live cells 72 hours post transfection (FIG. 3c).

### Example 4. Human CD274/PD-L1

High expression of PD-L1, which is also known as CD274, has been associated with tumor cells in advanced stage tumors such as liver cancer (Xu Y, Poggio M, Jin H Y, etc. (2019) Translation control of the immune checkpoint in cancer and its therapeutic targeting. Nature Medicine, 25, 301-311). PD-L1 blockade is a potential form of cancer immunotherapy. It aims to disrupt the activation of the PD-1/PD-L1 axis, which is likely to serve as a mechanism for tumor evasion of host tumor antigen-specific T-cell immunity. In this example, we explored the possibility to inactivate PD-L1 permanently in a tumor cell by AISE approach. A single-stranded oligo CD274-HDR1 72mer donor molecule (5′-GTGAAATTGCAGGATGCAGGGGTGTACCGCAAGCTTGCTAGCGCATGATCAGCTATGGTGGTGCCGACTACA-3′ (SEQ ID NO: 24)) was designed to introduce a premature stop codon as well as Hind III and NheI sites into the exon 3 region of human CD274 gene (FIG. 4a). We co-transfected donor DNA and plasmid DNA of a humanized ANAGO expression construct into HEK293 cells. The genomic DNA of transfected cells was harvested at 96 hours post transfection. The genomic region containing the entire exon 3 was amplified by PCR with a pair of specific primers (CD274-F3: 5′-AGCATTTACTGTCACGGTTCCC-3′ (SEQ ID NO: 25) and CD274-R2: 5′-AAAGATCAGGCCTCTCATCTATAA-3′ (SEQ ID NO: 26)) for amplicon deep-sequencing (GeneWiz, New Jersey). The data analysis revealed that over 18% reads showed the on-target HDR editing in the exon 3 of CD274 gene in HEK293 cells transfected with the human ANAGO construct derived from PfAgo sequence (FIG. 4b).

In order to evaluate the difference between the AISE with both a guide molecule and a donor molecule and the AISE with a donor molecule alone without a guide molecule, a separate comparison experiment under the same condition as above with the exception of the addition of a guide molecule (5′-TGCAGGGGTGTACCGCTGCATGAT-3′ (SEQ ID NO: 27)) that is targeted to the desired mutation site (FIG. 4c) in the presence of the donor molecule described above. In this case, the deep-sequencing data analysis revealed that only 2.7% of the reads showed the desired on-target HDR editing in the exon 3 of CD274 gene in HEK293 cells transfected with the human ANAGO construct derived from PfAgo sequence (FIG. 4c).

Based on the above results, the percentage of the total HDR and/or the precise HDR using AISE in the presence of both a guide molecule and a donor molecule was significantly reduced as compared to that of the AISE using a donor molecule alone without a guide molecule, >18% vs 2.7% respectively. These results clearly demonstrate that not only is a guide molecule not required for ANAGO-induced precise genomic sequence editing (AISE), but also the addition of a guide molecule in the AISE system may lead to significantly lower percentage of desired HDR as compared to AISE system with a donor molecule alone without a guide molecule. These data showed feasibility of using the AISE technology disclosed herein without a guide molecule to enhance natural immune response by inactivating PD-L1 gene in tumor tissues, and apparent advantages without using a guide molecule.

### Examples for Precise Genome Editing in Human Pluripotent Stem Cells

### Materials and Methods

Constructs of ANAGO expression cassette: The expression of fusion open reading frame of ANAGO was driven by EFlalpha promoter in a mammalian expression vector (SynBio Tech, New Jersey). The dominant negative form of human TP53 gene (P53DD) was synthesized and subcloned into pCDNA3.1 mammalian expression vector. Human Rad51 siRNA (Cat #SASI_Hs01_00018925) was ordered from Millipore-Sigma

Cell culture and transfection: iPSC line 400067 cells were cultured under a feeder-free condition with StemFit B04 medium (AMSBIO) supplemented with 40 ng/ml of FGF-2. Cells were seeded into a 12-well plate coated with iMatrix-511 (AMSBIO, Boston, Mass.) one day before transfection. Cells were transfected at about 50% confluence using Lipofectamine Stem (Thermo Scientific). Specifically, the cells were co-transfected with 1000 ng of a human ANAGO-expressing plasmid and 200 nM donor oligos either individually or in combinations. At 72 hours post-transfection, the genomic DNA was isolated and analyzed by PCR and sequencing to detect the gene editing event. Subsequently, the amplicon deep sequencing and data analysis were carried out.

Applications of AISE in hPSCs

As of today, only 5% of human genes have been linked to known biological phenotypes. Vast majority of human genome are still mysterious for their functions. The introduction of desired genetic changes to investigate their phenotypic effects via reverse engineering approach has been proven to be extremely powerful to decipher gene functions in the mouse genome through gene targeting in embryonic stem cells. To implement this approach in hPSCs using AISE technology described herein, different methods could be developed and used, such as the ones described below.

### Gene Knockout Approach

The simplest approach to address gene function is completely inactivating the gene to be studied by introducing a nonsense mutation in its protein coding sequence. This can be done with AISE technology by a donor template harboring a stop codon to be inserted into the gene of interest, as demonstrated in the following examples.

### Example 5. Human BCR-ABL

The Philadelphia (Ph) translocation t(9; 22)(q34; q11) leads ABL1 to fuse with BCR. The resulting BCR-ABL fusion gene is the main reason causing chronic myelogenous leukemia (CML) and Ph+ acute lymphoblastic leukemia (ALL). BCR-ABL fusion gene encodes a constitutively active cellular tyrosine kinase that is necessary and sufficient to induce and maintain leukemic transformation. The age-adjusted incidence is 1.6 per 100,000 population members. Small molecular inhibitors such as Gleevec and Sprycel have been developed and approved for clinical treatments. However, drug resistance often occurs in many patients receiving treatment.

Since the main cause of disease is the hyper kinase activity of the fusion protein, an insertion of a premature stop codon before the coding region of kinase domain should result in a truncated product that lacks the kinase domain and thereby inactivates the BCR-ABL fusion gene. Using AISE technology described herein to disable BCR-ABL fusion gene could be carried out in a patient's bone marrow hematopoietic stem cells (HSCs) ex vivo. This approach may cure the diseases permanently with a single treatment for CML and Ph+ ALL patients.

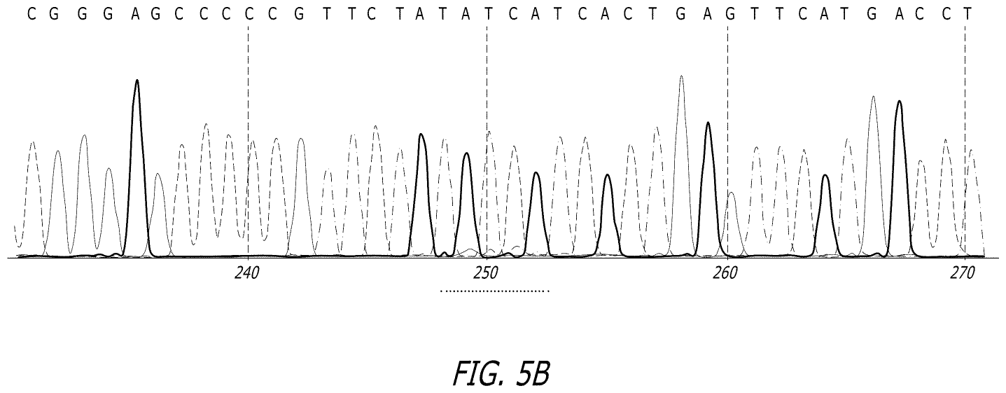

We designed a donor molecule ABLex6stop-H3 derived from human ABL1 gene exon sequence (5′-GGCAGGGGTCTGCACCCGGGAGCCCCCGTTCTAAGCTTTCACTGAGTTCATGACCTACGGGAACCTCCTG-3′ (SEQ ID NO: 6)), a 70mer single-stranded DNA (ssDNA) oligo, which converted the residue Tyr312 codon TAT into a stop codon TAA. The subsequent four nucleotides ATCA were deleted and replaced with GCTT to make up a Hind III site. The engineered sites are flanked by 33 and 32 nucleotides of short homology arms on the left and right sides correspondingly (FIG. 5a). To test whether this single-stranded DNA (ssDNA) oligo harboring 5 altered bases could be used to precisely delete target nucleotides and insert the compound stop codon/Hind III site into ABL1 exon 6 region in cultured human iPSCs, we transiently co-transfected the donor oligo with a human ANAGO construct of HA-NLS-PfAgo ORF described above (with nucleic acid sequence of SEQ ID NO:2-DNA sequence of a human ANAGO of HA-NLS-PfAgo ORF). Moreover, in order to facilitate ANAGO-mediated homology-directed DNA strand exchange, a negative dominant P53 mutant construct P53DD and small interfere RNA (siRNA) against Rad51 gene were also included in the transient transfection cocktail (Wang A T., et al., A dominant Mutation in Human RAD51 Reveals Its Function in DNA Interstrand Crosslink Repair Independent of Homologous Recombination. (2015) Mol Cell 59(3):478-90; Watanabe T. et al., Highly efficient induction of primate iPS cells by combining RNA transfection and chemical compounds. (2019) Genes Cells 7:473-84). The genomic DNA of transfected cells was harvested at 72 hours post transfection. Due to the low efficiency of transfection in human PSCs, relatively small portion of PCR products showed precise sequence alteration by Sanger sequencing (FIG. 5b). In order to have a comprehensive assessment of the editing events, we amplified the target region by PCR with a pair of specific primers (ABL-F2: 5′-TTGGGACCATGTTGGAAGTT-3′ (SEQ ID NO: 17) and ABL-R2: 5′-AGCACTGAGGTTAGAAGCTG-3′ (SEQ ID NO: 18)). The 429 bp amplicon product was subjected to the Next Generation Sequencing (NGS). Over 65,000 reads have been generated. The data analysis indicated that there were 2.9% HDR precise editing events and 0.8% unintended indel events. Since the best efficiency of hPSC transient transfection is usually less than 30%, we estimate that AISE-mediated HDR in the cells received both ANAGO and donor oligo should be happened around 10%. The result also suggests that a potential cure of CML and Ph+ ALL by permanently inactivation of BCR-ABL oncogene via AISE technology is attainable in primary cells.

### Multiplex Gene Editing

In many cases, multiple genetic loci and/or single nucleotide polymorphisms (SNPs) are involved in a biological condition. To model such complex genetic configurations, AISE also offers a significant advantage over other systems for multiplex and fine editing. Since the only variable component is a donor molecule, multiple donor templates can be delivered simultaneously together with ANAGO to a target cell. This approach may facilitate the generation of multiplex genomic editing in hPSCs.

### Example 6

Proprotein convertase subtilisin/kexin type9 (PCSK9), acts in lipoprotein homeostasis. It binds and removes low-density lipoprotein receptors (LDLR). If not binding PCSK9, LDLR will return to the cell surface and can continue to remove LDL-particles from the bloodstream. Agents which block or inhibit the binding between LDLR and PCSK9 can lower LDL particle concentrations. Significantly, heterozygous loss-of-function (LOF) mutations of PCSK9 gene have been found in some individuals. They have a near 88% reduced risk of developing coronary heart disease over a 15-year follow-up period [Cohen J C, Boerwinkle E, et al, 2006, NEJM 354:1264-1272]. Moreover, carriers of LOF mutations such as deltaR97+Y142X, C679X, and R104C+V114A were found to lack PCSK9 expression. These individuals with natural occurring LOF mutations have very low LDL levels in their blood stream without association with any obvious adverse health consequences. This supports the clinical utility of introducing LOF mutations to PCSK9 through gene editing to treat hypercholesterolemia.

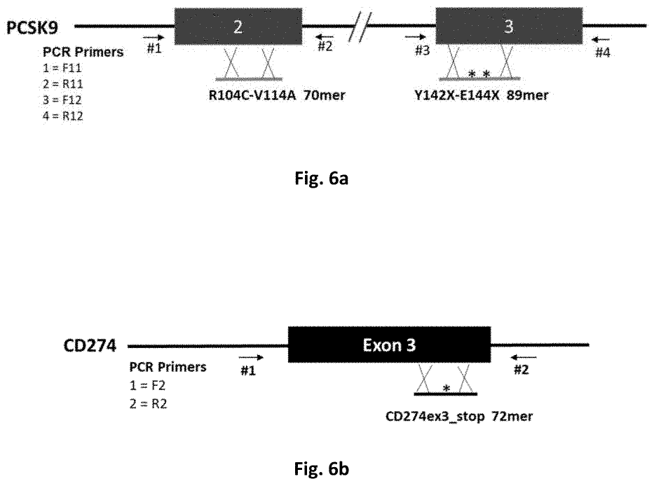

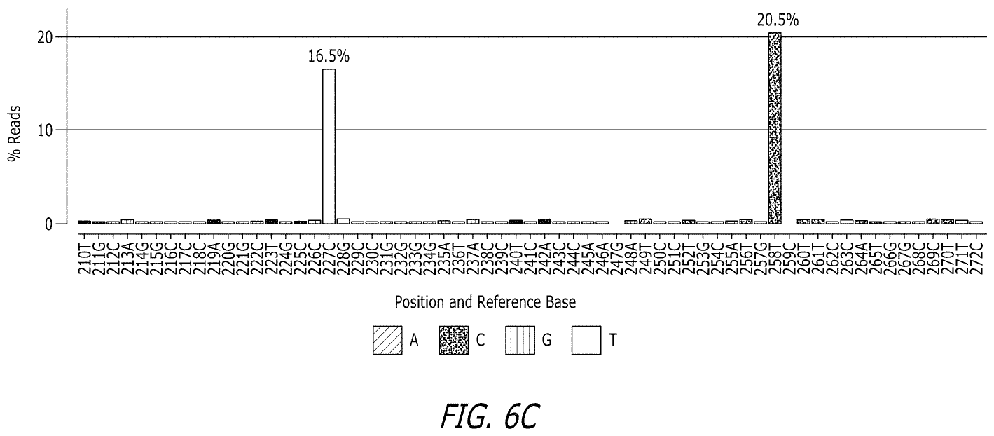

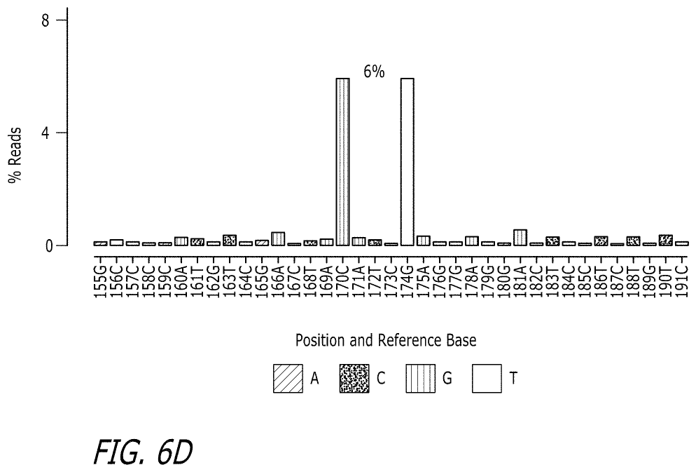

To test if AISE technology can be used to generate the discrete point mutations that mimic PCSK9 natural LOF mutations R104C (CGC>TGC)+V114A (GTC>GCC) in human PSCs, a 70mer ssDNA donor molecule PCSK9ex2_R104C-V114A targeting PCSK9 exon 2 to make the two point mutations (the boxed) (5′-CCTGCAGGCCCAGGCTGCC [symbol] GCCGGGGATACCTCACCAAGATCCTGCATG [symbol] CTTCCATGGCCTTCTTCCT-3′ (SEQ ID NO: 5) and FIG. 6a) was co-transfected with ANAGO expression construct HA-NLS-PfAgo ORF described above (with nucleic acid sequence of SEQ ID NO:2-DNA sequence of a human ANAGO of HA-NLS-PfAgo ORF). The two altered nucleotides are located at the 20th and 40th base positions of the 70mer respectively. The results showed that about 16.5% of sequence reads contained the intended R104C mutation (C/T at the position 20) and 20.5% of reads showed V114A mutation (T/C at the position 40) incorporated into the PCSK9 allele in human PSCs (FIG. 6c). The slightly higher mutation rate of V114A than R104C is likely due to the length of short arm of homologous sequence (30 nts vs 19 nts) in the donor molecule. This result also validates the notion that a short homologous region of 19 bases on each side of modified bases should be sufficient enough to lead to an intended homology directed recombination (HDR).

To test whether AISE technology can be used to simultaneously edit two unrelated genes in the genome of human PSC, we transiently co-transfected ANAGO expression construct along with two different donor molecules targeting two different genes: 1) a 89mer ssDNA donor PCSK9ex3_Y142X-E144X (5′-TCTCTGGCTTCTGCAGGCCTTGAAGTTGCCCCATGTCGAC [symbol] ATC [symbol] GAGGACTCCTCTGTCTTTGCCC AGAGCATCCCGTGGAACC-3′ (SEQ ID NO: 12)) targeting human PCSK9 exon 3 (FIG. 6a) to introduce Y142X and E144X, two point-nonsense mutations (the boxed, TAC to TAG and GAG to TAG respectively); and 2) a 72mer ssDNA donor molecule CD274ex3_stop (5′-GTGAAATTGCAGGATGCAGGGGTGTACCGC [symbol] GCATGATCAGCTATGGTGGTGCCGAC TACA-3′ (SEQ ID NO: 24)) targeting CD274 exon 3 (FIG. 6b) to insert 12 nucleotides (the underlined) containing the stop codon TAG (the boxed). The deep sequencing analysis of amplicon products from the pooled genomic DNA of transfected hPSCs demonstrated that among the sequence reads, about 6% of sequence reads contained the intended Y142X-E144X mutations in PCSK9 exon 3 region (FIG. 6d), and about 0.23% of sequence reads had the precise HDR-mediated insertion of 12 bases including the stop codon in the exon 3 of CD274 gene.

DNA homology recombination is depended on the accessibility of target region of chromatin. The transcription active genes are often associated with an open form of chromatin structure, while the inactive ones with a closed form of chromatin structure. We noticed that the transcription activity of CD274 is very low and its transcripts were hardly detectable in hPSCs. In contrast, PCSK9 transcripts were readily detectable. It suggests that CD274 is likely wrapped in a relatively compact chromatin structure, which in turn could hinder the AISE-mediated HDR and result in the reduced editing efficiency in hPSCs. We believe that if the expression of two unrelated genes in the genome of human PSCs are both easily detectable, much easier than CD274, then the efficiency of the AISE-mediated HDR editing of these genes in hPSCs would be much higher than that of CD274, and/or they may have similar efficiency.

These results demonstrate that multiplex genomic editing can be readily accomplished by AISE technology without using a drug selection for enrichment. Moreover, AISE technology can be used to correct a specific disease-causing point-mutation in patient-derived hPSCs.

### Perspectives of AISE Technology or Approach

Unlike Cas proteins, which only exist in prokaryotes, Argonaute proteins are conserved through evolution and can be identified in virtually all species. In mammalian cells, endogenous Argonaute protein is considered to be a major component of the RNA-induced silencing complex (RISC), which mediates RNA interference. Argonaute uses a micro RNA (^{˜}22 nt) as a guide for identifying complementary target mRNA. Interestingly, an evolutionarily related enzyme in prokaryotes, Argonaute of the bacterium Thermus thermophiles (TtAgo), was found to be able to use DNA-guided DNA interference as a defense mechanism to protect its host against foreign DNA. TtAgo bound with 5′-phosphorylated single-stranded DNA guide (13-25 nucleotides in length), effectively cleaved a foreign complementary DNA in vivo. Previously, Zhao was among the pioneers who had utilized gene swamping experiments to demonstrate that evolutionarily conserved proteins, such as homeodomain-containing proteins, were not only conserved in structures, but also could functionally act in a similar fashion in a species that was diverged from a common ancestor over millions of years ago (Zhao J J, Lazzarini R A, Pick L. The mouse Hox-1.3 gene is functionally equivalent to the Drosophila Sex combs reduced gene. Genes Dev. 1993,7: 343-354).

As shown herein the Argonaute gene sequences of Natronobacterium gregoryi [NgAgo], Pyrococcus furiosus [PfAgo], Thermus thermophilus [TtAgo], and Methanocaldococcus jannaschii [MjAgo] were modified to that of SEQ ID NO:1, SEQ ID NO:2, SEQ ID NO:3, SEQ ID NO:4 correspondingly. These modifications resulted in enhanced expressions and/or activities of these humanizedANAGO in human cells. The human ANAGO described herein acts as a DNA directed gene editing nuclease in human cells.

The gene sequences of other microbial Argonaute proteins, such as Clostridium butyricum [CbAgo], and Limnothrix rosea [LrAgo] may be also engineered in a similar fashion to generate species-specific ANAGO to eukaryotes for gene editing in eukaryotic cells, such as human cells.

The AISE technology described herein shall have at least following distinctive advantages over RNA-guided CRISPR/Cas approach:
1) The homology-directed repair or replacement (HDR) of AISE approach generally lead to measurable to high percentage of HDR editing event with superior precision, speed, and throughput, and without concerning about any off-target effect. It is different from the non-homologous end-joining (NHEJ)-mediated gene editing, which is error-prone and mainly utilized in CRISPR/Cas system.
2) A very versatile donor template, which could either be single-stranded or double-stranded DNA molecule, can be used in the ANAGO induced gene editing. The donor nucleic acid can accommodate as minimal as a single base substitution or as large as at least 1500 nucleotides of exogenous sequence. In fact, it has been reported recently that a single-stranded oligodeoxynucleotides (ssODNs)-mediated knock-in of a donor fragment in mammalian cells occurs via HDR and is more efficient than using a double-stranded donor fragment.
3) ANAGO proteins are much smaller than Cas nucleases. It could be easily fit into an adeno-associated viral (AAV) vector, a very promising in vivo delivery system for clinical applications.

Unlike the CRISPR/Cas system, the precise genomic sequence editing using the AISE technology described herein normally results in very low numbers of imprecise editing events, since the AISE technology is mainly depended on homology directed recombination. The imprecise editing associated with AISE can be as low as about 0%, about 0.5%, about 0.8%, about 1.3%, about 0%-4%, about 0%-0.1%, about 0.1%-4%, about 0.1%-3%, about 0.1%-2%, about 0.1%-1%, about 0%-1%, about 0%-2%, about 0%-3%, about 0%-4%, about 0.1%-0.5%, about 0.1%-0.2%, about 0.2%-0.3%, about 0.3%-0.4%, about 0.4%-0.5%, about 0.5%-1%, about 0.5%-0.7%, about 0.7%-1%, about 1%-1.5%, about 1.5%-1.7%, about 1.7%-2%, about 1.5%-2%, about 2%-2.5%, about 2.5%-3%, about 3%-3.5%, or about 3.5%-4%, of the genomic PCR products, or any percentage in a range bounded by any of the above values. The AISE technology with very low or lack of imprecise editing provides a very safe, powerful and desirable tool for precise genomic sequence editing, which enables wide applications using AISE to treat many gene associated diseases, disorders, or conditions, both known or yet to be known.

Furthermore, the AISE technology described herein has very low rate of the unintended indel events including both unintended deletion and unintended insertion at or around the target region, such as about 0%-2%, about 0%-0.1%, about 0.1%-2%, about 0%-1%, about 0.1%-1%, about 1%-2%, about 1%-1.5%, about 1.5%-2%, about 0%-0.1%, about 0%-0.5%, about 0.5-1%, about 0.1%, about 0.2%, about 0.3%, about 0.4%, about 0.5%, about 0.6%, about 0.7%, about 0.8%, about 0.9%, or about 1%, or any percentage in a range bounded by any of the above values.

The rate of the unintended sequence insertion event at or around the target region associated with AISE is very low, such as about 0%-2%, about 0%-0.1%, about 0.1%-2%, about 0%-1%, about 0.1%-1%, about 1%-2%, about 1%-1.5%, about 1.5%-2%, about 0.1%-2%, about 0.1%-1%, about 0.1%-0.5%, about 0.5%-1%, about 0.1%-0.3%, about 0.3%-0.5%, about 0.5%-0.7%, about 0.7%-0.9%, about 0.9%-1%, about 0.2%-0.4%, about 0.4%-0.6%, about 0.6-0.8%, about 0.8%-1.0%, about 0.1%, about 0.2%, about 0.25%, about 0.3%, about 0.4%, about 0.5%. about 0.55%, about 0.6%, about 0.7%, about 0.8%, about 0.9%, or about 1%, or any percentage in a range bounded by any of the above values.

The rate of the unintended sequence deletion event at or around the target region associated with AISE is very low, such as about 0%-2%, about 0%-0.1%, about 0.1%-2%, about 0%-1%, about 0.1%-1%, about 1%-2%, about 1%-1.5%, about 1.5%-2%, about 0.1%-2%, about 0.1%-1%, about 0.1%-0.5%, about 0.5%-1%, about 0.1%-0.3%, about 0.3%-0.5%, about 0.5%-0.7%, about 0.7%-0.9%, about 0.9%-1%, about 0.2%-0.4%, about 0.4%-0.6%, about 0.6-0.8%, about 0.8%-1.0%, about 0.1%, about 0.2%, about 0.25%, about 0.3%, about 0.4%, about 0.5%. about 0.55%, about 0.6%, about 0.7%, about 0.8%, about 0.9%, or about 1%, or any percentage in a range bounded by any of the above values.

The very low rate or none of unintended indel events associated with the AISE technology provides a very significant benefit in eliminating or significantly reducing the risks associated with the unintended indel events in gene therapy. Thus, AISE technology described herein could benefit a huge population of patients who have gene associated diseases, disorders, or conditions, both known or yet to be known.

The rate of the homologous directed replacement (HDR %) of the on-target integration of donor sequence detected with various methods using different human ANAGO are varied for different targets. The HDR % can be about 1%-100%, about 1%-95%, about 1%-90%, about 1%-80%, about 1%-70%, about 1%-60%, about 1-50%, about 1-5%, about 5-10%, about 10-15%, about 15-20%, about 20-30%, about 30-40%, about 40-50%, about 50-60%, about 2-4%, about 4-6%, about 6-8%, about 8-10%, about 10-20%, about 10-30%, about 30-50%, about 1%, about 3%, about 4%, about 4.2%, about 7.1%, about 10.3%, about 18%, about 19.5%, about 23.5%, about 32%, about 43%, at least 3%, at least 4%, at least 5%, at least 7%, at least 10%, at least 15%, at least 18%, at least 20%, at least 30%, or at least 40%, or any HDR % in a range bounded by any of the above values for the genomic PCR products of the cells transfected with various human ANAGO expression constructs. The fact that some embodiment using the AISE described herein has demonstrated 43% HDR of precise sequence editing indicating tremendous potential of AISE in gene therapy. The ability to manipulate any genomic sequence by AISE technology described herein may have far-reaching implications and opportunities in developing new treatments for many different human diseases and disorders. Some major applications contemplated herein are:

### Cellular Immunotherapy

Cancer immunotherapy involves or uses components of the immune system such as antibodies and T cells to treat cancer patients. Recently, impressive treatment results have been reported in some cases of lymphoma, leukemia and melanoma with adoptive T-cell immunotherapy, in which autologous T cells are engineered to attack cancer antigen ex vivo and transferred back to the patient. The T-cell immunotherapy could be further enhanced via expressing synthetic receptors known as chimeric antigen receptors, or CARs, and knocking out the endogenous T-cell receptors with engineered nucleases such as the human ANAGO described herein. In addition, knocking out the human leukocyte antigen (HLA) via gene editing could avoid immune rejection of allogeneic cell therapy and offer a universal off-the-shelf cell therapy product.

Another useful application is to increase T-cell effector function and broadly enabling immunotherapy for diverse cancer types by knocking out key genes of checkpoint inhibitor pathways such as CD274/PD-L1 (see the example 4 above) and CTLA-4.

### Germline and Embryonic Engineering

Cystic fibrosis (CF) is a hereditary disease that affects the lungs and digestive system. The body produces thick and sticky mucus that can clog the lungs and obstruct the pancreas. Cystic fibrosis can be life-threatening, and people with the condition tend to have a shorter-than-normal life span. Cystic Fibrosis is caused by any one or combination of numerous mutations affecting the function of the Cystic Fibrosis Transmembrane Conductance Regulator (CFTR). Currently, there is no cure for the disease, but the symptoms can be mitigated through therapy. Mutations can affect different parts of the pathway thereby leading to distinct natural history and calling for unique therapies. In all cases, gene-editing to correct the mutation before a child is born would greatly increase the quality of life of the future child. Under the responsible ethical guidance, the AISE technology would be a method of choice for gene-editing in these cases because it can work easily in germ cells or fertilized eggs, has minimal off-target effects, and can easily correct simple mutations via homologous recombination. For example, if a set of germ cells or fertilized eggs are sequenced in vitro and found to have the F508del mutation, the CFTR misfolding that would be expected to occur and affect a person's health can be reverted to normal form by using ANAGO mediated precise gene-editing technology.

### Optogenetics

Optogenetics is a research tool that is commonly found in neuroscience laboratories. In this context, the ability of Argonaute to swap out large loci such as mCherry (700 base pairs) can be fully taken advantage of. Mice in optogenetics experiments are bred to have a genetic cassette containing ion channels possessing a light sensitive property. Rhodopsins are generally used as the light sensitive segment. Viral vehicles are typically used to deliver the gene engineering tool to introduce the channel Rhodopsin, flanked by a promoter, to the cell types of interest. In this case, ANAGO in a form of RNA could be placed in the viral capsid and injected into the portion of the mouse brain that is under study. The genetic engineering can also be done upstream in the germline cells of an animal model, and application for which AISE technology would equally be well-suited. A probe with the capability of delivering targeted beams of light is then inserted into the animal brain (a probe containing an optic fiber, commonly). Once the rig is cemented in place, researchers can toggle the light between on and off and change the activity of the neurons in different settings, environments, and in the presence of novel stimuli. Thus, ANAGO mediated gene editing technology has the ability of accelerating discoveries in the realm of neuroscience.

### Antiviral Therapy

Gene editing strategy has been used to remove the integrated viral DNA sequence from a host genome or knock out CCR5, a coreceptor used for primary HIV infection. Currently, several ongoing clinical trials are evaluating this approach in HIV-positive patients. Early study results provide promising proof-of-principle of a gene-editing approach in humans, which show safe engraftment and survival of CCR5-modified T cells and control of viral load in some patients. The ANAGO mediated gene-editing platform could also be applied to attack viral genomes of various DNA viruses such as HIV, hepatitis B virus, herpes simplex virus, and human papilloma virus etc. To circumvent the high mutability of viral targets, several donor molecules could be used simultaneously to target multiple critical sites in the viral genome.

### Liver-Targeted Gene Editing

Liver is probably one of the most accessible organs for applying gene-editing technology to treat many different diseases. At one hand, the AISE technology could be used to correct mutations caused severe diseases including clotting disorders such hemophilia A and hemophilia B, as well as lysosomal storage disorders such as Fabry disease, Gaucher disease, Pompe disease, von Gierke disease, and Hurler and Hunter syndromes. On the other hand, the disruption of particular genes in the tissue may also have a health beneficial effect. For example, reducing PCSK9 activity is correlated with decreased LDL level. In contrast to continuous administration of PCSK9 inhibitors, a liver specific targeted PCSK9 knockout or variant substitutions could lead to long-lasting effect of lowering cholesterol levels.

### Blindness Treatment

Another highly accessible organ for applying gene-editing technology is eye. Recent successes in clinical trials for the treatment of Leber Congenital Amaurosis type 2 (LCA2) have raised hope of using gene therapy to treat blindness caused by genetic mutations. LCA is the leading cause of childhood blindness and is caused by mutations in at least 18 different genes. Other autosomal dominant disorders such as forms of retinoblastoma, primary open angle glaucoma, retinitis pigmentosa and Fuchs endothelial corneal dystrophy, could potentially be treated by targeted editing of mutation sites. More importantly, the proven safety of adeno-associated virus (AAV) delivery in eyes and the compact size of the ANAGO system make it a particularly attractive gene-editing strategy in clinical settings.

### Introduction of Large Genetic Modifications

AISE technology can also be used to insert a large DNA fragment, such as a fluorescent reporter, drug selection marker, recombinase, or protein tag, into a target locus. The insertion of a large foreign DNA fragment into a safe harbor site of human genome such as AAVS1 locus has been successfully demonstrated by using some existing gene editing technologies such as ZFN, TALEN and CRISPR-Cas systems. In addition, the creation of gene specific knock-in reporter lines is particularly useful for real-time monitoring gene expression, cell-lineage tracing, differentiation screens, drug screening, and cell sorting of a targeted population for further molecular characterization. For example, a donor-targeting fragment carrying the homology arms, the reporter gene, and a selection marker allows enriching targeted events through drug selection, may be introduced in the presence of ANAGO.

### Conditional Gene Knockouts

The ability to inactivate a target gene in a temporal or tissue-specific manner may be important for studying human genes with pleiotropic effects or required for the differentiation during development. Human three-dimensional (3D) organoid derived from hPSCs in vitro cultures have recently come to the forefront as superior disease modeling mechanisms than flat monolayer two-dimensional (2D) cultures. Organoids are capable of self-organizing into primary tissue-like in constitution. Organoid cultures are in some cases a better model for understanding cell type specificity as well as cell-to-cell interactions in a temporal or tissue-specific manner than flat cultures alone. It allows researchers to study the developmental process of a certain tissue from beginning to end. Thus, AISE technology could be used to establish a causal relationship between a genetic mutation and stem cell behavior and to discover essential pathways in stem cell differentiation. Another application of AISE technology is in some instances to allow for fluorescent screening of developmental markers within a stem cell population. The AISE technology may be used to create a CRX-GFP reporter iPSC line to differentiate and simultaneously purify photoreceptor progenitor cells.

### Genome Editing of Stem Cells

Stem cells have huge potential in cell-based therapies and regenerative medicine. As differentiated cells are not capable of dividing without limit, introducing stem cells in damaged tissues could help repair them easily. This has great implications in organ transplants, for instance, which is currently limited by the availability of healthy organ donors. The ability to use patient's own stem cells could potentially lower incidences of transplant rejection by the immune system. However, before being transplanted back to a patient, the disease-causing mutations need to be eliminated from the cells. Thus, a reliable and efficient gene editing technology such as AISE technology disclosed herein may greatly facilitate the development of personalized regenerative medicine. For examples, to correct disease-causing mutations in autologous iPSCs for patient-specific treatments. In addition, to generate a universally compatible cell source for allogeneic cell therapies without a need of any immunosuppression, AISE technology can be used to create hypoimmunogenic pluripotent stem cells.

Stem cell models of disease provide an unprecedented level of accuracy in disease modeling, as they are derived from human donors rather than model animal species. On the other hand, AISE technology as demonstrated herein can create isogenic cell lines which are a precise control for a genetic disease model of interest. Thus, the use of AISE technology in stem cells as exemplified herein offers tremendous potential and advances a step closer to a reality in treating or cure genetic related diseases or disorders.

AISE technology may be employed as an ex vivo gene editing tool in hematopoietic stem cells (HSCs). HSC therapy was the earliest stem cell therapy to be used in the clinic, replacing defective bone marrow stem cells via harvest from either a donor or umbilical cord blood. For instance, CCR5 is known to be a co-receptor for HIV cellular entry. One can employ AISE technology to disrupt the CCR5 gene in hematopoietic stem cells from HIV positive patients. The patient's immune system could be reconstituted by infusing with these HIV-resistant HSCs.

Broadly speaking, with the advent of the ANAGO mediated precise gene editing technology, manipulating the genome of plants, animals, and particularly human pluripotent stem cells will become an attainable task, allowing to modify a genome with superior precision, speed, and throughput. AISE technology described herein will revolutionize our scientific communities worldwide for both developing experimental models to understand biological processes and strategies for improving wellness of living organisms.

In certain aspects, the de novo chemically synthesized single stranded oligodeoxynucleotide-mediated sequence replacement protocol described herein can be applied to any target site without addition of a guide molecule, thus simplifying genome engineering in living organisms.

In some embodiments, the nucleic acids, compositions and methods described herein are useful for additional applications, non-limiting examples of which include single and multiplex gene knockouts, conditional gene knockouts, generation of knock-in alleles, introduction of small as well as large genetic modifications, generation of large deletions and chromosome engineering, genome-wide screens, transcriptional regulation, genetic modifications of mitochondrial DNA and target mitochondrial diseases, and the like.

Unless otherwise indicated, all numbers expressing quantities of ingredients, properties such as percentages, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

What is claimed is:

1. A synthetic nucleic acid comprising:
a first nucleic acid sequence comprising about 1000 or more contiguous nucleotides,
wherein the first nucleic acid sequence encodes a codon-Adapted Nuclear Argonaute protein (ANAGO) that is a polypeptide, capable of editing a target nucleic acid sequence within a human cell in the presence of a donor nucleic acid without a guide nucleic acid, wherein the ANAGO is species-specific to the human, wherein the ANAGO is attached to a coding sequence of a nuclear localization signal (NLS) peptide;
wherein the first nucleic acid sequence is produced by modifying a second nucleic acid sequence of a microbial species, and wherein the second nucleic acid sequence comprises a coding region that is capable of encoding a microbial Argonaute protein that has endonuclease activities in a microorganism; and
wherein the modifying comprises replacing microbial preferred codons of the second nucleic acid sequence with codons that have preferential usage in the human cell.

2. The synthetic nucleic acid of claim 1, wherein the first nucleic acid sequence comprises at least 85% identity to the nucleic acid sequence of SEQ ID NO:1; SEQ ID NO:2; SEQ ID NO:3, or SEQ ID NO:4.

3. The synthetic nucleic acid of claim 1, wherein the human cell is a human stem cell.

4. The synthetic nucleic acid of claim 1, wherein the first nucleic acid sequence and the second nucleic acid sequence shares less than 85% sequence identity.

5. The synthetic nucleic acid of claim 1, further comprising a promoter operably linked to the first nucleic acid sequence.

6. A composition comprising the synthetic nucleic acid of claim 1, and a donor nucleic acid.

7. The composition of claim 6, wherein the donor nucleic acid comprising:
(i) a desired nucleic acid sequence to be introduced into a target nucleic acid sequence by the ANAGO;
(ii) a 5′-flanking sequence; and
(iii) a 3′-flanking sequence,
wherein the 5′-flanking sequence and the 3′-flanking sequence independently comprise at least 10 consecutive nucleotides that are at least 90% identical to the target sequence located in the genome of a human cell.

8. The composition of claim 7, wherein the human cell is a human stem cell.

9. The composition of claim 7, wherein the 5′-flanking sequence and the 3′-flanking sequence each contain 10 nucleotides to 500 nucleotides.

10. The composition of claim 7, wherein each of the 5′-flanking sequence and the 3′-flanking sequence comprise at least 10 nucleotides that are identical to the target sequence.

11. The composition of claim 6, further comprising one or more of a pharmaceutical acceptable excipient, diluent, additive, or carrier.

12. A method of editing a genome of a human cell comprising:
introducing into the human cell
(i) an ANAGO encoded by the first nucleic acid sequence of claim 1; and
(ii) a donor nucleic acid comprising:
a desired nucleic acid sequence to be introduced into a target nucleic acid sequence, by the ANAGO,
a 5′-flanking sequence, and
a 3′-flanking sequence,
wherein the 5′-flanking sequence and the 3′-flanking sequence are located on opposite sides of the desired nucleic acid sequence and independently comprise at least 10 consecutive nucleotides that are at least 90% identical to the target sequence located in the genome of the human cell.

13. The method of claim 12, wherein the human cell is a human stem cell.

14. The method of claim 12, wherein the first synthetic nucleic acid sequence comprises at least 85% identity to the nucleic acid sequence of SEQ ID NO:1, SEQ ID NO:2, SEQ ID NO:3, or SEQ ID NO:4.

15. The method of claim 12, wherein the donor nucleic acid is a single-strand molecule or a double-strand molecule.

16. The method of claim 12, wherein the 5′-flanking sequence and the 3′-flanking sequence each contain 10 nucleotides to 500 nucleotides.

17. The method of claim 12, wherein each of the 5′-flanking sequence and the 3′-flanking sequence comprise at least 10 nucleotides that are identical to the target sequence.

18. The method of claim 12, wherein the donor nucleic acid comprises a single nucleotide change as compared to the target sequence.

19. The method of claim 12, wherein the genome editing in the human cell is for treating chronic myelogenous leukemia; lowering LDL levels in blood stream; or enhancing the effectiveness of immune therapy against tumor cells in a human being.

20. The method of claim 13, wherein genome editing in the human stem cell is for treating chronic myelogenous leukemia; treating cystic fibrosis; lowering LDL levels in blood stream; or for the use in hematopoietic stem cells (HSCs) therapy to replace defective bone marrow stem cells.

21. The method of claim 13, further comprising introducing into the human stem cell (iii) a dominant negative form of human TP53 gene (P53DD) encoded the synthetic nucleic acid sequence (5′-GGATCCATGCCCCCAGGGAGCACTAAGCGAGCACTGCCCAACAACACCAGCTCCTCTCCCCAGCCAAAG AAGAAACCACTGGATGGAGAATATTTCACCCTTCAGATCCGTGGGCGTGAGCGCTTCGAGATGTTCCGA GAGCTGAATGAGGCCTTGGAACTCAAGGATGCCCAGGCTGGGAAGGAGCCAGGGGGGAGCAGGGCTC ACTCCAGCCACCTGAAGTCCAAAAAGGGTCAGTCTACCTCCCGCCATAAAAAACTCATGTTCAAGACAGA AGGGCCTGACTCAGACAAGCTT-3′ (SEQ ID NO: 45)) that is expressed by a mammalian expression vector.

22. The method of claim 13, further comprising introducing into the human stem cell iV) a small interference RNA molecule target Human Rad51.

23. The method of claim 21, wherein the method increases the gene editing efficiency of the target sequence.

24. The method of claim 22, wherein the method increases the gene editing efficiency of the target sequence.


SEQUENCE LISTING (46 sequences)

SEQ ID NO: 1 - DNA, 2688 bp, Artificial Sequence
  Description of Artificial Sequence Synthetic polynucleotide
atggtgccaaaaaagaagagaaaggtagccaccgtgatcgacctggactccaccaccaccgccgacgagctgacctccggccacacctacgacatctccgtgaccctgaccggcgtgtacgacaacaccgacgagcagcacccccggatgtccctggccttcgagcaggacaacggcgagcggcggtacatcaccctgtggaagaacaccacccccaaggacgtgttcacctacgactacgccaccggctccacctacatcttcaccaacatcgactacgaggtgaaggacggctacgagaacctgaccgccacctaccagaccaccgtggagaacgccaccgcccaggaggtgggcaccaccgacgaggacgagaccttcgccggcggcgagcccctggaccaccacctggacgacgccctgaacgagacccccgacgacgccgagaccgagtccgactccggccacgtgatgacctccttcgcctcccgggaccagctgcccgagtggaccctgcacacctacaccctgaccgccaccgacggcgccaagaccgacaccgagtacgcccggcggaccctggcctacaccgtgcggcaggagctgtacaccgaccacgacgccgcccccgtggccaccgacggcctgatgctgctgacccccgagcccctgggcgagacccccctggacctggactgcggcgtgcgggtggaggccgacgagacccggaccctggactacaccaccgccaaggaccggctgctggcccgggagctggtggaggagggcctgaagcggtccctgtgggacgactacctggtgcggggcatcgacgaggtgctgtccaaggagcccgtgctgacctgcgacgagttcgacctgcacgagcggtacgacctgtccgtggaggtgggccactccggccgggcctacctgcacatcaacttccggcaccggttcgtgcccaagctgaccctggccgacatcgacgacgacaacatctaccccggcctgcgggtgaagaccacctaccggccccggcggggccacatcgtgtggggcctgcgggacgagtgcgccaccgactccctgaacaccctgggcaaccagtccgtggtggcctaccaccggaacaaccagacccccatcaacaccgacctgctggacgccatcgaggccgccgaccggcgggtggtggagacccggcggcagggccacggcgacgacgccgtgtccttcccccaggagctgctggccgtggagcccaacacccaccagatcaagcagttcgcctccgacggcttccaccagcaggcccggtccaagacccggctgtccgcctcccggtgctccgagaaggcccaggccttcgccgagcggctggaccccgtgcggctgaacggctccaccgtggagttctcctccgagttcttcaccggcaacaacgagcagcagctgcggctgctgtacgagaacggcgagtccgtgctgaccttccgggacggcgcccggggcgcccaccccgacgagaccttctccaagggcatcgtgaacccccccgagtccttcgaggtggccgtggtgctgcccgagcagcaggccgacacctgcaaggcccagtgggacaccatggccgacctgctgaaccaggccggcgccccccccacccggtccgagaccgtgcagtacgacgccttctcctcccccgagtccatctccctgaacgtggccggcgccatcgacccctccgaggtggacgccgccttcgtggtgctgccccccgaccaggagggcttcgccgacctggcctcccccaccgagacctacgacgagctgaagaaggccctggccaacatgggcatctactcccagatggcctacttcgaccggttccgggacgccaagatcttctacacccggaacgtggccctgggcctgctggccgccgccggcggcgtggccttcaccaccgagcacgccatgcccggcgacgccgacatgttcatcggcatcgacgtgtcccggtcctaccccgaggacggcgcctccggccagatcaacatcgccgccaccgccaccgccgtgtacaaggacggcaccatcctgggccactcctccacccggccccagctgggcgagaagctgcagtccaccgacgtgcgggacatcatgaagaacgccatcctgggctaccagcaggtgaccggcgagtcccccacccacatcgtgatccaccgggacggcttcatgaacgaggacctggaccccgccaccgagttcctgaacgagcagggcgtggagtacgacatcgtggagatccggaagcagccccagacccggctgctggccgtgtccgacgtgcagtacgacacccccgtgaagtccatcgccgccatcaaccagaacgagccccgggccaccgtggccaccttcggcgcccccgagtacctggccacccgggacggcggcggcctgccccggcccatccagatcgagcgggtggccggcgagaccgacatcgagaccctgacccggcaggtgtacctgctgtcccagtcccacatccaggtgcacaactccaccgcccggctgcccatcaccaccgcctacgccgaccaggcctccacccacgccaccaagggctacctggtgcagaccggcgccttcgagtccaacgtgggcttcctg

SEQ ID NO: 2 - DNA, 2364 bp, Artificial Sequence
  Description of Artificial Sequence Synthetic polynucleotide
atggtgtacccctacgacgtgcccgactacgccccaaaaaagaagagaaaggtagccaaggccaaggtggtgatcaacctggtgaagatcaacaagaagatcatccccgacaagatctacgtgtacagactgttcaacgaccccgaggaggagctgcagaaggagggctacagcatctacagactggcctacgagaacgtgggcatcgtgatcgaccccgagaacctgatcatcgccaccaccaaggagctggagtacgagggcgagttcatccccgagggcgagatcagcttcagcgagctgagaaacgactaccagagcaagctggtgctgagactgctgaaggagaacggcatcggcgagtacgagctgagcaagctgctgagaaagttcagaaagcccaagaccttcggcgactacaaggtgatccccagcgtggagatgagcgtgatcaagcacgacgaggacttctacctggtgatccacatcatccaccagatccagagcatgaagaccctgtgggagctggtgaacaaggaccccaaggagctggaggagttcctgatgacccacaaggagaacctgatgctgaaggacatcgccagccccctgaagaccgtgtacaagccctgcttcgaggagtacaccaagaagcccaagctggaccacaaccaggagatcgtgaagtactggtacaactaccacatcgagagatactggaacacccccgaggccaagctggagttctacagaaagttcggccaggtggacctgaagcagcccgccatcctggccaagttcgccagcaagatcaagaagaacaagaactacaagatctacctgctgccccagctggtggtgcccacctacaacgccgagcagctggagagcgacgtggccaaggagatcctggagtacaccaagctgatgcccgaggagagaaaggagctgctggagaacatcctggccgaggtggacagcgacatcatcgacaagagcctgagcgagatcgaggtggagaagatcgcccaggagctggagaacaagatcagagtgagagacgacaagggcaacagcgtgcccatcagccagctgaacgtgcagaagagccagctgctgctgtggaccaactacagcagaaagtaccccgtgatcctgccctacgaggtgcccgagaagttcagaaagatcagagagatccccatgttcatcatcctggacagcggcctgctggccgacatccagaacttcgccaccaacgagttcagagagctggtgaagagcatgtactacagcctggccaagaagtacaacagcctggccaagaaggccagaagcaccaacgagatcggcctgcccttcctggacttcagaggcaaggagaaggtgatcaccgaggacctgaacagcgacaagggcatcatcgaggtggtggagcaggtgagcagcttcatgaagggcaaggagctgggcctggccttcatcgccgccagaaacaagctgagcagcgagaagttcgaggagatcaagagaagactgttcaacctgaacgtgatcagccaggtggtgaacgaggacaccctgaagaacaagagagacaagtacgacagaaacagactggacctgttcgtgagacacaacctgctgttccaggtgctgagcaagctgggcgtgaagtactacgtgctggactacagattcaactacgactacatcatcggcatcgacgtggcccccatgaagagaagcgagggctacatcggcggcagcgccgtgatgttcgacagccagggctacatcagaaagatcgtgcccatcaagatcggcgagcagagaggcgagagcgtggacatgaacgagttcttcaaggagatggtggacaagttcaaggagttcaacatcaagctggacaacaagaagatcctgctgctgagagacggcagaatcaccaacaacgaggaggagggcctgaagtacatcagcgagatgttcgacatcgaggtggtgaccatggacgtgatcaagaaccaccccgtgagagccttcgccaacatgaagatgtacttcaacctgggcggcgccatctacctgatcccccacaagctgaagcaggccaagggcacccccatccccatcaagctggccaagaagagaatcatcaagaacggcaaggtggagaagcagagcatcaccagacaggacgtgctggacatcttcatcctgaccagactgaactacggcagcatcagcgccgacatgagactgcccgcccccgtgcactacgcccacaagttcgccaacgccatcagaaacgagtggaagatcaaggaggagttcctggccgagggcttcctgtacttcgtg

SEQ ID NO: 3 - DNA, 2109 bp, Artificial Sequence
  Description of Artificial Sequence Synthetic polynucleotide
atggtctacccctacgacgtgccagattacgcccctaagaaaaagcggaaggtggccaaccacctgggcaagacagaggtgttcctgaacagattcgccctgcggcctctgaaccctgaggaactcagaccttggcggctggaagtggtgctggatcctccacctggacgcgaggaagtgtatcctctgctggctcaagtggctcggagagctggcggagtgacagttagaatgggagatggcctggccagctggtccccacctgaagttcttgtgctggaaggcaccctggccagaatgggccagacatacgcctaccggctgtaccccaaaggcagaaggcctctggatcccaaggatcccggcgagagatctgtgctgtctgccctggctagacggctgctgcaagagagactgagaaggctcgaaggcgtgtgggtggaaggactggccgtgtacagaagagagcacgccagaggacctggctggcgagttcttggcggagctgttctggatctgtgggtgtcagatagcggcgcctttctgctggaagtcgaccccgcctatagaatcctgtgcgagatgagcctggaagcttggctggctcagggacaccctctgcctaaaagagtgcggaacgcctacgacagacggacctgggaactgctgagactgggcgaagaggaccccaaagaacttcctctgcctggcggactgagcctgctggattaccacgcctctaagggcagactgcagggcagagaaggtggaagagtggcctgggttgccgatcctaaggaccccagaaagcccattcctcacctgacaggactgctggtgcctgtgctgaccctggaagatctgcacgaggaagagggatctctggccctgtctctgccttgggaagagagaagaaggcggaccagagagatcgccagctggatcggaagaaggcttggcctgggaacacctgaggctgttagagcccaggcctacagactgagcatccccaagctgatgggccgcagagccgtgtctaaacctgccgatgctctgagagtgggcttctacagagcccaagagacagccctggctctgctcagacttgatggcgctcaaggctggcccgagtttctgagaagggctctgctgagagcctttggagcctctggcgcttctctgagactgcacacactgcacgcccatccttctcagggcctcgcctttagagaggctctgagaaaggccaaagaagagggcgttcaggccgtgctggttctgacacctcctatggcatgggaagatcggaaccggctgaaagccctgctgctcagagagggactgcctagccagatcctgaacgtgcccctgagagaagaggaacggcacagatgggagaatgccctgctgggcctgctggccaaagctggacttcaagtggttgccctgtccggcgcctatcctgctgaactggctgtgggatttgacgctggcggcagagagagcttcagatttggaggcgctgcttgtgccgttggcggagatggtggacatctgctgtggacactgcctgaagctcaggccggcgaaagaatccctcaagaggtcgtgtgggacctgctcgaagaaaccctgtgggccttcagaagaaaggccggcaggctgccttcaagagtgctgctcctgagagatggcagagtgccccaggatgagtttgccctggcactggaagccctggcaagagagggaattgcctacgacctggtgtccgtgcggaaatctggtggcggaagagtgtaccccgtgcaaggcagactggccgatggactgtatgtgcctctggaagataagaccttcctgctgctgaccgtgcaccgggactttagaggcacacccagacctctgaagctggtgcatgaagccggcgacacacctctcgaagctctggcccaccagatctttcacctgaccagactgtaccccgccagcggctttgcctttcctagactgcctgctcctctgcacctggccgacagactggtcaaagaagtgggccgcctgggcatcagacacctgaaagaggtggaccgcgagaagctgttcttcgtg

SEQ ID NO: 4 - DNA, 2202 bp, Artificial Sequence
  Description of Artificial Sequence Synthetic polynucleotide
atggtctacccctacgacgtgccagattacgcccctaagaaaaagcggaaggtggcctttaccatggtgctgaacaaagtgacctacaagatcaacgcctataagatcaaagaggaattcatccccaaagaggtgcacttctaccggatcaagagcttcgtgaacgaggccttcaacttctacagattcgtgaacttctacggcggcatgatcatcaacaagaaagacaagtccttcgtgctgccctacaaggtggacaacaaggtgctgaagtacaaggacggcaacaacgagatccccatcgacatcgagtacatcaagagcctgaagctcgagtacgtgaagcccgagatcgccgagaagcttgtgcggggctatctgaagtccgtgcacaagatcgagcccgagctgagccggatcatcaagaacatccggaagcacaaggtggtggaaaacatcaaggtggaaagctactgcgagtacgaagtgaagaagcacgacggcgactactacctgatcctgaacttcagacacaccgccagcatcaccaagcacctgtgggacttcgtgaatagagacaaggccctgctggaagagtacgtgggcaagaagatcatcttcaagcccaatcctaaagtgcggtacaccatcagcctggtggacgccccaaatcctcagaaaatcgaggaaatcatgagccacatcatcaagtactacaagtggagcgaggacatggtcaagagcaccttcggcgagatcgactacaaccagcctatcatgtactgcgaggaaattctggaacccttcgcaccccagttctgcaacctggtgttctacatggacgagctggacagctacatcctgaaagagctgcagagctactggcggctgagcaacgagaacaagggcaagatcattaacgagattgccaagaaactgcggttcatcgacaacacgcccaaagaactggaattcatgaagttcaacaacaccccgctgctggtcaaggacgtgaacaagaaccccaccaagatctacagcaccaacacactgttcacctggatctacaatcagaacgccaaaatctacctgccttacgacgtccccgagatcatccggaacaagaatctgctgacctacatcctcatcgacgaagagatcaaggatgagctgaaggccatcaaggacaaagtcaacaagatgttccgcaactacaacaagatcgccaacaagaccgagctgcccaagttcaactacgccaaccggtggaagtactttagcaccgacgacatccggggcatcatcaaagagattaagagcgagttcaacgacgagatctgcttcgccctgatcatcggcaaagagaagtataaggacaacgattactacgagatcctcaagaagcagctgttcgacctgaagattatcagccagaacatcctgtgggagaactggcggaaggacgacaagggctacatgaccaacaacctgctgatccagatcatgggcaagctgggcatcaagtatttcatcctggacagcaagaccccgtacgactacatcatgggcctcgatacaggcctgggcatcttcggcaatcacagagtcggcggctgtaccgtggtgtacgatagcgagggaaagatccggcggatccagccaatcgagacaccagctccaggcgagagactgcatctgccctacgtgatcgagtacctggaaaacaaggccaacatcgacatggaaaacaaaaacatcctgttcctccgcgacggcttcatccagaacagcgagcggaacgatctgaaagagatcagcaaagagctgaacagcaatatcgaagtgatctctattcggaagaacaacaagtacaaagtgttcaccagcgactacaggatcggcagcgtgttcggcaacgacggcatcttcctgcctcacaagacccctttcggcagcaaccctgtgaagctgagcacctggctgagattcaactgcggcaacgaggaaggcctgaaaatcaacgagagcatcatgcagctgctgtacgatctgaccaagatgaactacagcgccctgtacggcgagggcagatacctgagaatccccgctcctatccactacgccgacaagttcgtgaaggccctgggcaaaaactggaagatcgacgaggaactgctgaagcacggctttctgtacttcatc

SEQ ID NO: 5 - DNA, 70 bp, Artificial Sequence
  Description of Artificial Sequence Synthetic oligonucleotide
cctgcaggcccaggctgcctgccggggatacctcaccaagatcctgcatgccttccatggccttcttcct

SEQ ID NO: 6 - DNA, 70 bp, Artificial Sequence
  Description of Artificial Sequence Synthetic oligonucleotide
ggcaggggtctgcacccgggagcccccgttctaagctttcactgagttcatgacctacgggaacctcctg

SEQ ID NO: 7 - DNA, 24 bp, Artificial Sequence
  Description of Artificial Sequence Synthetic oligonucleotide
tgggtcccgcgggcgcccgtgcgc

SEQ ID NO: 8 - DNA, 20 bp, Artificial Sequence
  Description of Artificial Sequence Synthetic primer
cctttcccacaacgaggact

SEQ ID NO: 9 - DNA, 20 bp, Artificial Sequence
  Description of Artificial Sequence Synthetic primer
cgagaatacctccgcccctt

SEQ ID NO: 10 - DNA, 20 bp, Artificial Sequence
  Description of Artificial Sequence Synthetic primer
gctttttggtccgcatttgg

SEQ ID NO: 11 - DNA, 20 bp, Artificial Sequence
  Description of Artificial Sequence Synthetic primer
ggctctacccctagctgtct

SEQ ID NO: 12 - DNA, 89 bp, Artificial Sequence
  Description of Artificial Sequence Synthetic oligonucleotide
tctctggcttctgcaggccttgaagttgccccatgtcgactagatctaggaggactcctctgtctttgcccagagcatcccgtggaacc

SEQ ID NO: 13 - DNA, 20 bp, Artificial Sequence
  Description of Artificial Sequence Synthetic primer
ccgtgttgcagggatatggg

SEQ ID NO: 14 - DNA, 21 bp, Artificial Sequence
  Description of Artificial Sequence Synthetic primer
catttgtggggcaacaggaag

SEQ ID NO: 15 - DNA, 20 bp, Artificial Sequence
  Description of Artificial Sequence Synthetic primer
gcgtctgaattctgtggcag

SEQ ID NO: 16 - DNA, 20 bp, Artificial Sequence
  Description of Artificial Sequence Synthetic primer
ctttgccaggagcctagtgt

SEQ ID NO: 17 - DNA, 20 bp, Artificial Sequence
  Description of Artificial Sequence Synthetic primer
ttgggaccatgttggaagtt

SEQ ID NO: 18 - DNA, 20 bp, Artificial Sequence
  Description of Artificial Sequence Synthetic primer
agcactgaggttagaagctg

SEQ ID NO: 19 - DNA, 68 bp, Artificial Sequence
  Description of Artificial Sequence Synthetic oligonucleotide
acgcagtgtccgaaggtaccaaggctgtcaccaagtatacaagctccaagatggtgagcaagggcgag

SEQ ID NO: 20 - DNA, 70 bp, Artificial Sequence
  Description of Artificial Sequence Synthetic oligonucleotide
gtggctctgaaaagagcctttgagttttaaagcacctaagcacacatttacttgtacagctcgtccatgc

SEQ ID NO: 21 - DNA, 20 bp, Artificial Sequence
  Description of Artificial Sequence Synthetic primer
taacgacatcttcgagcgca

SEQ ID NO: 22 - DNA, 20 bp, Artificial Sequence
  Description of Artificial Sequence Synthetic primer
tacgacactgcattacgggg

SEQ ID NO: 23 - DNA, 21 bp, Artificial Sequence
  Description of Artificial Sequence Synthetic primer
tgtgagacttgagtggctctg

SEQ ID NO: 24 - DNA, 72 bp, Artificial Sequence
  Description of Artificial Sequence Synthetic oligonucleotide
gtgaaattgcaggatgcaggggtgtaccgcaagcttgctagcgcatgatcagctatggtggtgccgactaca

SEQ ID NO: 25 - DNA, 22 bp, Artificial Sequence
  Description of Artificial Sequence Synthetic primer
agcatttactgtcacggttccc

SEQ ID NO: 26 - DNA, 24 bp, Artificial Sequence
  Description of Artificial Sequence Synthetic primer
aaagatcaggcctctcatctataa

SEQ ID NO: 27 - DNA, 24 bp, Artificial Sequence
  Description of Artificial Sequence Synthetic oligonucleotide
tgcaggggtgtaccgctgcatgat

SEQ ID NO: 28 - DNA, 75 bp, Artificial Sequence
  Description of Artificial Sequence Synthetic oligonucleotide
gccggggatacctcaccaagatcctgcatgtcttccatggccttcttcctggcttcctggtgaagatgagtggcg

SEQ ID NO: 29 - DNA, 75 bp, Artificial Sequence
  Description of Artificial Sequence Synthetic oligonucleotide
gccggggatacctcaccaagatcctgcatgccttccatggccttcttcctggcttcctggtgaagatgagtggcg

SEQ ID NO: 30 - DNA, 74 bp, Artificial Sequence
  Description of Artificial Sequence Synthetic oligonucleotide
gccggggatacctcaccaagatccgcatgtcttccatggccttcttcctggcttcctggtgaagatgagtggcg

SEQ ID NO: 31 - DNA, 58 bp, Artificial Sequence
  Description of Artificial Sequence Synthetic oligonucleotide
gccggggatactgtcttccatggccttcttcctggcttcctggtgaagatgagtggcg

SEQ ID NO: 32 - DNA, 75 bp, Artificial Sequence
  Description of Artificial Sequence Synthetic oligonucleotide
  modified_base (26)..(26): a, c, t, g, unknown or other
gccggggatacctcaccaagatcctngcatgtcttccatggccttcttcctggcttcctggtgaagatgagtggc

SEQ ID NO: 33 - DNA, 75 bp, Artificial Sequence
  Description of Artificial Sequence Synthetic oligonucleotide
  modified_base (26)..(37): a, c, t, g, unknown or other
gccggggatacctcaccaagatcctnnnnnnnnnnnngcatgtcttccatggcctccttcctggcttcctggtga

SEQ ID NO: 34 - DNA, 59 bp, Artificial Sequence
  Description of Artificial Sequence Synthetic oligonucleotide
gccggggatacctcaccaagatcgccttcttcctggcttcctggtgaagatgagtggcg

SEQ ID NO: 35 - DNA, 66 bp, Artificial Sequence
  Description of Artificial Sequence Synthetic oligonucleotide
gccggggatacctcacccatgtcttccatggccttcttcctggcttcctggtgaagatgagtggcg

SEQ ID NO: 36 - DNA, 62 bp, Artificial Sequence
  Description of Artificial Sequence Synthetic oligonucleotide
gccggggatacctcatgtcttccatggccttcttcctggcttcctggtgaagatgagtggcg

SEQ ID NO: 37 - DNA, 46 bp, Artificial Sequence
  Description of Artificial Sequence Synthetic oligonucleotide
gccggggatacccttcttcctggcttcctggtgaagatgagtggcg

SEQ ID NO: 38 - DNA, 75 bp, Artificial Sequence
  Description of Artificial Sequence Synthetic oligonucleotide
  modified_base (26)..(29): a, c, t, g, unknown or other
gccggggatacctcaccaagatcctnnnngcatgtcttccatggccttcttcctggcttcctggtgaagatgagt

SEQ ID NO: 39 - DNA, 75 bp, Artificial Sequence
  Description of Artificial Sequence Synthetic oligonucleotide
gttgccccatgtcgactacatcgaggaggactcctctgtctttgcccagagcatcccgtggaacctggagcggat

SEQ ID NO: 40 - DNA, 75 bp, Artificial Sequence
  Description of Artificial Sequence Synthetic oligonucleotide
gttgccccatgtcgactagatctaggaggactcctctgtctttgcccagagcatcccgtggaacctggagcggat

SEQ ID NO: 41 - DNA, 75 bp, Artificial Sequence
  Description of Artificial Sequence Synthetic oligonucleotide
ccgggagcccccgttctatatcatcactgagttcatgacctacgggaacctcctggactacctgagggagtgcaa

SEQ ID NO: 42 - DNA, 75 bp, Artificial Sequence
  Description of Artificial Sequence Synthetic oligonucleotide
ccgggagcccccgttctaagctttcactgagttcatgacctacgggaacctcctggactacctgagggagtgcaa

SEQ ID NO: 43 - DNA, 66 bp, Artificial Sequence
  Description of Artificial Sequence Synthetic oligonucleotide
ccgggagcccccgttctatatgttcatgacctacgggaacctcctggactacctgagggagtgcaa

SEQ ID NO: 44 - DNA, 66 bp, Artificial Sequence
  Description of Artificial Sequence Synthetic oligonucleotide
  modified_base (32)..(32): a, c, t, g, unknown or other
gggtctgcacccgggagcccccgttctatatnttcactgagttcatgacctacgggaacctcctgg

SEQ ID NO: 45 - DNA, 297 bp, Artificial Sequence
  Description of Artificial Sequence Synthetic polynucleotide
ggatccatgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacaagctt

SEQ ID NO: 46 - DNA, 40 bp, Artificial Sequence
  Description of Artificial Sequence Synthetic oligonucleotide
cgggagcccccgttctatatcatcactgagttcatgacct